SEQUENCE LISTING (98 sequences)

SEQ ID NO: 1 - protein, 8 aa, synthetic construct
GRTFIAYA

SEQ ID NO: 2 - protein, 9 aa, synthetic construct
ITNFAGGTT

SEQ ID NO: 3 - protein, 18 aa, synthetic construct
AADRSAQTMRQVRPVLPY

SEQ ID NO: 4 - protein, 126 aa, synthetic construct [sequence omitted]

SEQ ID NO: 5 - protein, 126 aa, synthetic construct [sequence omitted]

SEQ ID NO: 6 - protein, 13 aa, Homo sapiens
FENFLHNSIFVPR

SEQ ID NO: 7 - protein, 8 aa, synthetic construct
GRTIDNYA

SEQ ID NO: 8 - protein, 8 aa, synthetic construct
  VARIANT 8: note = A or T
IDWGDGGX

SEQ ID NO: 9 - protein, 17 aa, synthetic construct
AMARQSRVNLDVARYDY

SEQ ID NO: 10 - protein, 8 aa, synthetic construct
IDWGDGGA

SEQ ID NO: 11 - protein, 124 aa, synthetic construct
QVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSS

SEQ ID NO: 12 - protein, 124 aa, synthetic construct
QVQLVESGGGLVQPGGSLRLSCAASGRTIDNYAMAWVRQAPGKGLEWVATIDWGDGGTRYANSVKGRFTISRDNSKNTMYLQMNSLRAEDTAVYYCAMARQSRVNLDVARYDYWGQGTLVTVSS

SEQ ID NO: 13 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 14 - protein, 20 aa, synthetic construct
GGGGSGGGGSGGGGSGGGGS

SEQ ID NO: 15 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 16 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 17 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 18 - protein, 552 aa, Homo sapiens
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLL

SEQ ID NO: 19 - protein, 651 aa, Homo sapiens
MARGSAVAWAALGPLLWGCALGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSLFT

SEQ ID NO: 20 - protein, 663 aa, Homo sapiens
MTGARASAAEQRRAGRSGQARAAERAAGMSGAGRALAALLLAASVLSAALLAPGGSSGRDAQAAPPRDLDKKRHAELKMDQALLLIHNELLWTNLTVYWKSECCYHCLFQVLVNVPQSPKAGKPSAAAASVSTQHGSILQLNDTLEEKEVCRLEYRFGEFGNYSLLVKNIHNGVSEIACDLAVNEDPVDSNLPVSIAFLIGLAVIIVISFLRLLLSLDDFNNWISKAISSRETDRLINSELGSPSRTDPLDGDVQPATWRLSALPPRLRSVDTFRGIALILMVFVNYGGGKYWYFKHASWNGLTVADLVFPWFVFIMGSSIFLSMTSILQRGCSKFRLLGKIAWRSFLLICIGIIIVNPNYCLGPLSWDKVRIPGVLQRLGVTYFVVAVLELLFAKPVPEHCASERSCLSLRDITSSWPQWLLILVLEGLWLGLTFLLPVPGCPTGYLGPGGIGDFGKYPNCTGGAAGYIDRLLLGDDHLYQHPSSAVLYHTEVAYDPEGILGTINSIVMAFLGVQAGKILLYYKARTKDILIRFTAWCCILGLISVALTKVSENEGFIPVNKNLWSLSYVTTLSSFAFFILLVLYPVVDVKGLWTGTPFFYPGMNSILVYVGHEVFENYFPFQWKLKDNQSHKEHLTQNIVATALWVLIAYILYRKKIFWKI

SEQ ID NO: 21 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 22 - protein, 653 aa, Homo sapiens
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 23 - protein, 743 aa, Homo sapiens
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 24 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 25 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 26 - protein, 927 aa, synthetic construct
  VARIANT 127..151: note = This region may encompass 4-5 GGGGS repeating units
  VARIANT 276..300: note = This region may encompass 4-5 GGGGS repeating units
QVQLVESGGGLVQAGGSLRLSCVASGRTFIAYAMGWFRQAPGKEREFVAAITNFAGGTTYYADSVKGRFTISRDNAKTTVYLQMNSLKPEDTALYYCAADRSAQTMRQVRPVLPYWGQGTQVTVSSGGGGSGGGGSGGGGSGGGGSGGGGSQVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSSGGGGSGGGGSGGGGSGGGGSGGGGSEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 27 - protein, 930 aa, synthetic construct
  VARIANT 127..151: note = This region may encompass 4-5 GGGGS repeating units
  VARIANT 279..303: note = This region may encompass 4-5 GGGGS repeating units
QVQLVESGGGLVQAGGSLRLSCVASGRTFIAYAMGWFRQAPGKEREFVAAITNFAGGTTYYADSVKGRFTISRDNAKTTVYLQMNSLKPEDTALYYCAADRSAQTMRQVRPVLPYWGQGTQVTVSSGGGGSGGGGSGGGGSGGGGSGGGGSQVKLEESGGGLVQAGGSLRLSCVASEYPSNFYAMSWFRQAPGKEREFVAGVSRDGLTTLYADSVKGRFTMSRDNAKNTVDLQMNSVKAEDTAVYYCAIVITGVWNKVDVNSRSYHYWGQGTQVTVSSGGGGSGGGGSGGGGSGGGGSGGGGSEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 28 - protein, 927 aa, synthetic construct
  VARIANT 127..151: note = This region may encompass 4-5 GGGGS repeating units
  VARIANT 276..300: note = This region may encompass 4-5 GGGGS repeating units
QVQLVESGGGLVQAGGSLRLSCVASGRTFIAYAMGWFRQAPGKEREFVAAITNFAGGTTYYADSVKGRFTISRDNAKTTVYLQMNSLKPEDTALYYCAADRSAQTMRQVRPVLPYWGQGTQVTVSSGGGGSGGGGSGGGGSGGGGSGGGGSQVKLEESGGGLVQAGGSLRLSCEVSGGTVSPTAMGWFRQAPGKEREFVGHITWSRGTTRVASSVKDRFTISRDSAKNTVYLQMNSLKSEDTAVYYCAASTFLRILPEESAYTYWGQGTQVTVSSGGGGSGGGGSGGGGSGGGGSGGGGSEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 29 - protein, 926 aa, synthetic construct
  VARIANT 127..151: note = This region may encompass 4-5 GGGGS repeating units
  VARIANT 275..299: note = This region may encompass 4-5 GGGGS repeating units
QVQLVESGGGLVQPGGSLRLSCAASGRTFIAYAMGWFRQAPGKEREFVAAITNFAGGTTYYADSVKGRFTISRDNAKTTVYLQMNSLRAEDTAVYYCAADRSAQTMRQVRPVLPYWGQGTLVTVSSGGGGSGGGGSGGGGSGGGGSGGGGSQVQLVESGGGLVQPGGSLRLSCAASGRTIDNYAMAWVRQAPGKGLEWVATIDWGDGGTRYANSVKGRFTISRDNSKNTMYLQMNSLRAEDTAVYYCAMARQSRVNLDVARYDWGQGTLVTVSSGGGGSGGGGSGGGGSGGGGSGGGGSEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 30 - protein, 930 aa, synthetic construct
  VARIANT 127..151: note = This region may encompass 4-5 GGGGS repeating units
  VARIANT 279..303: note = This region may encompass 4-5 GGGGS repeating units
QVQLVESGGGLVQPGGSLRLSCAASGRTFIAYAMGWFRQAPGKEREFVAAITNFAGGTTYYADSVKGRFTISRDNAKTTVYLQMNSLRAEDTAVYYCAADRSAQTMRQVRPVLPYWGQGTLVTVSSGGGGSGGGGSGGGGSGGGGSGGGGSEVQLVESGGGLVQPGGSLRLSCAASEYPSNFYAMSWFRQAPGKEREFVSGVSRDGLTTLYADSVKGRFTISRDNSKNTLYLQMNSLRAEDTAVYYCAIVITGVWNKVDVNSRSYHYWGQGTLVTVSSGGGGSGGGGSGGGGSGGGGSGGGGSEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 31 - protein, 927 aa, synthetic construct
  VARIANT 127..151: note = This region may encompass 4-5 GGGGS repeating units
  VARIANT 276..300: note = This region may encompass 4-5 GGGGS repeating units
QVQLVESGGGLVQPGGSLRLSCAASGRTFIAYAMGWFRQAPGKEREFVAAITNFAGGTTYYADSVKGRFTISRDNAKTTVYLQMNSLRAEDTAVYYCAADRSAQTMRQVRPVLPYWGQGTLVTVSSGGGGSGGGGSGGGGSGGGGSGGGGSQVQLVESGGGLVQPGGSLRLSCAVSGGTVSPTAMGWVRQAPGKGLEWVGHITWSRGTTRYASSVKGRFTISRDNSKNTVYLQMNSLRAEDTAVYYCAASTFLRILPEESAYTYWGQGTLVTVSSGGGGSGGGGSGGGGSGGGGSGGGGSEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 32 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 33 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 34 - DNA, 232 bp, synthetic construct
  source 1..232: mol_type = other DNA
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 35 - DNA, 521 bp, synthetic construct
  source 1..521: mol_type = other DNA
cctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 36 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 37 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 38 - protein, 936 aa, synthetic construct
MELGLSWVVLAALLQGVQAQVQLVESGGGLVQAGGSLRLSCVASGRTFIAYAMGWFRQAPGKEREFVAAITNFAGGTTYYADSVKGRFTISRDNAKTTVYLQMNSLKPEDTALYYCAADRSAQTMRQVRPVLPYWGQGTQVTVSSGGGGSGGGGSGGGGSGGGGSQVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSSGGGGSGGGGSGGGGSGGGGSEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 39 - DNA, 4569 bp, synthetic construct
  source 1..4569: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggagctggggctgagctgggtggtcctggctgctcttttacaaggtgtccaggctcaggtgcagctggtcgagagcgggggcgggctggtgcaggccggaggaagcctgagactgtcttgcgtggccagcggcagaacctttatcgcttacgccatgggctggtttagacaggccccaggaaaagagcgggagtttgtggcagccatcacaaatttcgccggcggtaccacctactacgccgacagcgtgaagggcagattcaccatttctagagacaacgccaagacaacagtgtacctgcagatgaattctctgaaacctgaggacacagccctgtattattgcgccgccgaccgcagtgcccagactatgaggcaggtgaggcctgtgctgccctattggggacagggcactcaggtgaccgtgagcagcggtggtggcggttcaggcggaggtggctctggcggtggcggatcgggtggcggtggctcgcaggtgaaactggaggagagcggcggcggcctggtgcaggcaggaggttccctgagactgtcttgtgccgcctcaggtagaactatcgacaactatgccatggcctggagcagacaggcccccgggaaagaccgcgagtttgtggctaccattgactggggcgacggcggcgccaggtatgcaaacagcgtgaagggcagatttaccatttctagagacaacgccaagggcactatgtatctgcagatgaataatctggagcccgaagacaccgccgtgtacagctgtgccatggccagacagtcccgggtgaatctggatgtggccaggtacgactactggggacaggggacccaggtgaccgtgtccagcggtggtggcggttcaggcggaggtggctctggcggtggcggatcgggtggcggtggctcggaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatgaacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 40 - protein, 46 aa, synthetic construct
LAEAKVLANRELDKYGVSDYYKNLINNAKTVEGVKALIDEILAALP

SEQ ID NO: 41 - protein, 9 aa, synthetic construct
GFLLRSNTM

SEQ ID NO: 42 - protein, 7 aa, synthetic construct
IRPSGLT

SEQ ID NO: 43 - protein, 10 aa, synthetic construct
HTRPPFQRDS

SEQ ID NO: 44 - protein, 10 aa, synthetic construct
ATRPPFQRDS

SEQ ID NO: 45 - protein, 9 aa, synthetic construct
GRTFDNYVM

SEQ ID NO: 46 - protein, 8 aa, synthetic construct
ISGSGSIT

SEQ ID NO: 47 - protein, 17 aa, synthetic construct
AAGSRRTYYREPKFYPS

SEQ ID NO: 48 - protein, 9 aa, synthetic construct
GSTFSSSSV

SEQ ID NO: 49 - protein, 7 aa, synthetic construct
ITSGGST

SEQ ID NO: 50 - protein, 18 aa, synthetic construct
NVAGRNWVPISRYSPGPY

SEQ ID NO: 51 - protein, 18 aa, synthetic construct
AVAGRNWVPISRYSPGPY

SEQ ID NO: 52 - protein, 116 aa, synthetic construct [sequence omitted]

SEQ ID NO: 53 - protein, 116 aa, synthetic construct
QVQLVESGGGLVQPGGSLRLSCAGPGFLLRSNTMGWYRQAPGKERELVAFIRPSGLTNYNDAVQGRFTISRDNAKNTVYLQMNSLRAEDTAVYYCHTRPPFQRDSWGQGTLVTVSS

SEQ ID NO: 54 - protein, 124 aa, synthetic construct [sequence omitted]

SEQ ID NO: 55 - protein, 124 aa, synthetic construct
EVQLVESGGGLVQPGGSLRLSCAASGRTFDNYVMAWFRQAPGKGLEFVSSISGSGSITNYANSVKDRFTISRDNSKNTLYLQMNSLRAEDTAVYYCAAGSRRTYYREPKFYPSWGQGTLVTVSS

SEQ ID NO: 56 - protein, 124 aa, synthetic construct [sequence omitted]

SEQ ID NO: 57 - protein, 124 aa, synthetic construct
EVQLVESGGGLVQPGGSLRLSCAASGSTFSSSSVGWYRQAPGKGLELVSAITSGGSTNTADSVKGRFTISRDNSKNTLYLQMNSLRAEDTAVYYCNVAGRNWVPISRYSPGPYWGQGTLVTVSS

SEQ ID NO: 58 - protein, 8 aa, synthetic construct
EYPSNFYA

SEQ ID NO: 59 - protein, 8 aa, synthetic construct
VSRDGLTT

SEQ ID NO: 60 - protein, 20 aa, synthetic construct
AIVITGVWNKVDVNSRSYHY

SEQ ID NO: 61 - protein, 8 aa, synthetic construct
GGTVSPTA

SEQ ID NO: 62 - protein, 8 aa, synthetic construct
ITWSRGTT

SEQ ID NO: 63 - protein, 17 aa, synthetic construct
AASTFLRILPEESAYTY

SEQ ID NO: 64 - protein, 124 aa, synthetic construct
  VARIANT 1: note = E or Q
  VARIANT 3: note = K or Q
  VARIANT 5: note = V or E
  VARIANT 14: note = A or P
  VARIANT 37: note = V or S
  VARIANT 44: note = D or G
  VARIANT 45: note = L or R
  VARIANT 47: note = F or W
  VARIANT 49: note = A or S
  VARIANT 58: note = A or T
  VARIANT 75: note = A or S
  VARIANT 77: note = G or N
  VARIANT 79: note = M or L
  VARIANT 85: note = N or R
  VARIANT 87: note = E or R
  VARIANT 88: note = P or A
  VARIANT 95: note = S or Y
  VARIANT 119: note = Q or L
XVXLXESGGGLVQXGGSLRLSCAASGRTIDNYAMAWXRQAPGKXXEXVXTIDWGDGGXRYANSVKGRFTISRDNXKXTXYLQMNXLXXEDTAVYXCAMARQSRVNLDVARYDYWGQGTXVTVSS

SEQ ID NO: 65 - protein, 128 aa, synthetic construct
  VARIANT 1: note = E or Q
  VARIANT 3: note = K or Q
  VARIANT 5: note = V or E
  VARIANT 14: note = A or P
  VARIANT 23: note = V or A
  VARIANT 37: note = F or V
  VARIANT 44: note = E or G
  VARIANT 45: note = R or L
  VARIANT 47: note = F or W
  VARIANT 49: note = A or S
  VARIANT 70: note = M or I
  VARIANT 75: note = A or S
  VARIANT 80: note = V or L
  VARIANT 81: note = D or Y
  VARIANT 87: note = V or L
  VARIANT 88: note = K or R
  VARIANT 123: note = Q or L
XVXLXESGGGLVQXGGSLRLSCXASEYPSNFYAMSWXRQAPGKXXEXVXGVSRDGLTTLYADSVKGRFTXSRDNXIKNTXXLQMNSXXAEDTAVYYCAIVITGVWNKVDVNSRSYHYWGQGTXVTVSS

SEQ ID NO: 66 - protein, 124 aa, synthetic construct
  VARIANT 1: note = E or Q
  VARIANT 3: note = K or Q
  VARIANT 5: note = V or E
  VARIANT 14: note = A or P
  VARIANT 23: note = A or E
  VARIANT 24: note = V or A
  VARIANT 37: note = V or F
  VARIANT 44: note = G or E
  VARIANT 45: note = L or R
  VARIANT 47: note = F or W
  VARIANT 49: note = G or S
  VARIANT 61: note = V or Y
  VARIANT 67: note = D or G
  VARIANT 75: note = N or S
  VARIANT 76: note = A or S
  VARIANT 80: note = L or V
  VARIANT 88: note = K or R
  VARIANT 89: note = A or S
  VARIANT 120: note = L or Q
XVXLXESGGGLVQXGGSLRLSCXXSGGTVSPTAMGWXRQAPGKXXEXVXHITWSRGTTRXASSVKXRFTISRDXXKNTXYLQMNSLXXEDTAVYYCAASTFLRILPEESAYTYWGQGTXVTVSS

SEQ ID NO: 67 - protein, 127 aa, synthetic construct
QVKLEESGGGLVQAGGSLRLSCVASEYPSNFYAMSWFRQAPGKEREFVAGVSRDGLTTLYADSVKGRFTMSRDNAKNTVDLQMNSVKAEDTAVYYCAIVITGVWNKVDVNSRSYHYWGQGTQVTVSS

SEQ ID NO: 68 - protein, 127 aa, synthetic construct
EVQLVESGGGLVQPGGSLRLSCAASEYPSNFYAMSWFRQAPGKEREFVSGVSRDGLTTLYADSVKGRFTISRDNSKNTLYLQMNSLRAEDTAVYYCAIVITGVWNKVDVNSRSYHYWGQGTLVTVSS

SEQ ID NO: 69 - protein, 124 aa, synthetic construct
QVKLEESGGGLVQAGGSLRLSCEVSGGTVSPTAMGWFRQAPGKEREFVGHITWSRGTTRVASSVKDRFTISRDSAKNTVYLQMNSLKSEDTAVYYCAASTFLRILPEESAYTYWGQGTQVTVSS

SEQ ID NO: 70 - protein, 124 aa, synthetic construct
QVQLVESGGGLVQPGGSLRLSCAVSGGTVSPTAMGWVRQAPGKGLEWVGHITWSRGTTRYASSVKGRFTISRDNSKNTVYLQMNSLRAEDTAVYYCAASTFLRILPEESAYTYWGQGTLVTVSS

SEQ ID NO: 71 - protein, 4 aa, synthetic construct
GGGG

SEQ ID NO: 72 - protein, 5 aa, synthetic construct
TGGGG

SEQ ID NO: 73 - protein, 20 aa, synthetic construct
GGSSGGSGSSSGSGGSGSSG

SEQ ID NO: 74 - protein, 40 aa, synthetic construct
  VARIANT 1..40: note = This sequence may encompass 1-10 GGSS repeating units
GGSSGGSSGGSSGGSSGGSSGGSSGGSSGGSSGGSSGGSS

SEQ ID NO: 75 - protein, 50 aa, synthetic construct
  VARIANT 1..50: note = This sequence may encompass 1-10 SGGGG repeating units
SGGGGSGGGGSGGGGSGGGGSGGGGSGGGGSGGGGSGGGGSGGGGSGGGG

SEQ ID NO: 76 - protein, 54 aa, synthetic construct
  VARIANT 5..54: note = This region may encompass 1-10 SGGGG repeating units
GGGGSGGGGSGGGGSGGGGSGGGGSGGGGSGGGGSGGGGSGGGGSGGGGSGGGG

SEQ ID NO: 77 - protein, 22 aa, synthetic construct
SKPTCPPPELLGGPSVFIFPPK

SEQ ID NO: 78 - protein, 25 aa, synthetic construct
GGGGSGGGGSGGGGSGGGGSGGGGS

SEQ ID NO: 79 - protein, 19 aa, synthetic construct
MELGLSWVVLAALLQGVQA

SEQ ID NO: 80 - protein, 18 aa, Homo sapiens
MKWVTFISLLFLFSSAYS

SEQ ID NO: 81 - protein, 20 aa, Homo sapiens
MVLQTQVFISLLLWISGAYG

SEQ ID NO: 82 - protein, 23 aa, Homo sapiens
MQMSPALTCLVLGLALVFGEGSA

SEQ ID NO: 83 - protein, 18 aa, Homo sapiens
MGLAWGLGVLFLMHVCGT

SEQ ID NO: 84 - protein, 31 aa, Homo sapiens
MLRGPGPGLLLLAVQCLGTAVPSTGASKSKR

SEQ ID NO: 85 - protein, 21 aa, Homo sapiens
MGWRAAGALLLALLLHGRLLA

SEQ ID NO: 86 - protein, 29 aa, Homo sapiens
MAGRSHPGPLRPLLPLLVVAACVLPGAGG

SEQ ID NO: 87 - skipped

SEQ ID NO: 88 - protein, 4 aa, synthetic construct
GRGD

SEQ ID NO: 89 - protein, 6 aa, synthetic construct
GRGDSP

SEQ ID NO: 90 - protein, 7 aa, synthetic construct
GGRGDSP

SEQ ID NO: 91 - protein, 8 aa, synthetic construct
GGGRGDSP

SEQ ID NO: 92 - protein, 5 aa, synthetic construct
GGGGS

SEQ ID NO: 93 - protein, 849 aa, synthetic construct
  VARIANT 127..151: note = This region may encompass 4-5 GGGGS repeating units
  VARIANT 275..299: note = This region may encompass 4-5 GGGGS repeating units
QVQLVESGGGLVQPGGSLRLSCAASGRTFIAYAMGWFRQAPGKEREFVAAITNFAGGTTYYADSVKGRFTISRDNAKTTVYLQMNSLRAEDTAVYYCAADRSAQTMRQVRPVLPYWGQGTLVTVSSGGGGSGGGGSGGGGSGGGGSGGGGSQVQLVESGGGLVQPGGSLRLSCAASGRTIDNYAMAWVRQAPGKGLEWVATIDWGDGGTRYANSVKGRFTISRDNSKNTMYLQMNSLRAEDTAVYYCAMARQSRVNLDVARYDWGQGTLVTVSSGGGGSGGGGSGGGGSGGGGSGGGGSMPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 94 - protein, 849 aa, synthetic construct
  VARIANT 124..148: note = This region may encompass 4-5 GGGGS repeating units
  VARIANT 275..299: note = This region may encompass 4-5 GGGGS repeating units
QVQLVESGGGLVQPGGSLRLSCAASGRTIDNYAMAWVRQAPGKGLEWVATIDWGDGGTRYANSVKGRFTISRDNSKNTMYLQMNSLRAEDTAVYYCAMARQSRVNLDVARYDWGQGTLVTVSSGGGGSGGGGSGGGGSGGGGSGGGGSQVQLVESGGGLVQPGGSLRLSCAASGRTFIAYAMGWFRQAPGKEREFVAAITNFAGGTTYYADSVKGRFTISRDNAKTTVYLQMNSLRAEDTAVYYCAADRSAQTMRQVRPVLPYWGQGTLVTVSSGGGGSGGGGSGGGGSGGGGSGGGGSMPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 95 - protein, 609 aa, Homo sapiens [sequence omitted]

SEQ ID NO: 96 - protein, 25 aa, synthetic construct
  VARIANT 1..25: note = This sequence may encompass 4-5 GGGGS repeating units
GGGGSGGGGSGGGGSGGGGSGGGGS

SEQ ID NO: 97 - protein, 25 aa, synthetic construct
  VARIANT 1..25: note = This sequence may encompass 3-5 GGGGS repeating units
GGGGSGGGGSGGGGSGGGGSGGGGS

SEQ ID NO: 98 - protein, 15 aa, synthetic construct
GGGGSGGGGSGGGGS

(19) United States
(12) Patent Application Publication
(10) Pub. No.: US 2025/0144174 A1
(43) Pub. Date: May 8, 2025

(54) COMPOSITIONS, DEVICES, AND METHODS FOR TREATING CNS DISORDERS

(71) Applicants: SIGILON THERAPEUTICS, INC, Cambridge, MA (US); National Research Council of Canada, Ottawa (CA)

(72) Inventors: Elina Makino, Winchester, MA (US); Erika Pearson, Mansfield, MA (US); Gregory Hussack, Ottawa (CA); Danica Stanimirovic, Ottawa (CA)

(73) Assignee: National Research Council of Canada, Ottawa, ON (CA)

(21) Appl. No.: 18/833,641

(22) PCT Filed: Jan. 27, 2023
(86) PCT No.: PCT/US2023/011727

§ 371 Date: Jul. 26, 2024

Related U.S. Application Data
(60) Provisional application No. 63/304,377, filed on Jan. 28, 2022.

(51) Int. Cl. A61K 38/17; A61K 9/48; A61K 35/12; A61K 47/36; C07K 14/71; C12N 5/0735; C12N 5/0775

(52) U.S. Cl. CPC A61K 38/179; A61K 9/4891; A61K 35/12; A61K 47/36; C07K 14/71; C12N 5/0606; C12N 5/0663; C07K 2317/51; C07K 2317/56; C07K 2317/92; C12N 2510/00

(57) ABSTRACT

Described herein are BBB-transporting fusion proteins, polynucleotides encoding the fusion proteins and mammalian cells genetically modified to express and secrete the fusion proteins, as well as compositions, implantable devices and device preparations comprising the fusion proteins or genetically modified cells secreting the fusion proteins, and methods of making and using the same for treating various CNS diseases and conditions, e.g., any mucopolysaccharidosis type with CNS manifestations.

### CLAIM OF PRIORITY

The present application claims priority to U.S. Patent Application No. 63/304,377, filed Jan. 28, 2022. The entire contents of the foregoing application is incorporated herein by reference in its entirety.

### BACKGROUND

A number of neurodegenerative and other central nervous system (CNS) disorders are theoretically treatable by proteins such as enzymes, cytokines and antibodies. However, the promise of using protein-based therapies to treat CNS disorders has been limited by the difficulty in transporting molecules larger than 500 daltons across the blood-brain barrier (BBB), which is formed by brain endothelial cells (BECs) that line the blood vessels and connect with each other through tight junctions. Receptor-mediated transcytosis (RMT) is one of a variety of approaches that has been explored to increase delivery of proteins across the BBB. The RMT approach typically employs intravenous or subcutaneous administration of a fusion protein that links the therapeutic protein to a molecule that binds one of the endogenous receptors expressed on the surface of the BECs. Following receptor binding, the RMT process requires internalization of the receptor-fusion protein complex and exocytosis of the fusion protein on the abluminal side of the cell. Candidate target receptors for RMT include the transferrin receptor, insulin receptor, insulin-like growth factor-1 receptor (IGF1R), low-density lipoprotein (LDL) receptor related proteins 1 and 2 (LRP-1 and LRP-2), diptheria toxin receptor and TMEM30A. Results obtained in pre-clinical and clinical studies indicate that a variety of factors can impact the amount and/or activity of therapeutic protein that crosses the BBB, including plasma half-life of the fusion protein and its binding affinity for the RMT target receptor.

### SUMMARY

Described herein are BBB-transporting fusion proteins that comprise a cargo moiety, e.g., a molecule with an activity useful for treating or diagnosing a central nervous system (CNS) condition or disorder of interest, located C-terminal to separate domains that bind to human serum albumin (HSA) and to the extracellular portion of human IGF1R (hIGF1R). In an embodiment, the IGF1R binding domain is disposed between the HSA-binding domain and the cargo moiety. In another embodiment, the HSA-binding domain is disposed between the IGF1R binding domain and the cargo moiety. In an embodiment, a linker moiety is disposed in between the two binding domains and/or between the hIGF1-binding domain and the cargo moiety. In an embodiment, one or both of the HSA-binding and hIGF1R-binding domains has a molecular weight of less than about any of 75 kDa, 50 kDa or 25 kDa. In an embodiment, the amino acid sequence of each of the HSA-binding and hIGF1R-binding domains is from a single chain Fab (scFab), a single chain Fv (scFv) or a single domain antibody (sdAb). In an embodiment, the cargo moiety (molecule) has a molecular weight of about 1 kD to about 200 kD, or about 2 kD to about 100 kD. In an embodiment, the cargo moiety (molecule) consists essentially of, or consists of the amino acid sequence of a polypeptide, e.g., a cytokine, an enzyme, or an antibody. In an embodiment, the linker moiety is a linker peptide that is less than 50 amino acids in length.

In one aspect, the present disclosure features a BBB-transporting fusion protein which comprises a primary structure defined by formula I: AB-L1-RB-L2-C or by formula II: RB-L1-AB-L2-C, wherein in each formula AB comprises an HSA-binding domain, L1 comprises a first linker amino acid sequence, RB comprises a hIGF1R-binding domain, L2 comprises a second linker amino acid sequence, and C comprises the amino acid sequence of a cargo polypeptide.

In an embodiment, the fusion protein binds via AB to domain 1 (DI) or domain 2 (DII) of HSA and does not substantially inhibit binding of human FcRn (h-FcRn) to HSA. In an embodiment, the fusion protein binds via the AB domain to HSA with a K_{D} affinity of less than about 1 nM to about 100 nM within a pH range of about 5.0 to about 7.4 as determined by surface plasmon resonance at 25° C. In some embodiments, the fusion protein also binds via AB to at least one mammalian serum albumin ortholog at 25° C. within a pH range of about 5.5 to about 7.4. In an embodiment, the albumin ortholog is from mouse, rat, hamster, rabbit, guinea pig, pig, cat, dog, or a non-human primate (e.g., cynomolgus or rhesus monkey).

In an embodiment, AB comprises first, second and third amino acid sequences corresponding to the three complementarity determining regions CDR1, CDR2 and CDR3 of the heavy chain variable region of an anti-HSA antibody (e.g., a conventional antibody with two heavy chains and two light chains, an scFab, an scFv, a sdAb). In an embodiment, the CDR1, CDR2 and CDR3 amino acid sequences in AB are: GRTFIAYA (SEQ ID NO:1) or a conservatively substituted variant thereof; ITNFAGGTT (SEQ ID NO:2) or a conservatively substituted variant thereof; and AADRSAQTMRQVRPVLPY (SEQ ID NO:3) or a conservatively substituted variant thereof.

In an embodiment, AB consists essentially of, or consists of: QVQLVESGGGLVQAGGSLRLSCVASGRTFIAYAMGWFRQAPGKEREFVAAITNFAGGT TYYADSVKGRFTISRDNAKTTVYLQMNSLKPEDTALYYCAADRSAQTMRQVRPVLPY WGQGTQVTVSS (SEQ ID NO:4), or a conservatively substituted variant thereof. In an embodiment, AB consists essentially of, or consists of: QVQLVESGGGLVQPGGSLRLSCAASGRTFIAYAMGWFRQAPGKEREFVAAITNFAGGT TYYADSVKGRFTISRDNAKTTVYLQMNSLRAEDTAVYYCAADRSAQTMRQVRPVLPY WGQGTLVTVSS (SEQ ID NO:5), or a conservatively substituted variant thereof. In an embodiment, AB consists essentially of, or consists of, an amino acid sequence of the heavy chain variable region of an antibody that cross-competes with a sdAb consisting of SEQ ID NO:4 or SEQ ID NO:5 for binding to HSA.

The fusion protein binds via the RB domain to hIGF1R expressed on the surface of human brain endothelial cells. In some embodiments, the fusion protein does not substantially bind to the human insulin receptor (h-IR). In some embodiments, the fusion protein does not substantially inhibit binding of insulin, insulin growth factor 1 (IGF1) or insulin growth factor 2 (IGF2) to hIGF1R. In an embodiment, the fusion protein binds via RB to an epitope in the hIGF1R extracellular domain which comprises FENFLHNSIFVPR (SEQ ID NO:6). In an embodiment, the fusion protein binds via RB to hIGF1R with a K_{D} affinity of about 0.5 nM to about 50 nM within a pH range of about 5.0 to about 7.4, as determined by surface plasmon resonance at 25° C. In some embodiments, the fusion protein also binds via RB to at least one mammalian IGF1R ortholog at 25° C. and within a pH range of about 5.0 to about 7.4. In an embodiment, the IGF1R ortholog is from mouse, rat, hamster, rabbit, guinea pig, dog, cat, or a non-human primate (e.g., cynomolgus or rhesus monkey). In an embodiment, RB comprises first, second and third amino acid sequences corresponding to the three complementarity determining regions CDR1, CDR2 and CDR3 of the heavy chain variable region of an anti-hIGF1R antibody (e.g., a conventional antibody with two heavy chains and two light chains, an scFab, an scFv, a sdAb). In an embodiment, the CDR1, CDR2 and CDR3 amino acid sequences in RB are GRTIDNYA (SEQ ID NO:7) or a conservatively substituted variant thereof; IDWGDGGX, where X is A or T (SEQ ID NO:8) or a conservatively substituted variant thereof; and AMARQSRVNLDVARYDY (SEQ ID NO:9) or a conservatively substituted variant thereof. In an embodiment, the CDR2 sequence in RB is IDWGDGGA (SEQ ID NO:10).

In an embodiment, RB consists essentially of, or consists of: QVKLEESGGGLVQAGGSLRLSCAASGRTIDNYAMAWSRQAPGKDREFVATIDWGDGG ARYANSVKGRFTISRDNAKGTMYLQMNNLEPEDTAVYSCAMARQSRVNLDVARYDY WGQGTQVTVSS (SEQ ID NO:11) or a conservatively substituted variant thereof. In another embodiment, RB consists essentially of, or consists of: QVQLVESGGGLVQPGGSLRLSCAASGRTIDNYAMAWVRQAPGKGLEWVATIDWGDGG TRYANSVKGRFTISRDNSKNTMYLQMNSLRAEDTAVYYCAMARQSRVNLDVARYDY WGQGTLVTVSS (SEQ ID NO:12) or a conservatively substituted variant thereof. In an embodiment, RB consists essentially of, or consists of, an amino acid sequence of the heavy chain variable region of an antibody that cross-competes with a sdAb consisting of SEQ ID NO:11 or SEQ ID NO:12 for binding to hIGF1R.

In some embodiments, the cargo moiety in the fusion protein comprises, consists essentially of, or consists of the amino acid sequence for an enzyme that is deficient in a lysosomal storage disorder (LSD), e.g., alpha-L-iduronidase (IDUA), iduronate-2-sulfatase (IDS), arylsulfatase B (ARSB), N-sulfoglucosamine sulfohydrolase (SGSH), glucosylceramidase (GBA), alpha-galactosidase A (GLA), and alpha-1,4-glucosidase (GAA). In an embodiment, the cargo moiety is not IDS. In other embodiments, the cargo moiety (molecule) comprises, consists essentially of, or consists of the amino acid sequence for an antibody or antigen-binding fragment thereof that binds to a target protein in the brain, e.g., beta-secretase 1 (BACE1), an immunotherapy target (e.g., programmed death receptor 1 (PD-1)).

The L1 and L2 amino acid sequences in a BBB-transporting fusion protein of the disclosure may be the same or different. In some embodiments, each of L1 and L2 is 3 to about 35 amino acids in length, 4 to about 30 amino acids in length or 5 to about 20 amino acids in length. In an embodiment, each of L1 and L2 consists essentially of, or consists of, (GGGGS)ₙ (SEQ ID NO:13), where n is equal to 3, 4 or 5. In an embodiment, each of L1 and L2 consists essentially of, or consists of, (GGGGS)₄ (SEQ ID NO:14). In an embodiment, the cargo moiety in the BBB-transporting fusion protein is an IDUA protein. In an embodiment, the BBB-transporting IDUA fusion protein comprises, consists essentially of, or consists of the amino acid sequence shown in FIG. 6 (SEQ ID NO:38).

In another aspect, the present disclosure provides a polynucleotide (e.g., an isolated polynucleotide) which comprises a nucleotide sequence that encodes a BBB-transporting fusion protein described herein. In an embodiment, the nucleotide sequence is operably linked to a promoter sequence and a polyA signal sequence. In an embodiment, the promoter sequence is identical to, or substantially identical to, one of the promoter sequences in FIG. 4 (SEQ ID NO:32, SEQ ID NO:33 or SEQ ID NO:34). In an embodiment, the polyA signal sequence is identical to, or substantially identical to, one of the polyA signal sequences shown in FIG. 5 (SEQ ID NO:35, SEQ ID NO:36 or SEQ ID NO:37). In an embodiment, the BBB-transporting fusion protein consists of the amino acid sequence shown in FIG. 6 and the polynucleotide comprises, consists essentially or, or consists of the nucleotide sequence shown in FIG. 7.

In yet another aspect, the present disclosure provides a mammalian cell (e.g., a mouse cell, a Chinese hamster ovary (CHO) cell, a monkey cell, a human cell (e.g., an RPE cell) that is genetically modified to express and secrete a BBB-transporting fusion protein described herein. In an embodiment, the mammalian cell is genetically modified by transfection with a polynucleotide described herein (e.g., an expression vector) which encodes the BBB-transporting fusion protein. In an embodiment, the cargo moiety in the fusion protein is an IDUA protein. In an embodiment, the mammalian cell is derived from an ARPE-19 cell by transfection with an expression vector comprising the nucleotide sequence shown in FIG. 7.

The present disclosure also provides a composition comprising a plurality of genetically modified cells described herein and a method of manufacturing the composition. In an embodiment, the composition comprises a cell culture media or a storage medium. In an embodiment, the composition comprises a polymer solution in which the cells are suspended, e.g., a polymer solution described herein, e.g., comprising alginate and a cell-binding substance, e.g., as defined herein. In an embodiment, the method of manufacturing the composition comprises culturing a plurality of a genetically modified cell described herein until a desired number of cultured cells has been produced, and combining the desired number of cultured cells with a cell culture media, a storage medium or a polymer solution.

In yet another aspect, the present disclosure features a device comprising at least one cell-containing compartment which comprises a genetically modified mammalian cell as described herein or a plurality of such cells. The device is configured to shield the cell(s) from the recipient's immune system and mitigate the foreign body response (FBR) (as defined herein) to the implanted device. In an embodiment, the surface of the device comprises a compound or polymer that mitigates the FBR (as defined herein) to the device (e.g., an afibrotic compound or afibrotic polymer). In an embodiment, an afibrotic polymer comprises a biocompatible, zwitterionic polymer, e.g., as described in WO 2017/218507, WO 2018/140834, or Liu et al., Zwitterionically modified alginates mitigate cellular overgrowth for cell encapsulation, Nature Communications (2019)10:5262. In an embodiment, the compound is a compound of Formula (III):

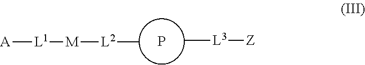

or a pharmaceutically acceptable salt thereof, wherein the variables A, L¹, M, L², P, L³, and Z, as well as related subvariables, are defined herein. In some embodiments, the compound of Formula (III) or a pharmaceutically acceptable salt thereof (e.g., Formulas (III-a), (III-b), (III-b-i), (III-b-ii), (III-c), (III-d), (III-e), (III-f), (IV), (IV-a), (V), (V-a), (V-b), (V-c), (V-d), (VI-a), (VI-b), (VI-c), (VI-d), or (VI-e)) is a compound described herein, including for example, one of the compounds shown in Table 6 herein. In an embodiment, the compound of Formula (III) is a compound selected from Compound 100, Compound 101, Compound 102 or Compound 122 shown in Table 6.

In one aspect, a device of the disclosure is a two-compartment hydrogel capsule (e.g., a microcapsule (less than 1 mm in diameter) or a millicapsule (at least 1 mm in diameter)) in which a cell-containing compartment (e.g., the inner compartment) comprising a plurality of live genetically modified cells described herein (and optionally one or more cell binding substances) is surrounded by a barrier compartment comprising an afibrotic polymer (e.g., the outer compartment, e.g., hydrogel layer). In an embodiment, the afibrotic polymer comprises an afibrotic compound. In an embodiment, the afibrotic compound is a compound of Formula (III).

In another aspect, the present disclosure features a preparation (e.g., a composition) comprising a plurality (at least any of 3, 6, 12, 25, 50 or more) of a cell-containing device described herein, e.g, a preparation of hydrogel capsules encapsulating genetically modified ARPE-19 cells. In some embodiments, the preparation is a pharmaceutically acceptable composition.

In another aspect, the present disclosure features a method of making or manufacturing a device comprising a genetically modified cell described herein. In some embodiments, the method comprises providing the genetically modified cell, or a plurality of such cells, and disposing the cell(s) in an enclosing component, e.g., a cell-containing compartment of the device as described herein. In some embodiments, the enclosing component comprises a flexible polymer (e.g., PLA, PLG, PEG, CMC, or a polysaccharide, e.g., alginate). In some embodiments, the enclosing component comprises an inflexible polymer or metal housing. In some embodiments, the surface of the device is chemically modified, e.g., with a compound of Formula (III) as described herein.

In an embodiment, a device described herein, or a plurality of the device, is combined with a pharmaceutically acceptable excipient to prepare a device preparation or a composition which may be administered to a subject (e.g., into the intraperitoneal cavity) in need of treatment with the BBB-transporting fusion protein produced by the device. In an embodiment, the genetically modified cells are derived from a human cell (e.g., an RPE cell, an ARPE-19 cell) and the device preparation or composition is capable of continuously delivering an effective amount of the BBB-transporting fusion protein to the subject for a sustained time period, e.g., at least any of 3 months, 6 months, one year, two years or longer.

In another aspect, the present disclosure features a method of evaluating a composition, device or device preparation described herein. In some embodiments, the method comprises providing the composition, device or device preparation and evaluating a functional parameter of the composition, device or device preparation. In an embodiment, the functional parameter is the amount of the BBB-transporting fusion protein produced by the cells in the composition, device or device preparation in vitro (e.g., when placed in a suitable culture medium) and/or in vivo (e.g., after implant into a subject, e.g., a non-human subject or a human subject.

In another aspect, the present disclosure features a method of treating a subject in need of therapy with a BBB-transporting fusion protein described herein. In an embodiment, the method comprises administering to the subject an implantable element (e.g., a device or device preparation) comprising a genetically modified cell that expresses and secrets the fusion protein, or a plurality of such cells. In some embodiments, the administering step comprises placing into the subject a pharmaceutically acceptable preparation comprising a plurality of devices, each of which has the ability to produce the BBB-transporting fusion protein. In some embodiments, the implantable element is administered to, placed in, or injected in the peritoneal cavity (e.g., the lesser sac), the omentum, or the subcutaneous fat of a subject. In an embodiment, the method further comprises measuring the amount of the BBB-transporting fusion protein present in a tissue sample removed from the subject, e.g., in plasma separated from a blood sample or in a tissue sample obtained from the CNS or from an organ of interest. In an embodiment, the tissue sample is removed from the patient at 15, 30, 60 or 120 days after administration, implantation, or placement of the device or device preparation. In some embodiments, the subject is a human. In an embodiment, the subject is a human patient diagnosed as having a neuronopathic MPS disease and the fusion protein comprises the glycosaminoglycan-metabolizing enzyme that is deficient in the neuronopathic MPS disease. In an embodiment, the implantable element produces the BBB-transporting fusion protein in an amount sufficient to reduce one or more symptoms of the neuronopathic MPS disease. In an embodiment, the treatment results in a reduction in heparan sulfate levels in the brain and optionally in one or more other organs or tissues outside the CNS, e.g., liver, spleen, kidney, lung and heart. In an embodiment, the patient has been diagnosed with Mucopolysaccharidosis type I (MPS1) and the BBB-transporting fusion protein comprises a human IDUA protein.

The details of one or more embodiments of the disclosure are set forth herein. Other features, objects, and advantages of the disclosure will be apparent from the Detailed Description, the Figures, the Examples, and the Claims.

### BRIEF DESCRIPTION OF THE DRAWINGS

FIGS. 1A-1J show the amino acid sequences of the wild-type, human precursor polypeptides for exemplary LSD enzymes that may be included as the cargo polypeptide in BBB-transporting fusion proteins described herein: GAA (FIG. 1A, SEQ ID NO:15); GBA (FIG. 1B, SEQ ID NO:16); GLA; (FIG. 1C, SEQ ID NO:17); GNS (FIG. 1D, SEQ ID NO:18); GUSB (FIG. 1E, SEQ ID NO:19); HGSNAT (FIG. 1F, SEQ ID NO:20); IDS (FIG. 1G, SEQ ID NO:21); IDUA (FIG. 1H, SEQ ID NO:22); NAGLU (FIG. 1I, SEQ ID NO:23); and SGSH (FIG. 1J, SEQ ID NO:24).

FIGS. 2A-2B show exemplary amino acid sequences of the precursor forms of human proteins bound by BBB-transporting fusion proteins described herein, with FIG. 2A (SEQ ID NO:25) showing the amino acid sequence of a precursor human IGF1R monomer (UniProtKB—P08069), with the signal peptide italicized, the furin cleavage site shown in lower case italics, and underlined bold font indicating amino acids that are points of contact in putative binding sites for certain hIGF1R-binding domains described herein and FIG. 2B (SEQ ID NO:95) showing the amino acid sequence of precursor human serum albumin, with the signal peptide underlined.

FIGS. 3A-3F show the amino acid sequences of exemplary BBB-transporting IDUA fusion proteins of the disclosure, with the fusion proteins in FIG. 3A (SEQ ID NO:26), FIG. 3B (SEQ ID NO:27) and FIG. 3C (SEQ ID NO:28) comprising a parental anti-HSA and one of three different parental anti-IGF1R sdAb sequences; the fusion proteins in FIG. 3D (SEQ ID NO:29), FIG. 3E (SEQ ID NO:30), and FIG. 3F (SEQ ID NO:31) comprising humanized variants of the anti-HSA and anti-IGF1R sdAb sequences shown in FIGS. 3A, 3B and 3C; and each of the fusion proteins comprising two linker sequences with 4 or 5 repeats of GGGGS (SEQ ID NO:92.

FIGS. 4A-4C show exemplary promoter sequences that are useful in an expression construct for BBB-transporting fusion proteins of the disclosure: pCAG promoter sequence (FIG. 4A, SEQ ID NO:32); EF1α promoter sequence (FIG. 4B, SEQ ID NO:33); and EFS promoter sequence (FIG. 4C, SEQ ID NO:34).

FIGS. 5A-5C show exemplary polyA signal sequences that are useful in an expression construct for BBB-transporting fusion proteins of the disclosure: rBG poly A signal sequence (FIG. 5A, SEQ ID NO:35); SV40 late poly A signal sequence (FIG. 5B, SEQ ID NO:36) and BGH poly A signal sequence (FIG. 5C, SEQ ID NO:37).

FIG. 6 shows the amino acid sequence (SEQ ID NO:38) for an exemplary BBB-transporting IDUA fusion protein, with underlining indicating a VHH consensus signal peptide, bold font indicating the anti-HSA sdAb, italics indicating the anti-IGF1R sdAb, bold italics font indicating the wild-type, human mature IDUA amino acid sequence and dash-dot underlining indicating the flexible linker sequences.

FIG. 7 shows the nucleotide sequence (SEQ ID NO:39) of an exemplary transcription unit useful for expressing the IDUA fusion protein described in FIG. 6, with wavy underlining indicating the EF1A promoter sequence, straight underlining indicating an exemplary coding sequence for the VHH consensus signal peptide, bold font indicating an exemplary coding sequence for the anti-HSA sdAb, italics indicating an exemplary coding sequence for the anti-IGF1R sdAb, bold italics indicating an exemplary coding sequence for the wild-type, human mature IDUA amino acid sequence, dash-dot underline indicating a coding sequence for the flexible inker sequences, dotted underline indicating the stop codon and underlined italics font indicating the rBG poly A signal sequence.

FIGS. 8A-8B illustrate IDUA activity in single IDUA fusion proteins containing an exemplary anti-IGF1R sdAb fused to hIDUA via an amino acid linker, with FIG. 8A showing in vitro IDUA activity in culture media of cells expressing one of six different fusion constructs based on the sdAb orientation and linker length, and FIG. 8B comparing in vivo hIDUA activity in liver and plasma samples from MPS-1 mice implanted with encapsulated cells expressing either wild-type hIDUA (light grey bars) or the fusion construct that had produced the highest in vitro hIDUA activity in FIG. 8A (IGF1rR-hIDUA, dark grey bars).

FIG. 9A is a graph showing the in vitro IDUA activity in conditioned culture media of cells expressing one of six different double IDUA fusion proteins containing various orientations of hIDUA, an exemplary anti-IGF1R sdAb (IGF1R5) and an exemplary anti-HSA sdAb (R28).

FIG. 9B is a graph comparing the in vitro IDUA activity in conditioned culture media of cells expressing an exemplary anti-IGF1R-hIDUA fusion enzyme (IGF1R4-hIDUA, light grey bar) with cells expressing an exemplary anti-HSA-anti-IGF1R-hIDUA fusion enzyme (R28-IGF1R5-hIDUA, dark grey bar).

FIG. 10 is a graph comparing in vivo hIDUA activity in plasma and systemic (non-brain) tissues of MPS-1 mice implanted with encapsulated cells expressing either wild-type hIDUA (light grey bars) or an exemplary anti-HSA-anti-IGF1R-hIDUA fusion enzyme (R28-IGF1R5-hIDUA, dark grey bars).

FIG. 11 is a graph comparing heparan sulfate levels in brain tissue samples from untreated MPS-1 mice (light grey bars) and MPS-1 mice implanted with encapsulated cells expressing an exemplary anti-HSA-anti-IGF1R-hIDUA fusion enzyme (R28-IGF1R5-hIDUA, dark grey bars).

FIGS. 12A and 12B are amino acid sequences (SEQ ID NO:93 and SEQ ID NO:94) of exemplary BBB-transporting IDS fusion proteins of the disclosure.

FIG. 13 is a chart illustrating a comparison of enzyme activity levels generated with exemplary constructs described herein, as outlined in Example 5.

FIG. 14 is a graph comparing heparan sulfate levels in liver, spleen, kidney, lung and heart tissue samples from untreated MPS-1 mice (grey bars) and MPS-1 mice implanted with encapsulated cells expressing an exemplary anti-HSA-anti-IGF1R-hIDUA fusion enzyme (R28-IGF1R5-hIDUA, black bars).

FIG. 15 is a graph comparing heparan sulfate levels in brain tissue samples from untreated MPS-1 mice (solid black bar) and MPS-1 mice implanted with encapsulated cells expressing an exemplary hIDUA fusion enzyme grey bars).

### DETAILED DESCRIPTION

The present disclosure features BBB-transporting fusion proteins that comprise HSA- and hIGF1R-binding domains and a cargo moiety, mammalian cells (e.g., human RPE cells) genetically modified to express and secrete these fusion proteins, as well as compositions and devices comprising the genetically modified cells. In some embodiments, the devices comprise a cell-containing compartment which includes a cell binding substance as well as the genetically modified cells. In some embodiments, the devices are configured to mitigate the FBR when placed inside a subject, e.g., a human subject. In some embodiments, the fusion proteins, genetically modified cells, compositions, and devices are useful for the treatment of a CNS condition or disorder such as a lysosomal storage disease. Various embodiments will be described below.

### ABBREVIATIONS AND DEFINITIONS

Throughout the detailed description and examples of the disclosure the following abbreviations will be used.
CNS central nervous system
CS chondroitin sulfate
DS dermatan sulfate
GAA acid alpha-glucosidase
GAG glycosaminoglycan
GBA beta-glucosidase
GLA alpha-galactosidase A
GNS N-acetylgalactosamine-6-sulfatase
GUSB beta-glucoronidase
HS heparan sulfate
HGSNAT heparan-alpha-glucosaminide N-acetyltransferase protein
IDS iduronate-2-sulfatase protein
IDUA alpha-L-iduronidase protein
MPS mucopolysaccharidoses
NAGLU alpha-N-acetyl-glucosaminidase
SGSH N-sulfoglucosamine sulfohydrolase

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About” or “approximately” when used herein to modify a numerically defined parameter (e.g., amount of a fusion protein secreted by an engineered cell, a physical description of a device (e.g., hydrogel capsule) such as diameter, sphericity, number of cells encapsulated therein, the number of devices in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a hydrogel capsule defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 devices (e.g., hydrogel capsules) includes preparations having 80 to 120 devices. In some embodiments, the term “about” means that the modified parameter may vary by as much as 15%, 10% or 5% above and below the stated numerical value for that parameter.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer,” “administering,” or “administration,” as used herein, refer to implanting, absorbing, ingesting, injecting, placing, or otherwise introducing into a subject, an entity described herein (e.g., a device or a preparation of devices), or providing such an entity to a subject for administration.

“Afibrotic”, as used herein, means a compound or material that mitigates the foreign body response (FBR). For example, the amount of FBR in a biological tissue that is induced by implant into that tissue of a device (e.g., hydrogel capsule) comprising an afibrotic compound (e.g., a hydrogel capsule comprising a polymer covalently modified with a compound listed in Table 6) is lower than the FBR induced by implantation of an afibrotic-null reference device, i.e., a device that lacks any afibrotic compound, but is of substantially the same composition (e.g., same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to the claimed capsule. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

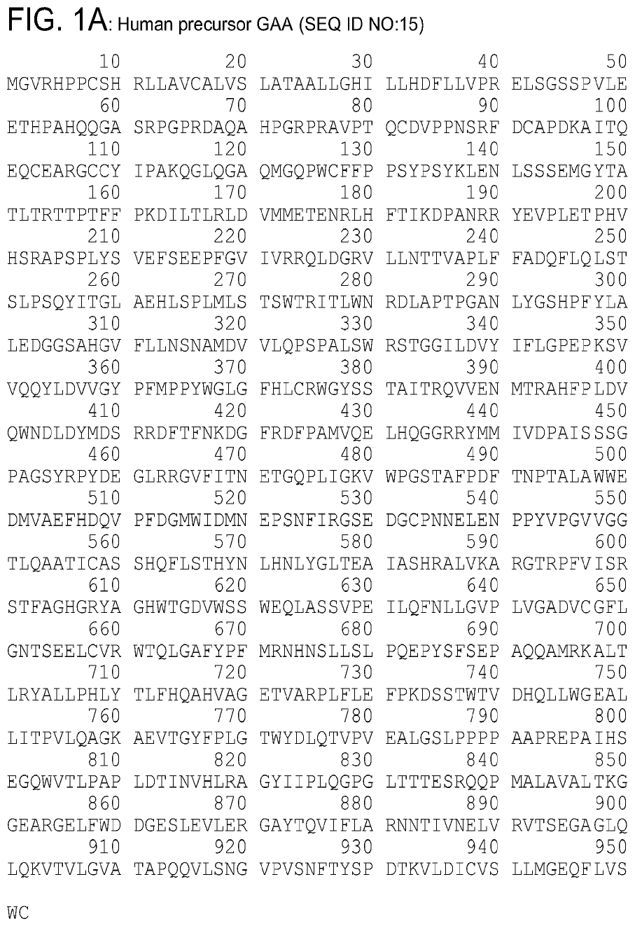

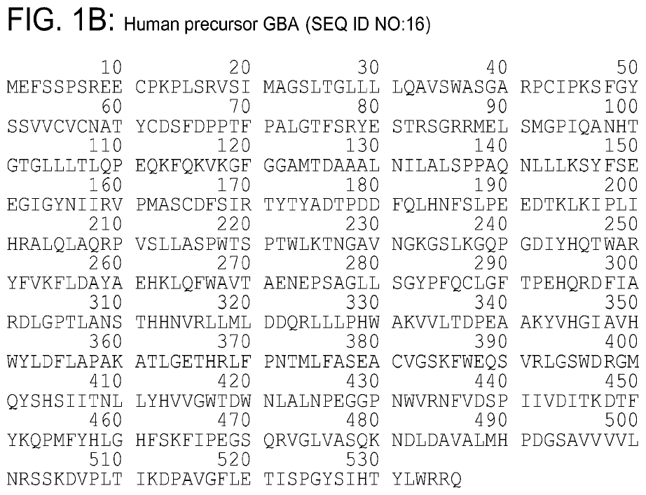

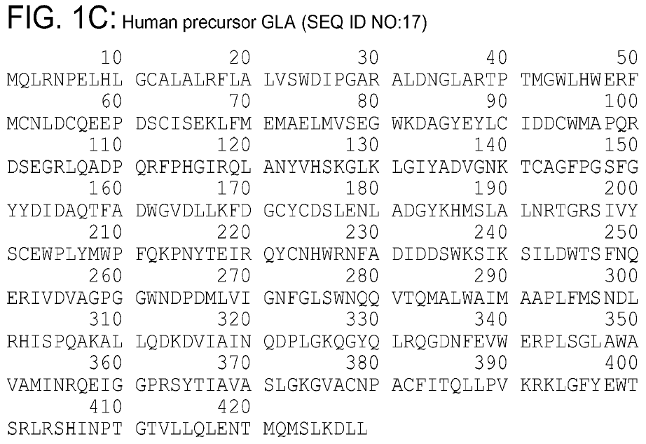

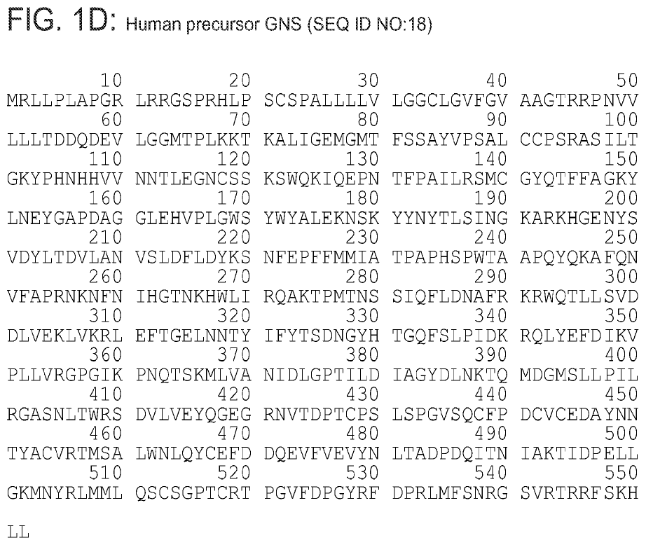

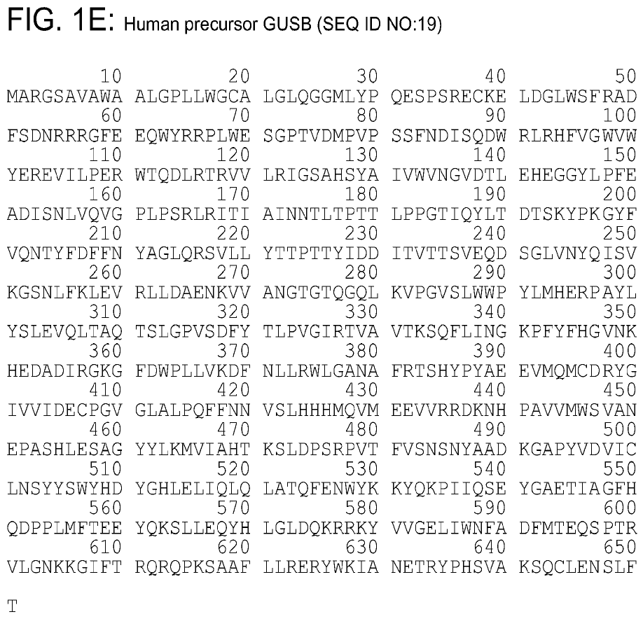

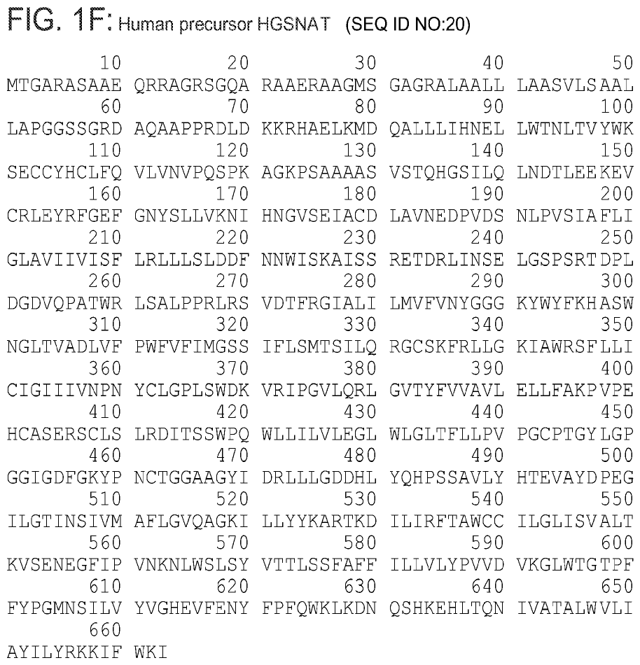

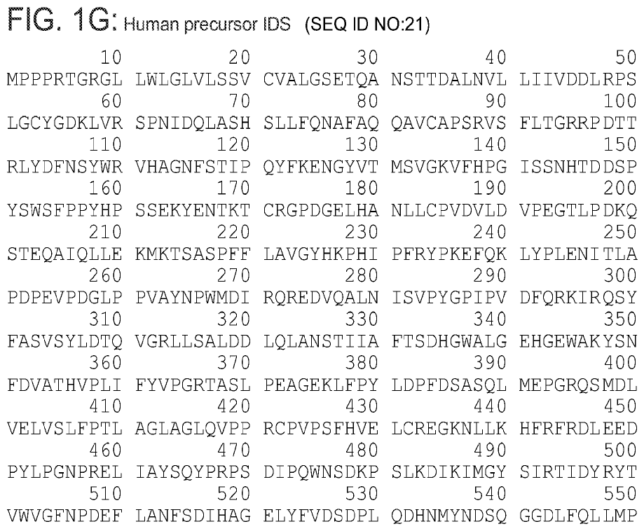

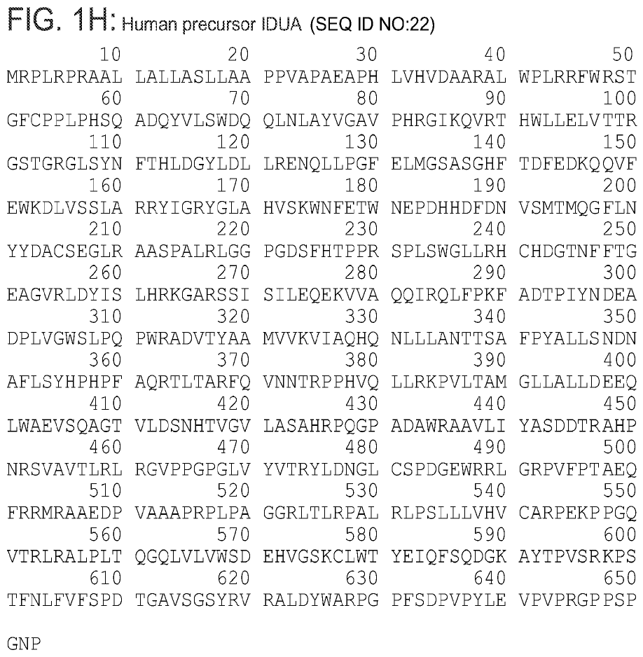

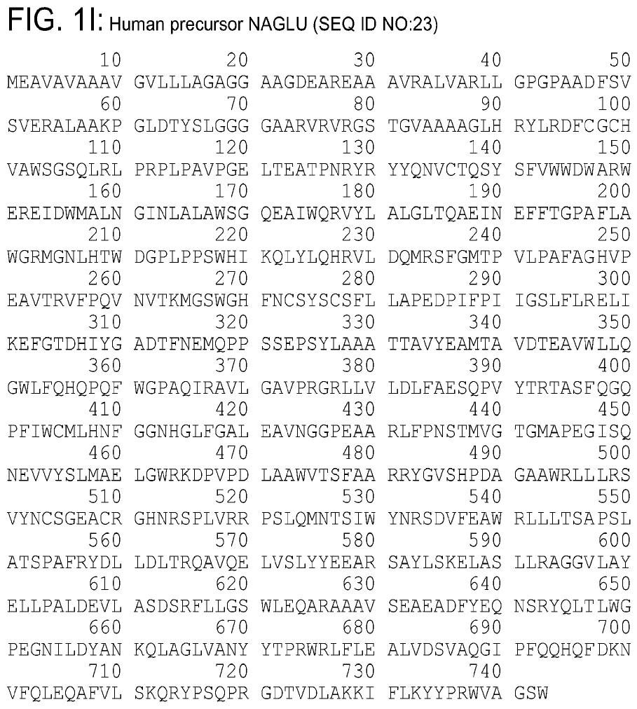

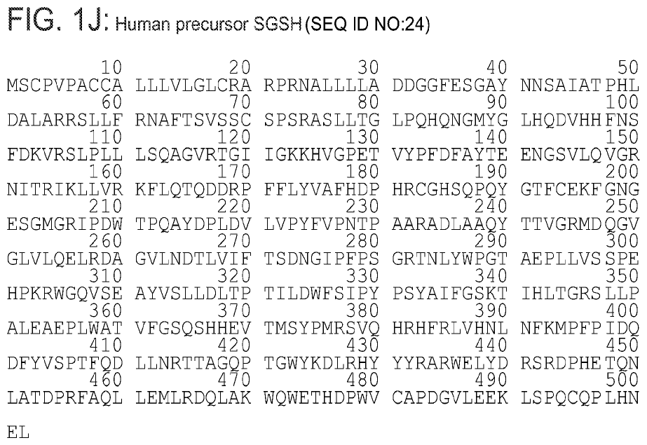

“Acid alpha-glucosidase protein”, “acid maltase protein”, “alpha-1,4-glucosidase protein” and “GAA protein” may be used interchangeably herein and refer to a protein comprising the mature amino acid sequence encoded by a wild-type mammalian (e.g., human) GAA gene or any fragment, mutant, variant or derivative thereof that has GAA enzyme activity that is within 80-120%, 85-115%, 90-110% or 95-105% of the corresponding wild-type mammalian mature GAA protein, as measured by any art-recognized GAA activity assay. The GAA enzyme catalyzes the hydrolysis of alpha(1,4) and alpha(1,6) linkages in glycogen, yielding free glucose and shortened glycogen polymers. GAA enzymatic activity can be measured using any art-recognized assay. The wild-type human GAA gene encodes a 952 amino acid precursor pro-polypeptide, of which the N-terminal 27 amino acids constitute a signal peptide, and amino acids 28-69 constitute a pro-peptide (UniProtKB—P10253). In some embodiments, the GAA amino acid sequence in a BBB-transporting fusion protein described herein comprises amino acids 70-952 of the human precursor GAA sequence shown in FIG. 1A. In an embodiment, the human GAA amino acid sequence in a BBB-transporting fusion protein consists essentially of amino acids 28-952 of the sequence shown in FIG. 1A.

“Alpha-galactosidase A protein”, “α-Gal A protein”, and “GLA protein” may be used interchangeably herein and refer to a protein comprising the mature amino acid sequence encoded by a wild-type mammalian (e.g., human) GLA gene or any fragment, mutant, variant or derivative thereof that has GLA enzyme activity that is within 80-120%, 85-115%, 90-110% or 95-105% of the corresponding wild-type mammalian mature GLA protein, as measured by any art-recognized GLA activity assay. The GLA enzyme hydrolyzes the terminal alpha-D-galactose residues in glycosphingolipids, particularly in globotriaosylceramide (Gb₃). GLA enzymatic activity can be measured using any art-recognized assay. The wild-type human GLA gene encodes a 429-amino acid polypeptide, of which the N-terminal 31 amino acids constitute a signal peptide (UniProtKB—P06280). In some embodiments, the human GLA amino acid sequence in a BBB-transporting fusion protein described herein consists essentially of amino acids 32-429 of human precursor GLA amino acid sequence shown in FIG. 1C.

“Alpha-L-iduronidase protein” and “IDUA protein” may be used interchangeably herein and refer to a protein comprising the mature amino acid sequence encoded by a wild-type mammalian (e.g., human) IDUA gene or any fragment, mutant, variant or derivative thereof that has IDUA enzyme activity that is within 80-120%, 85-115%, 90-110% or 95-105% of the corresponding wild-type mammalian mature IDUA protein, as measured by any art-recognized IDUA activity assay (e.g., hydrolysis of the substrate 4-methylumbelliferyl-α-L-iduronide (4MU-iduronide), see, e.g., Ou, L. et al., Mol Genet Metab. 2014 February: 111(2): 113-115) or any IDUA activity assay described herein. IDUA protein hydrolyzes nonreducing terminal alpha-L-iduronic acid residues in glycosaminoglycans (GAGs) (e.g., dermatan sulfate and heparan sulfate). The wild-type human IDUA gene encodes a 653 amino acid precursor protein, of which the N-terminal 26 or 27 amino acids constitute a signal peptide (GenBAnk Accession No. AAA81589.1, GenBAnk Accession No. AAA51698.1; UniProtKB—P35475). In some embodiments, the mature human IDUA amino acid sequence in a BBB-transporting fusion protein described herein comprises amino acid 26, 27 or 28 to amino acid 653 of the precursor human IDUA amino acid sequence shown in FIG. 1H. In an embodiment, the mature human IDUA amino acid sequence in a BBB-transporting fusion protein consists essentially of amino acids 27-653 of the amino acid sequence shown in FIG. 1H.

“Alpha-N-acetyl-glucosaminidase protein”, N-acetyl-alpha-glucosaminidase” and “NAGLU protein” may be used interchangeably herein to refer to a protein that comprises the mature amino acid sequence encoded by a wild-type mammalian (e.g., human) NAGLU gene or any fragment, mutant, variant or derivative thereof that has enzyme activity that is within 80-120%, 85-115%, 90-110% or 95-105% of the corresponding wild-type mammalian mature NAGLU protein, as measured by any art-recognized NAGLU assay. NAGLU catalyzes the hydrolysis of terminal non-reducing N-acetyl-D-glucosamine residues in N-acetyl-alpha-D-glucosaminides. The wild-type human NAGLU gene encodes a 743 amino acid precursor polypeptide, of which the N-terminal 23 amino acids constitute a signal peptide (UniProtKB—P54802). In some embodiments, the mature human NAGLU amino acid sequence in a BBB-transporting fusion protein described herein consists essentially of amino acids 24-743 of the amino acid sequence shown in FIG. 1I.

“Beta-glucuronidase protein” and “GUSB protein” may be used interchangeably herein to refer to a protein that comprises the mature amino acid sequence encoded by a wild-type mammalian (e.g., human) GUSB gene or any fragment, mutant, variant or derivative thereof that has enzyme activity that is within 80-120%, 85-115%, 90-110% or 95-105% of the corresponding wild-type mammalian mature GUSB protein, as measured by any art-recognized GUSB assay. GUSB catalyzes the hydrolysis of beta-D-glucuronoside into an alcohol and D-glucuronate. The wild-type human GUSB gene encodes a 651 amino acid precursor polypeptide, of which the N-terminal 22 amino acids constitute a signal peptide (UniProtKB P08236). In some embodiments, the mature human GUSB amino acid sequence in a BBB-transporting fusion protein described herein consists essentially of amino acids 23-651 of the sequence shown in FIG. 1E.

“Beta-glucosidase protein”, “acid beta-glucocerebrosidase protein”, “glucocerebrosidase protein”, “glucosylceramidase protein”, “lysosomal acid glucosylceramidase protein”, “lysosomal acid GCase protein”, and “GBA protein” may be used interchangeably herein to refer to a protein that comprises the mature amino acid sequence encoded by a wild-type mammalian (e.g., human) GBA gene or any fragment, mutant, variant or derivative thereof that has enzyme activity that is within 80-120%, 85-115%, 90-110% or 95-105% of the corresponding wild-type mammalian mature GBA protein, as measured by any art-recognized GBA assay. GBA, within the lysosomal compartment, catalyzes the breakdown of the glycolipid glucosylceramide (GicCer) to ceramide and glucose. The wild-type human GBA gene encodes a 536 amino acid precursor polypeptide, of which the N-terminal 39 amino acids constitute a signal peptide (UniProtKB P04062.3). In some embodiments, the mature human GBA amino acid sequence in a BBB-transporting fusion protein described herein consists essentially of amino acids 40-536 of the sequence shown in FIG. 1B.

“Cell,” as used herein, refers to an engineered cell (e.g., a genetically modified cell), or a cell that is not engineered. In an embodiment, a cell is an immortalized cell, or an engineered cell derived from an immortalized cell. In an embodiment, the cell is a live cell, e.g., is viable as measured by any technique described herein or known in the art.

“Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). In an embodiment, the CBP is any of the CBPs described in international patent publication WO2020069429. In an embodiment, the CBP is a linear peptide comprising RGD (SEQ ID NO:87) and is less than 10 amino acids in length. In an embodiment, the CBP is a linear peptide that consists essentially of GRGD (SEQ ID NO:88) or GRGDSP (SEQ ID NO:89).

“CBP-polymer”, as used herein, means a polymer comprising at least one cell-binding peptide molecule covalently attached to the polymer via a linker. In an embodiment, the polymer in a CBP-polymer is a synthetic or naturally-occurring polysaccharide, e.g., an alginate, e.g., a sodium alginate. In an embodiment, the linker is an amino acid linker (i.e., consists essentially of a single amino acid, or a peptide of several identical or different amino acids), which is joined via a peptide bond to the N-terminus or C-terminus of the CBP. In an embodiment, the CBP-polymer is any of the CBP-alginates defined in WO2020069429.

“Cell-binding substance (CBS)”, as used herein, means any chemical, biological, or other type of substance (e.g., a small organic compound, a peptide, a polypeptide) that is capable of mimicking at least one activity of a ligand for a cell-adhesion molecule (CAM) or other cell-surface molecule that mediates cell-matrix junctions or cell-cell junctions or other receptor-mediated signaling. In an embodiment, when present in a polymer composition encapsulating live cells, the CBS is capable of forming a transient or permanent bond or contact with one or more of the cells. In an embodiment, the CBS facilitates interactions between two or more live cells encapsulated in the polymer composition. In an embodiment, the presence of a CBS in a polymer composition encapsulating a plurality of cells (e.g., live cells) is correlated with one or both of increased cell productivity (e.g., expression of a therapeutic agent) and increased cell viability when the encapsulated cells are implanted into a test subject, e.g., a mouse. In an embodiment, the CBS is physically attached to one or more polymer molecules in the polymer composition. In an embodiment, the CBS is a cell-binding peptide, as defined herein or in WO2020069429.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. In some embodiments, a conservatively modified variant of an HSA-binding domain sequence and/or IGF1R binding domain sequence does not include substitutions of any amino acids in the CDRs. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 1 below.

TABLE 1
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/ | Asp, Glu, Asn, Aln, His, Arg, Lys |
| Surface Exposure | Cys, Ser, Tyr, Pro, Ala, Gly, Trp, Tyr |
| | Met, Ile, Leu, Val, Phe |
| Secondary | Ala, Glu, Aln, His, Lys, Met, Leu, Arg |
| Structure Propensity | Cys, Thr, Ile, Val, Phe, Tyr, Trp |
| | Ser, Gly, Pro, Asp, Asn |
| Evolutionary | Asp, Glu |
| Conservation | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, an HSA-binding domain or an IGF1R-binding domain that consists essentially of a recited amino acid sequence may also include one or more amino acids, including substitutions in the recited amino acid sequence, of one or more amino acid residues, which do not materially affect the relevant biological activity of the HSA-binding domain or an IGF1R-binding domain, respectively.

“Derived from”, as used herein with respect to a cell or cells, refers to cells obtained from tissue, cell lines, or cells, which optionally are then cultured, passaged, immortalized, differentiated and/or induced, etc. to produce the derived cell(s).

“Device”, as used herein, refers to any implantable object (e.g., a particle, a hydrogel capsule, an implant, a medical device), which contains an engineered cell or cells (e.g., live cells) capable of expressing and secreting a fusion protein following implant of the device, and has a configuration that supports the viability of the cells by allowing cell nutrients to enter the device.

“Effective amount”, as used herein, refers to an amount of any of the following: genetically-modified cells secreting a BBB-transporting fusion protein, a device preparation producing the fusion protein, number of genetically-modified cells in a device, amount of a CBS and/or afibrotic compound in a device that is sufficient to elicit a desired biological response. In some embodiments, the term “effective amount” refers to the amount of a component of the device (e.g., number of cells in the device, the density of an afibrotic compound disposed on the surface and/or in a barrier compartment of the device, the density of a CBS in the cell-containing compartment.

In an embodiment, the desired biological response is an increase in levels of the cargo molecule (e.g., cargo polypeptide) in a tissue sample removed from a subject treated with (e.g., implanted with) the genetically modified cells, a device or a device preparation containing such cells. As will be appreciated by those of ordinary skill in this art, the effective amount may vary depending on such factors as the desired biological endpoint, the pharmacokinetics of the secreted BBB-transporting fusion protein, composition or device, the condition being treated, the mode of administration, and the age and health of the subject. An effective amount encompasses therapeutic and prophylactic treatment.

In an embodiment, an effective amount of a compound of Formula (III) disposed on or in a device is an amount that reduces the FBR to the implanted device compared to a reference device, e.g., reduces fibrosis or amount of fibrotic tissue on or near the implanted device.

In an embodiment, an effective amount of a CBS disposed with engineered cells in a cell-containing compartment is an amount that enhances the viability of the cells (e.g., number of live cells) compared to a reference device and/or increases the production of the BBB-transporting fusion protein by the cells (e.g., increased levels of the fusion protein in plasma of a subject implanted with the device) compared to a reference device. An effective amount of a device, composition or component (e.g., afibrotic compound, CBS, engineered cells) may be determined by any technique known in the art of described herein.

“Engineered cell” or “genetically-modified cell,” as used herein, is a mammalian cell (e.g., a human cell, e.g., an RPE cell, a cell derived from a cell line, (e.g., ARPE-19 or other cell line), a stem cell, a cell differentiated from an iPSC) having a non-naturally occurring alteration, and typically comprises an exogenous nucleotide sequence (e.g., a vector or an altered chromosomal sequence), encoding a BBB-transporting fusion protein described herein. In an embodiment, the exogenous nucleotide sequence is chromosomal (e.g., the exogenous sequence is disposed in endogenous chromosomal sequence) or is extra chromosomal (e.g., a non-integrated expression vector). In an embodiment, the exogenous nucleotide sequence in a genetically modified cell comprises a codon optimized coding sequence for one, two or all three of the AB, BBB or C domains that achieves higher expression of the fusion protein than a naturally-occurring coding sequence for each of these domains. The codon optimized sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, www.jcat.de, Grote, A. et al., Nucleic Acids Research, Vol 33, Issue suppl_2, pp. W526-W531 (2005). In an embodiment, the cell is also genetically modified to reduce or eliminate expression of one or more proteins naturally expressed by the parental cell. In an embodiment, a genetically modified cell (e.g., modified RPE cell, a modified ARPE-19 cell) is cultured from a population of stably-transfected cells, or from a monoclonal cell line.

An “exogenous nucleotide sequence,” as used herein, is a nucleotide sequence that does not occur naturally in a subject cell.

An “exogenous polypeptide,” as used herein, is a polypeptide that does not occur naturally in a subject cell, e.g., engineered cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Expression vector”, as used herein, refers to a recombinant polynucleotide comprising one or more expression constructs encoding one or more proteins to be expressed. Each expression construct contains expression control sequences operatively linked to one or more nucleotide sequences to be expressed. An expression vector comprises sufficient cis-acting elements for expression; other elements for expression can be supplied by the host cell or in an in vitro expression system. The vector may comprise additional sequence elements used for the expression of and/or the integration of the expression cassette(s) into the genome of a mammalian cell. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. The expression vectors suitable for use in engineering mammalian cells to express any of the fusion proteins described herein may also contain a nucleotide sequence encoding a marker for selection of cells that contain such a vector. Examples of a suitable marker are genes that encode resistance to antibiotics, such as ampicillin, chloramphenicol, kanamycin, nourseothricin, or zeocin.

“Fabry disease”, “GLA deficiency”, “alpha-galactosidase A deficiency”, and “Anderson-Fabry disease”, may be used interchangeably herein, to refer to an X-linked, LSD caused by deficient activity of the enzyme alpha-galactosidase A (GLA or GALA), which leads to damaging accumulation of the glycosphingolipid globotriaosylceramide (Gb3) in various tissues and organs. More than 370 different mutations in the human GLA gene have been identified in people with Fabry disease, many of which are unique to single families. Mutations that eliminate GLA activity lead to the severe, classic form of Fabry disease, which typically begins in childhood. Milder, late-onset forms of Fabry disease are correlated with mutations that reduce, but do not eliminate, GLA activity. Fabry patient refers to an individual who has been diagnosed with or suspected of having Fabry disease. A Fabry patient may be diagnosed using any method known in the art. In an embodiment, a human Fabry disease patient has a GLA gene mutation associated with deficient GLA enzymatic activity and/or Fabry disease.

“Gaucher disease”, as used herein, refers to an autosomal recessive LSD caused by deficient activity of beta-glucocerebrosidase, which leads to intracellular accumulation of the glycolipid glucocerebroside throughout the body. At least five forms of Gaucher disease have been identified; the forms associated with CNS complications are named Gaucher disease type 2 and Gaucher disease type 3. The type 2 form, also known as acute neuronopathic Gaucher disease, occurs in newborns and infants, who typically die within the first three years of life. The type 3 form, also known as chronic neuronopathic Gaucher disease, typically occurs during the first decade of life, with CNS complications that develop and progress slower than in Type 2 patients. Numerous mutations in the GBA gene have been linked to type 2 and type 3 Gaucher disease, with an L483P substitution being the most common mutation associated with type 3. Gaucher patient refers to an individual who has been diagnosed with or suspected of having Gaucher disease. In an embodiment, a human Gaucher patient has a GBA gene mutation associated with deficient GBA enzymatic activity and/or Gaucher disease type 3.

“Heparan-alpha-glucosaminide N-acetyltransferase protein”, “heparan acetyl-CoA:alpha-glucosaminide N-acetyltransferase protein”, “HGSNAT protein”, and “N-acetyltransferase protein” may be used interchangeably herein to a protein that comprises the mature amino acid sequence encoded by a wild-type mammalian (e.g., human) gene or any fragment, mutant, variant or derivative thereof that has HGSNAT enzyme activity that is within 80-120%, 85-115%, 90-110% or 95-105% of the corresponding wild-type mammalian mature HGSNAT protein, as measured by any art-recognized HGSNAT assay. HGSNAT catalyzes acetylation of the terminal glucosamine residues of intralysosomal heparan or heparan sulfate, converting it into a substrate for hydrolysis by NAGLU. The wild-type human HGSNAT gene encodes a 663 amino acid polypeptide, which includes a predicted signal sequence that is not cleaved upon translocation into the endoplasmic reticulum (UniProtKB—Q68CP4). In some embodiments, the HGSNAT amino acid sequence in a BBB-transporting fusion protein described herein consists essentially of amino acids 1-663 of the human HGSNAT sequence shown in FIG. 1F.

“High molecular weight alginate” or “HMW-Alg”, as used herein, means an alginate with an approximate molecular weight of 150 kDa-250 kDa.

“Iduronate-2-sulfatase protein”, “IDS protein”, “I2S protein”, and “Alpha-L-iduronate sulfate sulfatase protein” may be used interchangeably herein to refer to a protein that comprises the mature amino acid sequence encoded by a wild-type mammalian (e.g., human) IDS gene or any fragment, mutant, variant or derivative thereof that has IDS enzyme activity that is within 80-120%, 85-115%, 90-110% or 95-105% of the corresponding wild-type mammalian mature IDS protein, as measured by any art-recognized IDS assay. IDS hydrolyzes the 2-sulfate groups of the L-iduronate 2-sulfate units of dermatan sulfate, heparan sulfate and heparan. The wild-type human IDS gene encodes a 550 amino acid precursor pro-polypeptide, of which the N-terminal 25 amino acids constitute a signal peptide, and the remaining amino acids constitute a pro-polypeptide that is processed into the mature polypeptide by removal of the pro-peptide of amino acids 26-33 and then cleavage into two chains formed by amino acids 34-455 and amino acids 456-550. (UniProtKB—P22304). In some embodiments, the IDS amino acid sequence in a BBB-transporting fusion protein described herein comprises amino acids 34-550 of the human precursor IDS sequence shown in FIG. 1G. In an embodiment, the human IDS amino acid sequence in a BBB-transporting fusion protein consists essentially of amino acids 26-550 of the sequence shown in FIG. 1G.

“Low molecular weight alginate” or “LMS-Alg” as used herein, means an alginate with an approximate molecular weight of <75 kDa.

“Lysosomal Storage Disorder” and “LSD”, as used herein, refer to an inborn error of metabolism caused by deficiency of an enzyme or its co-factor that results in defective functioning of lysosomes and the accumulation of one or more glycosaminoglycans (GAGs) in various tissues and organs. GAGs, which are long unbranched polysaccharides consisting of repeating disaccharides, include: chondroitin sulfate (CS); dermatan sulfate (DS), heparan sulfate (HS), keratan sulfate (KS) and hyaluronan. A defective enzyme is the cause of about 70% of LSDs, while the remainder are due to defects in an enzyme activator or associated protein. Known LSDs include the enzyme-deficiency LSDs and other LSDs listed in Pastores, et al., Neurol Clin. 31(4);1051-1071 (2013) at Table 1 and Table 2. In an embodiment, the LSD is a mucopolysaccharidoses (MPS) or a sphingolipidoses (SP).

“Medium molecular weight alginate” or “MMW-Alg” as used herein, means an alginate with an approximate molecular weight of 75 kDa to 150 kDa.

“Mucopolysaccharidoses” and “MPS”, as used herein, refer to a condition caused by deficiency of an enzyme involved in metabolism of glycosaminoglycans that leads to accumulation of glycosaminoglycan fragments in lysosomes and can result in bone, soft tissue, and CNS manifestations, particularly in neuronopathic forms of MPS. The neuronal damage in neuronopathic MPS is related to the storage of undegraded heparan sulfate (HS), and secondary toxic products such as GM2 and GM3 gangliosides, inflammatory cytokines, and reactive oxygen species. Neuronopathic MPS includes MPS-1 Hurler (MPS-1H), MPS-2, MPS-3(A-D) and MPS-7.

“Mucopolysaccharidosis type I” and “MPS 1” may be used interchangeably herein to refer to an LSD caused by deficient IDUA enzymatic activity and consequent accumulation of GAGs (primarily DS and HS) within lysosomes in multiple organs and tissues. MPS 1 patient refers to an individual (e.g., a human) who has been diagnosed with or suspected of having MPS 1 disease, e.g., severe MPS 1 or attenuated MPS 1. The patient may be diagnosed using any method known in the art, including clinical, biochemical and genetic methods for diagnosing MPS 1. In an embodiment, a human MPS 1 patient has a mutation in the IDUA gene that is associated with deficient IDUA enzymatic activity and/or MPS 1 disease.

“Mucopolysaccharidosis type II”, “MPS 2” and “Hunter syndrome” may be used interchangeably herein to refer to an X-linked LSD caused by deficient IDS enzymatic activity and consequent accumulation of GAGs (primarily DS and HS) within lysosomes in multiple organs and tissues. MPS 2 patient refers to an individual (e.g., a human) who has been diagnosed with or suspected of having MPS 2 disease, e.g., severe early-onset MPS 2 (symptoms become apparent within 2-4 years of age) or mild, late-onset MPS 2. The patient may be diagnosed using any method known in the art, including clinical, biochemical and genetic methods for diagnosing MPS 2. In an embodiment, a human MPS 2 patient has a mutation in the IDS gene that is associated with deficient IDS activity and/or MPS 2 disease.

“Mucopolysaccharidosis type IIIA”, “MPS 3A” and “Sanfilippo syndrome type A” may be used interchangeably herein to refer to an autosomal recessive LSD caused by deficient SGSH activity and consequent accumulation of heparan sulfate in the CNS. MPS 3A is characterized by severe CNS degeneration. More than 80 different mutations in the SGSH gene have been identified in MPS 3A patients. MPS 3A patient refers to an individual (e.g., a human) who has been diagnosed with or suspected of having MPS3A disease. The patient may be diagnosed using any method known in the art, including clinical, biochemical and genetic methods for diagnosing MPS 3A. In an embodiment, a human MPS 3A patient has a SGSH gene mutation associated with deficient SGSH enzymatic activity and/or MPS 3A disease.

“Mucopolysaccharidosis type IIIB”, “MPS 3B” and “Sanfilippo syndrome type B” may be used interchangeably herein to refer to an autosomal recessive LSD caused by deficient NAGLU enzyme activity and consequent accumulation of HS in the CNS. More than 100 different mutations in the human NAGLU gene have been associated with the MPS 3B phenotype. MPS 3B patient refers to an individual (e.g., a human) who has been diagnosed with or suspected of having MPS 3B disease. The patient may be diagnosed using any method known in the art, including clinical, biochemical and genetic methods for diagnosing MPS 3B. In an embodiment, a human MPS 3B patient has a NAGLU gene mutation associated with deficient NAGLU enzymatic activity and/or MPS 3B disease.

“Mucopolysaccharidosis type IIIC”, “MPS 3C” and “Sanfilippo syndrome type C” may be used interchangeably herein to refer to an autosomal recessive LSD caused by deficient HGSNAT enzyme activity and consequent accumulation of heparan sulfate in the CNS. MPS 3C disease onset is typically before age 10 years and is characterized by progressive CNS degeneration. More than 50 different mutations in the HGSNAT gene have been identified in MPS 3C patients. MPS 3C patient refers to an individual (e.g., a human) who has been diagnosed with or suspected of having MPS 3C disease. The patient may be diagnosed using any method known in the art, including clinical, biochemical and genetic methods for diagnosing MPS 3C. In an embodiment, a human MPS 3C patient has an HGSNAT gene mutation associated with deficient HGSNAT enzymatic activity and/or MPS 3C disease.

“Mucopolysaccharidosis type IIID”, “MPS 3D”, and “Sanfilippo syndrome type D” may be used interchangeably herein to refer to an autosomal recessive LSD caused by deficient GNS enzyme activity and consequent accumulation of HS in the CNS. MPS 3D disease onset is typically between ages 2 and 6 years and is characterized by severe neurological degeneration in most patients between 6 and 10 years of age. MPS 3D patient refers to an individual (e.g., a human) who has been diagnosed with or suspected of having MPS 3D disease. The patient may be diagnosed using any method known in the art, including clinical, biochemical and genetic methods for diagnosing MPS-3A. In an embodiment, a human MPS 3D patient has a GNS gene mutation associated with deficient GNS enzymatic activity and/or MPS 3D disease.

“Mucopolysaccharidosis type VII”. “MPS7” and Sly syndrome” may be used interchangeably herein to refer to an autosomal recessive LSD caused by deficient GUSB enzyme activity and consequent accumulation of CS, DS and HS in the lysosome of multiple tissues. At least 49 different mutations in the GUSB gene have been identified in MPS 7 patients. MPS 7 patient refers to an individual (e.g., a human) who has been diagnosed with or suspected of having MPS 7 disease. The patient may be diagnosed using any method known in the art, including clinical, biochemical and genetic methods for diagnosing MPS 7. In an embodiment, a human MPS 7 patient has a GUSB gene mutation associated with deficient GUSB enzymatic activity and/or MPS 7 disease.

“N-acetylgalactosamine-6-sulfatase protein”, “Glucosamine N-acetyl-6-sulfatase protein” and “GNS protein” may be used interchangeably herein to a protein that comprises the mature amino acid sequence encoded by a wild-type mammalian (e.g., human) GNS gene or any fragment, mutant, variant or derivative thereof that has GNS enzyme activity that is within 80-120%, 85-115%, 90-110% or 95-105% of the corresponding wild-type mammalian mature GNS protein, as measured by any art-recognized GNS enzymatic assay. GNS catalyzes the hydrolysis of the 6-sulfate groups of the N-acetyl-D-glucosamine 6-sulfate units of heparan sulfate and keratan sulfate. The wild-type human GNS gene encodes a 552 amino acid precursor polypeptide, of which the N-terminal 36 amino acids constitute a signal peptide (UniProtKB—P15586). In some embodiments, the mature human GNS amino acid sequence in a BBB-transporting fusion protein described herein consists essentially of amino acids 37-552 of the human precursor GNS sequence shown in FIG. 1D.

“N-sulfoglucosamine sulfohydrolase”, “SGSH”, “Sulfamidase” and “heparan-N-sulfatase” may be used interchangeably herein to refer to a protein that comprises the mature amino acid sequence encoded by a wild-type mammalian (e.g., human) SGSH gene or any fragment, mutant, variant or derivative thereof that has enzyme activity that is within 80-120%, 85-115%, 90-110% or 95-105% of the corresponding wild-type mammalian mature SGSH protein, as measured by any art-recognized SGSH assay. SGSH catalyzes the hydrolysis of N-sulfo-D-glucosamine into D-glucosamine and sulfate. The wild-type human SGSH gene encodes a 502 amino acid precursor polypeptide, of which the N-terminal 20 amino acids constitute a signal peptide (UniProtKB—P51688). In some embodiments, the mature human SGSH amino acid sequence in a BBB-transporting fusion protein described herein consists essentially of amino acids 21-502 of the sequence shown in FIG. 1J.

“Peptide”, as used herein, is a polypeptide of less than 50 amino acids, typically, less than 25 amino acids.

“PolyA signal”, as used herein, refers to any continuous sequence that terminates transcription of a coding sequence into RNA and directs addition of a polyA tail onto the RNA. Examples of polyA signals are the rabbit binding globulin (rBG) polyA signal, the SV40 late poly A signal, the SV50 polyA signal, the bovine growth hormone (BGH) poly A signal, the human growth hormone (HGH) polyA signal and synthetic polyA signals known in the art.

“Polymer composition”, as used herein, is a composition (e.g., a solution, mixture) comprising one or more polymers. As a class, “polymers' includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, is a polymer comprising amino acid residues linked through peptide bonds and having at least two, and in some embodiments, at least 10, 50, 75, 100, 150 or 200 amino acid residues.

“Pompe disease (PD)”, “acid alpha-glucosidase deficiency”, and “glycogen storage disease type II” may be used interchangeably herein to refer to an autosomal recessive LSD caused by deficient GAA enzymatic activity and consequent excessive accumulation of lysosomal glycogen primarily in the heart, skeletal, smooth muscles, and the nervous system. PD is broadly classified into infantile PD (IPD) and late-onset PD (LOPD). More than 580 mutations in the GAA gene have been identified in PD patients. PD patient refers to an individual (e.g., a human) who has been diagnosed with or suspected of having PD. The patient may be diagnosed using any method known in the art, including clinical, biochemical and genetic methods for diagnosing PD. In an embodiment, a human PD patient has a GAA gene mutation associated with deficient GAA enzymatic activity and/or PD. In an embodiment, the patient has been diagnosed with LOPD.

“Prevention,” “prevent,” and “preventing”, as used herein, refer to a treatment that comprises administering or applying a BBB-transporting fusion protein described herein, e.g., administering a composition of devices encapsulating modified cells expressing the fusion protein (e.g., as described herein), prior to the onset of one or more symptoms of a CNS condition or disease to preclude the physical manifestation of the symptom(s). In some embodiments, “prevention,” “prevent,” and “preventing” require that signs or symptoms of the CNS condition/disease have not yet developed or have not yet been observed. In some embodiments, treatment comprises prevention and in other embodiments it does not.

“Promoter sequence”, as used herein refers to a nucleotide sequence that is capable of driving expression in a mammalian cell, e.g., a human cell, e.g., an RPE cell, e.g., an ARPE-19 cell. In some embodiments, e.g., for driving expression of a BBB-transporting fusion protein described herein, the promoter sequence is from a strong mammalian promoter, e.g., a human promoter sequence. Non-limiting examples of strong promoters for use in expression cassettes described herein include the EF1A promoter, CAG promoter, PGK (phosphoglycerate kinase) promoter and the ACTB (human beta-actin) promoter. In an embodiment, a promoter sequence useful for driving expression of a ST protein described herein may be from a medium-strength promoter, e.g., the EFS promoter sequence, which is a shortened form of the EF1A promoter sequence.

“RPE cell” as used herein refers to a cell having one or more of the following characteristics: a) it comprises a retinal pigment epithelial cell (RPE) (e.g., cultured using the ARPE-19 cell line (ATCC® CRL-2302™)) or a cell derived or modified therefrom, e.g., by stably transfecting cells cultured from the ARPE-19 cell line with an exogenous sequence that encodes a BBB-transporting fusion protein), a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BESTi, or αB-crystallin; iii) it is naturally found in the retina and forms a monolayer above the choroidal blood vessels in the Bruch's membrane; or iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC® CRL-2302™)). In an embodiment, an RPE described herein is genetically modified, e.g., to have a new property, e.g., the cell is modified to express and secrete a fusion protein described herein. In an embodiment, the cell is also genetically modified to reduce or eliminate expression of one or more proteins naturally expressed by the parental cell. In other embodiments, an RPE cell is not genetically modified.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455 A1. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein, mean a device (e.g., a hydrogel capsule or other particle) having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Sphingolipidoses” and “SP”, as used herein, refer to a deficiency of an enzyme involved in the metabolism of sphingolipids that causes sphingolipid accumulation in lysosomes and can lead to manifestations in various organs and tissues, including in visceral and neurological systems. SP diseases include Gaucher disease and Fabry disease.

“Subject” as used herein refers to a human or non-human animal. In an embodiment, the subject is a human (i.e., a male or female) of any age group, e.g., a pediatric human subject (e.g., infant, child, adolescent) or adult human subject (e.g., young adult, middle-aged adult, or senior adult)). In an embodiment, the subject is a non-human animal, for example, a mammal (e.g., a mouse, a dog, a primate (e.g., a cynomolgus monkey or a rhesus monkey). In an embodiment, the subject is a commercially relevant mammal (e.g., cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Treatment,” “treat,” and “treating” as used herein refers to one or more of reducing, reversing, alleviating, delaying the onset of, or inhibiting the progress of one or more of a symptom, manifestation, or underlying cause, of a CNS condition or disease. In an embodiment, treating comprises increasing the activity of a therapeutic protein in the CNS. In an embodiment, treating comprises reducing, reversing, alleviating, delaying the onset of, or inhibiting the progress of a symptom associated with the condition or disease. In some embodiments, “treatment,” “treat,” and “treating” require that signs or symptoms associated with the CNS condition/disease have developed or have been observed. In other embodiments, treatment may be administered in the absence of signs or symptoms of disease, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. In some embodiments, treatment comprises prevention and in other embodiments it does not.

“Wild-type” (wt) refers to the natural form, including sequence, of a polynucleotide, polypeptide or protein in a species. A wild-type form is distinguished from a mutant form of a polynucleotide, polypeptide or protein arising from genetic mutation(s).

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents, e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂—C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₅ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR^{7Q}R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (III) and pharmaceutically acceptable salts thereof described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (III) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds of Formula (III) used to prepare devices of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the devices of the present disclosure (e.g., a particle, a hydrogel capsule) contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

Devices of the present disclosure may contain a compound of Formula (III) in a prodrug form. Prodrugs are those compounds that readily undergo chemical changes under physiological conditions to provide the compounds useful for preparing devices in the present disclosure. Additionally, prodrugs can be converted to useful compounds of Formula (III) by chemical or biochemical methods in an ex vivo environment.

Certain compounds of Formula (III) described herein can exist in unsolvated forms as well as solvated forms, including hydrated forms. In general, the solvated forms are equivalent to unsolvated forms and are encompassed within the scope of the present disclosure. Certain compounds of Formula (III) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

The symbol “ [symbol] ” as used herein refers to a connection to an entity, e.g., a polymer (e.g., [symbol] hydrogel-forming polymer such as alginate) or surface of an implantable device, e.g., a particle, a hydrogel capsule. The connection represented by “ [symbol] ” may refer to direct attachment to the [symbol] entity, e.g., a polymer or an implantable element, may refer to linkage to the entity through an attachment group. An “attachment group,” as described herein, refers to a moiety for linkage of a compound of Formula (III) to an entity (e.g., a polymer or an implantable element (e.g., a device) as described herein), and may comprise any attachment chemistry known in the art. A listing of exemplary attachment groups is outlined in Bioconjugate Techniques (3_{rd} ed, Greg T. Hermanson, Waltham, MA: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —N^{C}N—, —C(═N(R^{C})R^{D}))O—, —S—, —S(O)ₓ—, —OS(O)ₓ—, —N(R^{C})S(O)ₓ—, —S(O)_{X}N(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl. In some embodiments, an attachment group is a cross-linker. In some embodiments, the attachment group is —C(O)(C₁-C₆¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Features of BBB-Transporting Fusion Proteins

BBB-transporting fusion proteins of the disclosure comprise an HSA binding domain (AB) and an IGF1R binding domain (RB) located upstream of a cargo moiety (C) (e.g., amino acid sequence of a therapeutic protein). In some embodiments, a first linker moiety (e.g., a linker peptide) is located between AB and RB and a second linker moiety (e.g., a linker peptide) is located between RB and the cargo moiety.

### HSA-Binding Domain AB

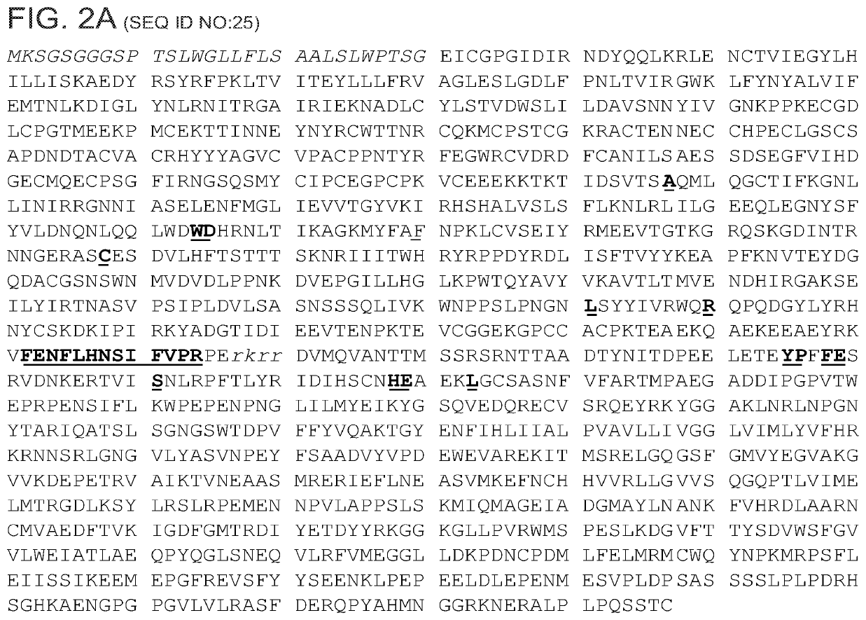

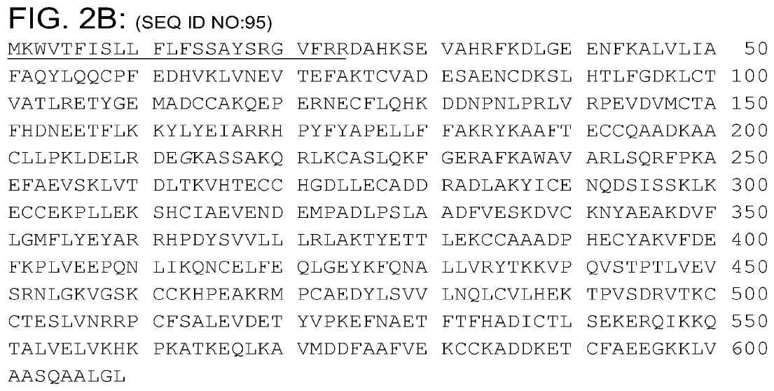

The serum half-life of the BBB-transporting protein is longer than the serum half-life of an otherwise identical fusion protein without the AB domain. This half-life extension is largely due to binding of the fusion protein via AB to HSA. Mature human serum albumin (HSA) is a monomeric protein with 585 amino acids (GenBank Accession No. AAA98797.1), and contains three homologous domains DI (amino acids 25-221 of FIG. 2B); DII (amino acids 213-409 of FIG. 2B); and DIII (amino acids 405-609 of FIG. 2B) (Dockal, M., et al., J. Biol. Chem., Vol. 274, No. 41, pp. 29303-29310 (1999). The long half-life of albumin in blood is mainly driven by two characteristics: (i) the large size (65 kDa) of albumin limits its glomerular filtration and (ii) albumin binds to FcRn at low pH (pH 6), which protects albumin from degradation in the lysosomes after passive endocytosis in endothelial and epithelial cells, by recycling from early endosome back to the extracellular environment. In an embodiment, the AB domain confers upon the BBB-transporting fusion protein a serum half-life in man (expressed as t1/2-beta) that is more than any of 6 hours, 12 hours, 24 hours, 72 hours, one week, two weeks or up to the half-life of HSA in man (estimated to be around 19 days).

In some embodiments, the AB domain is specific for serum albumin, i.e., it does not substantially bind to any non-albumin proteins. In some embodiments, the fusion protein does not substantially inhibit binding of FcRn to HSA, the binding site of which is in DIII. In an embodiment, less than about 20%, 15%, 10%, 5% or 1% inhibition of FcRn binding to HSA occurs in the presence of a fusion protein of the disclosure, which may be determined by any competition binding assay known in the art, e.g., the SPR competition binding assay described in WO 2019/204925 at [0095] or in Example 3 of US 2019/0367596A1. The potential for interference with FcRn binding can be reduced by deriving the AB domain from an anti-HSA antibody that does not binds to DIII. In an embodiment, the fusion protein binds via the AB domain to DI of HSA. In an embodiment, the fusion protein cross-competes via AB for HSA binding with a sdAb that consists of the amino acid sequence for a single domain antibody (sdAb) described in WO2019/204925, i.e., R11, R28, M75 or M79.

In some embodiments, AB (as part of the fusion protein) also binds to (e.g., cross-reacts with) serum albumin from one, two, three, four or more other mammalian species, e.g., any combination of two, three, four or more of mouse, rat, guinea pig, hamster, rabbit, cat, dog, pig, sheep, horse, cow and monkey (e.g., rhesus and/or cynomolgus). In an embodiment, the fusion protein binds via the AB domain to serum albumin from at least mouse, rat, monkey (rhesus or cynomolgus) and human. In an embodiment, the fusion protein binds via the AB domain to serum albumin from at least mouse, dog and human.

In some embodiments, the AB domain (as part of the fusion protein) binds to HSA, and optionally to at least one other mammalian serum albumin, with a desired affinity within a pH range of between about 5.0 or about 5.5 up to about 7.4. In an embodiment, the desired affinity is a dissociation constant (K_{D}) ranging from any of about 0.1 nM to about 1,000 nM, about 0.5 nM to about 500 nM, about 1 nM to about 250 nM, about 5 nM to about 50 nM, about 10 nM to about 25 nM, or about 0.5 nM to about 1 nM. In an embodiment, the affinity is determined by surface plasmon resonance (SPR) at 25° C. and a pH range of about 5.5 to about 7.4. In an embodiment, the affinity of the fusion protein for serum albumin from mouse, rat and monkey is similar to the affinity for HSA, e.g., within 70% to 130%, 80% to 120%, or 90% to 110% of the K_{D} for HSA.

An exemplary SPR assay for measuring K_{D} of the anti-HSA antibody and the fusion protein is described in WO 2019/204925 at [0092]. Another exemplary SPR assay for measuring affinities within the pH range of 5.0 to 7.4 is described in US 2019/0367597A1. The K_{D} of the anti-HSA antibody and the fusion protein for HSA and mammalian orthologues may also be determined substantially as described in WO 2019/246003 at [0994]-[0995].

The AB domain of a BBB-transporting fusion protein of the disclosure comprises the amino acid sequence of a heavy chain variable region (HCVR) of an anti-HSA antibody. To facilitate expression of the fusion protein by genetically-modified cells, in some embodiments, the AB domain has a molecular weight of less than about any of 75 kDa, 50 kDa or 25 kDa. The AB domain may be derived from any anti-HSA antibody molecule known in the art, including conventional 4-chain antibodies, antigen-binding fragments, Fab, Fab′, F(ab′)₂, Fv (double-chain and single-chain (scFv)), minibody, diabody and sdAb. In an embodiment, AB consists essentially of, or consists of, the HCVR amino acid sequence of a sdAb.

In an embodiment, the AB domain comprises the set of three CDR amino acid sequences in one of the anti-HSA sdAbs described in Table I of WO 2006/122787. In an embodiment, AB consists essentially of, or consists of, the amino acid sequence in one of the anti-HSA sdAbs described in Table II of WO 2006/22787 (e.g., Alb-1), or in one of the humanized variants of Alb-1 described in Table III of WO 2006/22787 (e.g., Alb-8). In an embodiment, AB consists essentially of, or consists of, any of the Alb-23 sequences described in WO 2012/175400 (e.g., Alb-23D). In an embodiment, a BBB-transporting fusion protein of the present disclosure cross-competes for binding to HSA with any of the anti-HSA sdAbs described in WO 2006/22787 (e.g., Alb-8) or in WO 2012/175400 (e.g., Alb-23D). In an embodiment, the BBB-transporting fusion protein does not comprise the amino acid sequence for Alb-1. In an embodiment, the BBB-transporting fusion protein does not comprise the amino acid sequence for Alb-8.

In another embodiment, the AB domain consists essentially of, or consists of, the amino acid sequence of an albumin binding domain (ABD) described in Table 7 of WO 2019/246003, e.g., LAEAKVLANRELDKYGVSDYYKNLINNAKTVEGVKALIDEILAALP (SEQ ID NO:40), which is described in WO 2019/246003 as an ABD having a K_{D} to HSA of about 1.2 nM. In an embodiment, the AB domain comprises the three CDRs of the anti-HSA P367 antibody described in Table 14 of WO 2019/2460003. In an embodiment, AB consists essentially of, or consists of, the amino acid sequence of the anti-HSA P367 antibody or its humanized variant P494, each of which is listed in Table 14 of WO 2019/2460003. In an embodiment, a BBB-transporting fusion protein of the present disclosure cross-competes for binding to HSA with the P494 sdAb described in WO 2019/2460003.

In another embodiment, the AB domain comprises a set of three heavy chain CDR amino acid sequences in one of the anti-HSA sdAbs described in Table 5 of WO 2021/119551. In an embodiment, AB consists essentially of, or consists of, the VH amino acid sequence in one of the anti-HSA sdAbs described in Table 5 of WO 2021/119551. In an embodiment, a BBB-transporting fusion protein of the present disclosure cross-competes for binding to HSA with one or more of the sdAbs described in Table 5 of WO 2021/119551.

In another embodiment, the AB domain comprises a set of the three CDR amino acid sequences that are in the T0235002C06 sdAb described in Table B of US 20190367597A1. In an embodiment, AB consists essentially of, or consists of, the amino acid sequence of T0235002C06 described in Table B of US 2019/0367597A1. In an embodiment, a BBB-transporting fusion protein of the present disclosure cross-competes for binding to HSA with the T0235002C06 sdAb described in Table B of US 20190367597A1.

In another embodiment, the AB domain comprises a set of the three CDR amino acid sequences that are in the T0235005D04 sdAb described in Table B of US 20190367596A1. In an embodiment, AB consists essentially of, or consists of, the amino acid sequence of T0235005D04 described in Table B of US 2019/0367596A1. In an embodiment, a BBB-transporting fusion protein of the present disclosure cross-competes for binding to HSA with the T0235002D04 sdAb described in Table B of US 20190367596A1.

In yet another embodiment, the AB domain comprises a set of the three CDR amino acid sequences that are in the T0235005G01 or T023500043 sdAbs described in Table B of US 20190367598A1. In an embodiment, AB consists essentially of, or consists of, the amino acid sequence of T0235005G01 or T023500043 described in Table B of US 20190367598A1. In an embodiment, a BBB-transporting fusion protein of the present disclosure cross-competes for binding to HSA with the T0235005G01 or T023500043 sdAbs described in Table B of US 20190367598A1.

In an embodiment, the AB domain comprises a set of the three CDR amino acid sequences that are in the R28, R11, M75 or M79 sdAbs described in WO 2019/204925. These CDR sequences are set forth in Table 2A below.

TABLE 2A
Exemplary CDR amino acid sequences for
the AB domain.

| sdAb | CDR1 | CDR2 | CDR3 |
|---|---|---|---|
| R11 | GFLLRSNTM | IRPSGLT | HTRPPFQRDS |
| | (SEQ ID | (SEQ ID | (SEQ ID NO: 43) |
| | NO: 41) | NO: 42) | or |
| | | | ATRPPFQRDS |
| | | | (SEQ ID NO: 44) |
| R28 | GRTFIAYA | ITNFAGGTT | AADRSAQTMRQVRPVLPY |
| | (SEQ ID | (SEQ ID | (SEQ ID NO: 3) |
| | NO: 1) | NO: 2) | |
| M75 | GRTFDNYVM | ISGSGSIT | AAGSRRTYYREPKFYPS |
| | (SEQ ID | (SEQ ID | (SEQ ID NO: 47) |
| | NO: 45) | NO: 46) | |
| M79 | GSTESSSSV | ITSGGST | NVAGRNWVPISRYSPGPY |
| | (SEQ ID | (SEQ ID | (SEQ ID NO: 50) |
| | NO: 48) | NO: 49) | or |
| | | | AVAGRNWVPISRYSPGPY |
| | | | (SEQ ID NO: 51) |

In an embodiment, AR consists essentially of, or consists of, the amino acid sequence of the R28, R11, M75 or M79 sdAbs described in WO 2019/204925. In an embodiment, AR consists essentially of, or consists of, the amino acid sequence of one of the humanized variants of R28, R11, M75 or M79 described in WO 2019/204925. The amino acid sequences of the parental and humanized variants of R28, R11, M75 and M79 are shown in the SEQUENCES Table on pages 46-48 of WO 2019/204925. In an embodiment, AR consists essentially of, or consists of, an amino acid sequence selected from the parental and humanized sequences shown in Table 2B herein below. In an embodiment, AR consists essentially of, or consists of, the parental or humanized amino acid sequence of R28 shown in Table 2B below.

TABLE 2B
Exemplary amino acid sequences for the AB domain.

| sdAb | Parental Sequence | Exemplary Humanized Sequence |
|---|---|---|
| R11 | QVKLEESGGGLVQAGGSLRLSCVGPGEL | QVQLVESGGGLVQPGGSLRLSCAGPGFL |
| | LRSNTMGWYRQAPGKERELVAFIRPSGL | LRSNTMGWYRQAPGKERELVAFIRPSGL |
| | TNYNDAVQGRFTISRDNAKNTVYLQMNA | TNYNDAVQGRFTISRDNAKNTVYLQMNS |
| | LKPEDTAVYYCHTRPPFQRDSWGQGTQV | LRAEDTAVYYCHTRPPFQRDSWGQGTLV |
| | TVSS (SEQ ID NO: 52) | TVSS (SEQ ID NO: 53) |
| R28 | QVQLVESGGGLVQAGGSLRLSCVASGRT | QVQLVESGGGLVQPGGSLRLSCAASGRT |
| | FIAYAMGWFRQAPGKEREFVAAITNFAG | FIAYAMGWFRQAPGKEREFVAAITNFAG |
| | GTTYYADSVKGRFTISRDNAKTTVYLQM | GTTYYADSVKGRFTISRDNAKTTVYLQM |
| | NSLKPEDTALYYCAADRSAQTMRQVRPV | NSLRAEDTAVYYCAADRSAQTMRQVRPV |
| | LPYWGQGTQVTVSS (SEQ ID NO: | LPYWGQGTLVTVSS (SEQ ID NO: |
| | 4) | 5) |
| M75 | QVQLVESGGGFVQAGGSLRLSCAASGRT | EVQLVESGGGLVQPGGSLRLSCAASGRT |
| | FDNYVMAWFRQAPGKEREFVASISGSGS | FDNYVMAWFRQAPGKGLEFVSSISGSGS |
| | ITNYANSVKDRFTISRDSAKNAIYLQMN | ITNYANSVKDRFTISRDNSKNTLYLQMN |
| | SLKPEDTALYYCAAGSRRTYYREPKFYP | SLRAEDTAVYYCAAGSRRTYYREPKFYP |
| | SWGQGTQVTVSS (SEQ ID NO: 54) | SWGQGTLVTVSS (SEQ ID NO: 55) |
| M79 | QVKLEESGGGLVQAGGSLKLSCAASGST | EVQLVESGGGLVQPGGSLRLSCAASGST |
| | FSSSSVGWYRQAPGQQRELVAAITSGGS | FSSSSVGWYRQAPGKGLELVSAITSGGS |
| | TNTADSVKGRFTMSRDNAKNTVYLQMRD | TNTADSVKGRFTISRDNSKNTLYLQMNS |
| | LKPEDTAVYYCNVAGRNWVPISRYSPGP | LRAEDTAVYYCNVAGRNWVPISRYSPGP |
| | YWGQGTQVTVSS (SEQ ID NO: 56) | YWGQGTLVTVSS (SEQ ID NO: 57) |

### IGF1R Binding Domain RB

The RB domain of the BBB-transporting fusion proteins of the disclosure confers upon the fusion protein the capability of crossing the BBB via transcytosis mediated by the IGF1R, also known as CD221, IGF1R, IGFR, and JTK13. Human IGF1R is synthesized as a monomeric 1367-amino acid pre-proreceptor with a 30-amino acid signal sequence (UniProtKB—P08069). Following cleavage of the signal peptide, the proreceptor is glycosylated, dimerized, and transported to the Golgi apparatus, where furin cleavage yields alpha and beta subunits that form a disulfide-linked tetramer (beta-alpha-alpha-beta), which is transported to the plasma membrane. The fully mature cell membrane-bound IGF1R consists of two 130- to 135-kDa alpha subunits and two 90- to 95-kDa beta subunits, with several alpha-alpha and alpha-beta disulfide bonds. The alpha subunits are entirely extracellular and form the ligand-binding domain. The beta subunits contain an extracellular domain, a transmembrane domain and an intracellular domain. Ligand binding induces transautophosphorylation and phosphorylation of a wide variety of downstream signaling molecules.

The ability of the RB domain to confer hIGF1R mediated transcytosis of a fusion protein described herein may be assessed by any method known in the art. For example, in vitro assays may evaluate internalization of a fusion protein into IGF1-R expressing cells such as MCF-7 cells or primary human microvascular brain endothelial cells (HMBEC) as described in [0317]-[0315] of EP3725806A1. A similar assay that may be used when RB cross-reacts with rat IGF1-R employs immortalized rat brain endothelial cells (svARBEC) as described in U.S. Pat. No. 10,100,117. Also, known in vitro and in vivo BBB models may be used to evaluate BBB-transporting ability of fusion proteins that bind to human and rat IGF1R, e.g., as described in Examples 10 and 12 of U.S. Pat. No. 10,100,117.

In some embodiments, the RB domain is specific for IGF1R, i.e., it does not substantially bind to the insulin receptor or any other non-IGF1R proteins. The fusion protein containing RB should not induce signaling through IGF1R or IR, and also should not inhibit signaling through IGF1R or IR that is induced by insulin, IGF-1 or IGF-2. The signaling impact of a BBB-transporting fusion protein described herein can be assessed by analyzing phosphorylation of the IGF1R and IR and/or of the receptor-stimulated downstream kinase Akt. This assessment may be performed using any method known in the art, e.g., as described in Example 14 of U.S. Pat. No. 10,100,117 or in paragraphs [0325]-[0327] of EP3725806A1. Any impact of the fusion protein on IGF1 signaling through the IGF1R may be assessed using any method known in the art, e.g., by an MCF-7 cell line proliferation assay as described in [0320]-[0324] of EP3725806A1.

The potential for interference with ligand binding can be reduced by deriving the RB domain from an anti-hIGF1R antibody molecule that is known not to interfere with ligand binding or selecting an antibody that does not binds to the alpha subunit. In an embodiment, the fusion protein cross-competes via the RB domain for hIGF1R binding with the IGF1R-5 sdAb described in U.S. Pat. No. 10,100,117, the IGF1R-3 sdAb described in U.S. Pat. No. 10,106,614 or the IGF1R-5 sdAb described in U.S. Pat. No. 10,112,998. In an embodiment, the fusion protein cross-competes via the RB domain for hIGF1R binding with any of the 996, 1226 and 1564 antibodies described in EP3725806A1. In an embodiment, the epitope for the RB domain has three binding sites: binding site 1 includes one or more of R650, Y775, P776, F778, E779, S791, and L798; binding site 2 includes one or more of L641, H808, E809 and L813; and binding site 3 includes one or more of V397, W434, D435, Y460 and C488.

In some embodiments, RB (as part of the fusion protein) also binds to (e.g., cross-reacts with) IGF1R from one, two, three, four or more other mammalian species, e.g., any combination of two, three, four or more of mouse, rat, guinea pig, hamster, rabbit, cat, dog, pig, sheep, horse, cow and monkey (e.g., rhesus and/or cynomolgus). In an embodiment, the fusion protein binds via the RB domain to IGF1R from at least mouse, rat, monkey (rhesus or cynomolgus) and human. In an embodiment, the fusion protein binds via the RB domain to IGF1R from at least mouse, dog and human.

In some embodiments, the RB domain (as part of the fusion protein) binds to hIGF1R, and optionally to at least one other mammalian IGF IR, with a desired affinity within a pH range of between about 5.0 or about 5.5 up to about 7.4. In an embodiment, the desired affinity is a dissociation constant (K_{D}) ranging from: (i) about 0.1 nM to about 1,000 nM; (ii) about 0.2 nM to about any one of 500 nM, 250 nM, 100 nM, 50 nM, 25 nM or 10 nM; (iii) about 0.5 nM to about any one of 250 nM, 100 nM, 50 nM, 25 nM, 10 nM or 5 nM; or (iv) about 1 nM to about any one of 100 nM, 50 nM, 25 nM, 10 nM or 5 nM. In an embodiment, the desired affinity is a dissociation constant (K_{D}) of 1 nM to 10 nM. In an embodiment, the affinity is determined by surface plasmon resonance (SPR) at 25° C. and a pH range of about 5.5 to about 7.4. In an embodiment, the affinity of the fusion protein for IGF1R from mouse, rat and monkey is similar to the affinity for hIGF1R, e.g., a K_{D} within 70% to 130%, 80% to 120%, or 90% to 110% of the K_{D} for hIGF1R. An exemplary SPR assay for measuring K_{D} of a BBB-containing fusion protein is described in U.S. Pat. No. 10,100,117.

The RB domain of a BBB-transporting fusion protein of the disclosure comprises the amino acid sequence of a heavy chain variable region (HCVR) of an anti-hIGF1R antibody. To facilitate expression of the fusion protein by genetically-modified cells, in some embodiments, the RB domain has a molecular weight of less than about any of 75 kDa, 50 kDa or 25 kDa. The RB domain may be derived from any anti-IGF1R antibody molecule known in the art, including conventional 4-chain antibodies, antigen-binding fragments, Fab, Fab′, F(ab′)₂, Fv (double-chain and single-chain (scFv)), minibody, diabody and sdAb. In an embodiment, RB consists essentially of, or consists of, the HCVR amino acid sequence of a sdAb.

In an embodiment, the RB domain comprises a set of the three CDR amino acid sequences in a sdAb selected from the group consisting of: the IGF1R-5 sdAb described in U.S. Pat. No. 10,100,117, the IGF1R-3 sdAb described in U.S. Pat. No. 10,106,614 and the IGF1R-5 sdAb described in U.S. Pat. No. 10,112,998. These CDR sequences are set forth in Table 3A below.

TABLE 3A
Exemplary CDR amino acid sequences
for the RB Domain

| sdAb | CDR1 | CDR2 | CDR3 |
|---|---|---|---|
| IGF1R-5 | GRTIDNYA | IDWGDGGX, | AMARQSRVNLDVARYDY |
| | (SEQ ID | where X is | (SEQ ID NO: 9) |
| | NO: 7) | A or T (SEQ | |
| | | ID NO: 8) | |
| IGF1R-3 | EYPSNFYA | VSRDGLTT | AIVITGVWNKVDVNSRS |
| | (SEQ ID | (SEQ ID NO: | YHY (SEQ ID NO: |
| | NO: 58) | 59) | 60) |
| IGF1R-4 | GGTVSPTA | ITWSRGTT | AASTFLRILPEESAYTY |
| | (SEQ ID | (SEQ ID NO: | (SEQ ID NO: 63) |
| | NO: 61) | 62) | |

In an embodiment, RB consists essentially of, or consists of, the parental amino acid sequence of IGF1R-5, IGF1R-3 or IGF1R-4, or a humanized variant of one of these sdAbs. In an embodiment, RB consists essentially of, or consists of a sequence selected from the group consisting of:
(i) X₁VX₂LX₃ESGGGLVQX₄GGSLRLSCAASGRTIDNYAMAWX₅RQAPGKX₆X₇EX₈V X₉TIDWGDGGX₁₀RYANSVKGRFTISRDNX₁₁KX₁₂TX₁₃YLQMNX₁₄LX₁₅X₁₆EDTAV YX₁₇CAMAR QSRVNLDVARYDYWGQGTX₁₈VTVSS, where X₁ is E or Q; X₂ is K or Q; X₃ is V or E; X₄ is A or P; X₅ is V or S; X₆ is D or G; X₇ is L or R; X₅ is F or W; X₉ is A or S; X₁₀ is A or T; X₁₁ is A or S; X₂ is G or N; X₁₃ is M or L; X₁₄ is N or R; X₁₅ is E or R; X₁₆ is P or A; X₁₇ is S or Y; and X₁₈ is Q or L (SEQ ID NO:64);
(ii) X₁VX₂LX₃ESGGGLVQX₄GGSLRLSCX₅ASEYPSNFYAMSWX₆RQAPGKX₇X₈EX₉V X₁₀GVSRDGLTTLYADSVKGRFTX₁₁SRDNX₁₂KNTX₁₃X₁₄LQMNSX₁₅X₁₆AEDTAVY YCAIVITG VWNKVDVNSRSYHYWGQGTX₁₇VTVSS, where X₁ is E or Q; X₂ is K or Q; X₃ is V or E; X₄ is A or P; X₅ is V or A; X₆ is F or V; X₇ is E or G; X₈ is R or L; X₉ is F or W; X₁₀ is A or S; X₁₁ is M or; X₁₂ is A or S; X₁₃ is V or L; X₁₄ is D or Y; X₁₅ is V or L; X₁₆ is K or R; and X₁₇ is Q or L (SEQ ID NO:65); and
(iii) X₁VX₂LX₃ESGGGLVQX₄GGSLRLSCX₅X₆SGGTVSPTAMGWX₇RQAPGKX₈X₉EX₁₀ VX₁₁HITWSRGTTRX₁₂ASSVKX₁₃RFTISRDX₁₄X₁₅KNTX₁₆YLQMNSLX₁₇X₁₈EDTA VYYCAAS TFLRILPEESAYTYWGQGT X₁₉VTVSS, where X₁ is E or Q; X₂ is K or Q; X₃ is V or E; X₄ is A or P; X₅ is A or E; X₆ is V or A; X₇ is V or F; X₈ is G or E; X₃ is L or R; X₁₀ is F or W; X₁₁ is G or S; X₁₂ is V or Y; X₁₃ is D or G; X₁₄ is N or S; X₁₅ is A or S; X₁₆ is L or V; X₁₇ is K or R; X₁₈ is A or S; and X₁₉ is L or Q (SEQ ID NO:66).

In an embodiment, RB consists essentially of, or consists of, the amino acid sequence of any of the humanized variants of IGF1R-5, IGF1R-3 or IGF1R-5 described in U.S. Pat. Nos. 10,100,117, 10,106,614 and 10,112,998, respectively. In an embodiment, RB consists essentially of, or consists of, an amino acid sequence selected from the parental or humanized sequences shown in Table 3B herein below. In an embodiment, RB consists essentially of, or consists of, the parental or humanized amino acid sequence of IGF1R-5 shown in Table 3B below.

TABLE 3B
Exemplary amino acid sequences for the RB domain.

| sdAb | Parental Sequence | Exemplary Humanized Sequence |
|---|---|---|
| IGF1R-5 | QVKLEESGGGLVQAGGSLRLSCAASGRT | QVQLVESGGGLVQPGGSLRLSCAASGRT |
| | IDNYAMAWSRQAPGKDREFVATIDWGDG | IDNYAMAWVRQAPGKGLEWVATIDWGDG |
| | GARYANSVKGRFTISRDNAKGTMYLQMN | GTRYANSVKGRFTISRDNSKNTMYLQMN |
| | NLEPEDTAVYSCAMARQSRVNLDVARYD | SLRAEDTAVYYCAMARQSRVNLDVARYD |
| | YWGQGTQVTVSS (SEQ ID NO: 11) | WGQGTLVTVSS (SEQ ID NO: 12) |
| IGF1R-3 | QVKLEESGGGLVQAGGSLRLSCVASEYP | EVQLVESGGGLVQPGGSLRLSCAASEYP |
| | SNFYAMSWFRQAPGKEREFVAGVSRDGL | SNFYAMSWFRQAPGKEREFVSGVSRDGL |
| | TTLYADSVKGRFTMSRDNAKNTVDLQMN | TTLYADSVKGRFTISRDNSKNTLYLOMN |
| | SVKAEDTAVYYCAIVITGVWNKVDVNSR | SLRAEDTAVYYCAIVITGVWNKVDVNSR |
| | SYHYWGQGTQVTVSS (SEQ ID NO: | SYHYWGQGTLVTVSS (SEQ ID NO: |
| | 67) | 68) |
| IGF1R-4 | QVKLEESGGGLVQAGGSLRLSCEVSGGT | QVQLVESGGGLVQPGGSLRLSCAVSGGT |
| | VSPTAMGWFRQAPGKEREFVGHITWSRG | VSPTAMGWVRQAPGKGLEWVGHITWSRG |
| | TTRVASSVKDRFTISRDSAKNTVYLQMN | TTRYASSVKGRFTISRDNSKNTVYLOMN |
| | SLKSEDTAVYYCAASTFLRILPEESAYT | SLRAEDTAVYYCAASTFLRILPEESAYT |
| | YWGQGTQVTVSS (SEQ ID NO: 69) | YWGQGTLVTVSS (SEQ ID NO: 70) |

Generation of HSA and hIGF1R Binding Domains

The AB and RB domains may be derived from any antibody or antigen binding fragment thereof that has the desired properties described herein. The antibody or antigen binding fragment may be already known in the art or identified by via any approach known in the art.

In some embodiments, one or both of the AB and RB domains are derived from a single-domain Ab. For example, sdAbs of camelid origin lack light chains and thus their antigen binding sites consist of one domain, termed V_{H}H. sdAb have also been observed in shark and are termed V_{NAR}. Other sdAb may be engineered based on human Ig heavy and light chain sequences. As used herein, the term “sdAb” includes sdAbs directly isolated from V_{H}, V_{H}H, V_{L}, or V_{NAR} reservoir of any origin through phage display or other technologies, recombinantly produced sdAbs, as well as those sdAb generated through further modification of such sdAbs by humanization, affinity maturation, stabilization, solubilization, or other methods of antibody engineering. Also encompassed by the present disclosure are homologues, derivatives, or fragments that retain the antigen-binding function and specificity of the parental sdAb.

To generate V_{H}H sdAbs that bind to HSA and V_{H}H sdAbs that bind to hIGF1R, the skilled person may employ any approach known in the art, e.g., substantially as described in WO 2019/204925 and U.S. Pat. No. 10,100,117, respectively, to generate and screen a phage-displayed V_{H}H library from the heavy-chain-only antibody repertoire of llama or other camelid immunized with a desired antigen. In an embodiment, the IGF1R antigen used to immunize the camelid is a fragment of precursor hIGF1R, e.g., comprises amino acids 1 to 932 of the sequence shown in FIG. 1. In an embodiment, the antigenic hIGF1R fragment does not contain the signal peptide.

AB or RB domains derived from sdAbs may include the parental framework regions; alternatively, the parental CDRs may be grafted onto V_{NAR}, V_{H}H, V_{H} or V_{L} framework regions of other sdAbs or onto the framework regions of other types of antibody fragments or antibody-like molecules (Fv, scFv, Fab) of any source (e.g., human) or proteins of similar size and nature onto which CDRs can be grafted (for example, see Nicaise, M. et al, Protein Sci 13:1882-91 2004).

In some embodiments, the amino acid sequence in one or both of the AB and RB domains is a humanized version (humanized variant) of the parental variable region. Humanization of an antibody or antibody fragment comprises replacing an amino acid in the sequence with its human counterpart, as found in the human consensus sequence, without loss of antigen-binding ability or specificity; this approach reduces immunogenicity of the antibody or fragment thereof when introduced into human subjects. The parental sequence may be humanized using any suitable method known in the art, for example, but not limited to CDR grafting and veneering.

In the process of CDR grafting, one or more than one of the CDR defined herein may be fused or grafted to a human variable region (V_{H}, or V_{L}), to another human antibody (IgA, IgD, IgE, IgG, and IgM), to antibody fragment framework regions (Fv, scFv, Fab), or to proteins of similar size and nature onto which CDR can be grafted. CDR grafting is known in the art and is described in at least the following: U.S. Pat. Nos. 6,180,370, 5,693,761, 6,054,297, and European Patent No. 626390.

Veneering, also referred to in the art as “variable region resurfacing”, involves humanizing solvent-exposed positions of the antibody or antibody fragment; thus, buried non-humanized residues, which may be important for CDR conformation, are preserved while the potential for immunological reaction against solvent-exposed regions is minimized. Veneering is known in the art and is described in at least the following: U.S. Pat. Nos. 5,869,619, 5,766,886, and 5,821,123, and European Patent No. 519596.

### Linkers

The BBB-transporting fusion protein may contain one or more linkers, e.g., between the AB and RB domains and/or between the RB domain and the cargo moiety (e.g., therapeutic polypeptide). Each linker should be of sufficient length to allow the linked polypeptides to individually fold into 3-dimensional structures having the desired functional activity, e.g., binding or therapeutic. Also, each linker should not be cleavable by any proteases or other enzymes present in the serum.

In some embodiments, the linker is a peptide linker comprising at least two, three or four amino acids and less than about 30 amino acids, e.g., less than about any of 25, 20, 15 or 10 amino acids. Peptide linkers are known in the art and nonlimiting examples are described herein.

The peptide linker may have a naturally occurring sequence, or a non-naturally occurring sequence. For example, a sequence derived from the hinge region of heavy chain only antibodies may be used as the linker. See, for example, WO1996/34103.

Suitable linker peptides typically include G and/or S residues in various formats, with exemplary linkers including GGGG (SEQ ID NO:71), TGGGG (SEQ ID NO:72), GGSSGGSGSSSGSGGSGSSG (SEQ ID NO:73), (GGSS)n (SEQ ID NO:74), (GGGGS)n (SEQ ID NO:13), (SGGGG)n (SEQ ID NO:75) and GGGG(SGGGG)n (SEQ ID NO:76), wherein “n” in each case is generally a number between 1 and 10, e.g., 1, 2, 3, 4, 5, 6, 7, 8, 9 or 10, provided that the maximum length of about 40 amino acids is not exceeded. Another exemplary peptide linker is SKPTCPPPELLGGPSVFIFPPK (SEQ ID NO:77).

In an embodiment, the fusion protein comprises two peptide linkers L1 and L2, with peptide linker L1 located between AB and RB and L2 located between RB and the cargo polypeptide. In an embodiment, the length of each of L1 and L2 is between about 15 and 30 amino acids, or between about 20 and 25 amino acids. L1 and L2 may have the same or different amino acid sequences. In an embodiment, each L1 and L2 independently consists essentially of, or consists of: (GGGGS)₄ (SEQ ID NO:14) or (GGGGS)s (SEQ ID NO:78). In an embodiment, each of L1 and L2 consists essentially of, or consists of: (GGGGS)₄.

### Therapeutic and Diagnostic Cargos

The cargo moiety may be any therapeutic or diagnostic molecule that may be joined or linked to the hIGF1R binding domain.

In an embodiment, the therapeutic molecule is a polypeptide, e.g., a cytokine, an enzyme, a growth factor, or an antibody or antigen binding fragment thereof. In an embodiment, the polypeptide has activity useful for treating a neurological disorder in a mammal, e.g., a human. In an embodiment, the neurological disorder is selected from the group consisting of: LSDs with neurological manifestations (nLSDs), Alzheimer's disease (AD), ataxias (e.g., hereditary ataxias such as Friedreich's ataxia), Huntington's disease, stroke, dementia, muscular dystrophy (MD), multiple sclerosis (MS), amyotrophic lateral sclerosis (ALS), cystic fibrosis, Angelman's syndrome Liddle syndrome, Parkinson's disease, Pick's disease, Paget's disease, schizophrenia, depression, encephalitis, prion diseases, cancer and traumatic brain injury.

In an embodiment, the therapeutic molecule is an enzyme. In an embodiment, the enzyme is deficient in an LSD, e.g., any of the deficient enzymes listed in Table 1 of Edelman, M. J. and Maegawa, G. H. B., Frontiers in Molecular Biosciences, Volume 7, Article 559804 (12 Nov. 2020). In an embodiment, the enzyme is IDUA, IDS, SGSH, GLA or GAA. In an embodiment, the LSD is MPS-1 and the enzyme is IDUA. In an embodiment, the enzyme is IDS. In an embodiment, the enzyme is not IDS.

In an embodiment, the therapeutic molecule is an antibody or antigen-binding fragment thereof that specifically binds to a target protein (e.g., antigen) in the brain. In an embodiment, the target protein is a Tau protein (e.g., cis P-tau), Abeta, BACE1 or a splice isoform thereof, human epidermal growth factor receptor (2)(HER2), apolipoprotein E4 (ApoE4), alpha-synuclein, CD20, prion protein (PrP), leucine rich repeat kinase 2 (LRRK2), parkin presenilin 1, presenilin 2, gamma secretase, death receptor 6 (DR6), amyloid precursor protein (APP), p75 neurotrophin receptor (p75NTR), interferon gamma, interleukin-1 receptor (IL-1R), interleukin-6 (IL-6), interleukin 6 receptor (IL6R), interleukin-12, interleukin-23, TNF receptor (TNFR1), interleukin 1 beta (IL1beta), caspase 6, interleukin-17 (IL-17), CTLA-4, programmed death receptor 1 (PD-1), programmed death-ligand 1 (PD-L1), programmed death-ligand 2 (PD-L2), TNF-alpha, vascular endothelial growth factor (VEGF), or VEGF receptor (VEGFR).

In an embodiment, the therapeutic molecule is a cytokine or growth factor, e.g., an immunomodulatory cytokine, e.g., granulocyte-macrophage colony-stimulating factor (GM-CSF), interferon-beta, nerve growth factor, glial-cell line-derived neurotrophic factor (GNDF), ciliary neurotrophic factor (CNTF), basic fibroblast growth factor (bFGF), fibroblast growth factor-2 (FGF2), epidermal growth factor (EGF), or transforming growth factor (TGF)-beta 2.

### Genetically-Modified Cells

Any of the BBB-transporting fusion proteins described above may be expressed by a mammalian cell(s) genetically modified to express and secrete the fusion protein. The genetically modified cell(s) may be derived from a variety of different mammalian cell types (e.g., human cells), including epithelial cells, endothelial cells, fibroblast cells, mesenchymal stem cells, keratinocyte cells and stem cells, e.g., embryonic stem cells or induced pluripotent stem cells. Exemplary cell types include the cell types recited in WO 2017/075631. In some embodiments, the cells are derived from a cell-line shown in Table 4 below.

TABLE 4
Exemplary cell lines

| | | Germ | |
|---|---|---|---|
| Cell Line | Cell Type | Layer | Commercial Source |
| ARPE-19 | Epithelial (Retinal) | Ectoderm | ATCC (CRL-2302) |
| BJ | Fibroblast (Foreskin) | Ectoderm | ATCC (CRL-2522) |
| CCD-841-CoN | Epithelial (Colon) | Endoderm | ATCC (CRL-1790) |
| HaCat | Keratinocyte | Ectoderm | Addexbio (T0020001) |
| HHSEC | Endothelial (Hepatic Sinusoidal) | Endoderm | Sciencellonline.com (#5000) |
| Huv-EC-C | Endothelial (Embryonic umbilical) | Mesoderm | ATCC (CRL-1730) |
| MCF-10A | Epithelial (Mammary Gland) | Ectoderm | ATCC (CRL-10317) |
| MRC-5 | Fibroblast (Lung) | Mesoderm | ATCC (CCL-171) |
| MSC, human | Mesenchyme (Bone Marrow) | Mesoderm | ATCC (PCS-500-012) |
| MSC, mouse | Mesenchyme (Bone Marrow) | Mesoderm | Cyagen (MU BMX-01001) |
| WS-1 | Fibroblast (Skin) | Ectoderm | ATCC (CRL-1502) |
| 293F | Epithelial (Embryonic Kidney) | Mesoderm | Thermo Fisher (R790007) |

In an embodiment, any of the genetically modified mammalian cells described herein is derived from an RPE cell, e.g., an ARPE-19 cell. In an embodiment, a genetically modified ARPE-19 cell comprises any of the expression cassettes, transposons and polynucleotides described herein.

Cells may be genetically modified to express and secrete a desired BBB-transporting fusion protein using any of a variety of genetic engineering techniques known in the art. For example, a cell may be transfected with an expression vector comprising an exogenous nucleotide sequence(s) encoding the desired fusion protein operably linked to control elements necessary or useful for gene expression, e.g., promoters, ribosomal binding sites, enhancers, polyA signal and the like. In some embodiments, the exogenous nucleotide sequence is part of a transcription unit that is stably integrated into the genome of the parental cell.

In an embodiment, the exogenous sequence includes a nucleotide sequence encoding a secretory signal sequence for the fusion protein. In an embodiment, the signal sequence is from a naturally secreted protein. In an embodiment, the signal sequence is MELGLSWVVLAALLQGVQA (SEQ ID NO:79). In some embodiments, the signal sequence consists essentially of an amino acid sequence shown in Table 5 below.

TABLE 5
Exemplary secretory signal peptide sequences

| Protein | Amino Acid Sequence |
|---|---|
| Albumin | MKWVTFISLLFLFSSAYS (SEQ ID |
| | NO: 80) |
| Kappa Leader | MVLQTQVFISLLLWISGAYG (SEQ |
| | ID NO: 81) |
| Plasminogen | MQMSPALTCLVLGLALVFGEGSA |
| activator | (SEQ ID NO: 82) |
| inhibitor 1 | |
| Thrombospondin-1 | MGLAWGLGVLFLMHVCGT (SEQ ID |
| | NO: 83) |
| Fibronectin | MLRGPGPGLLLLAVQCLGTAVPSTGA |
| | SKSKR (SEQ ID NO: 84) |
| Basement membrane- | MGWRAAGALLLALLLHGRLLA (SEQ |
| specific heparan | ID NO: 85) |
| sulfate | |
| proteoglycan | |
| core protein | |
| (HSPG2) | |
| Agrin | MAGRSHPGPLRPLLPLLVVAACVLPG |
| | AGG (SEQ ID NO: 86) |

The genetically modified mammalian cells for use in devices, compositions and methods described herein, e.g., as a plurality of cells in a hydrogel capsule, may be in various stages of the cell cycle. In some embodiments, at least one cell in the plurality of genetically modified cells is undergoing cell division. Cell division may be measured using any known method in the art, e.g., as described in DeFazio A et al (1987) J Histochem Cytochem 35:571-577 and Dolbeare F et al (1983) Proc Natl Acad Sci USA 80:5573-5577, each of which is incorporated by reference in its entirety. In an embodiment at least 1, 2, 3, 4, 5, 10, or 20% of the cells are undergoing cell division, e.g., as determined by 5-ethynyl-2′deoxyuridine (EdU) assay or 5-bromo-2′-deoxyuridine (BrdU) assay. In some embodiments, cell proliferation is visualized or quantified by microscopy (e.g., fluorescence microscopy (e.g., time-lapse or evaluation of spindle formation) or flow cytometry. In some embodiments, none of the cells in the plurality of genetically modified cells are undergoing cell division and are quiescent. In an embodiment, less than 1, 2, 3, 4, 5, 10, or 20% of the cells are undergoing cell division, 5-ethynyl-2′deoxyuridine (EdU) assay, 5-bromo-2′-deoxyuridine (BrdU) assay, microscopy (e.g., fluorescence microscopy (e.g., time-lapse or evaluation of spindle formation), or flow cytometry.

In an embodiment, at least 50%, 60%, 70%, 80%, 90% or more of the genetically modified cells in the plurality are viable. Cell viability may be measured using any known method in the art, e.g., as described in Riss, T. et al (2013) “Cell Viability Assays” in Assay Guidance Manual (Sittapalam, G. S. et al, eds). For example, cell viability may be measured or quantified by an ATP assay, 5-ethynyl-2′deoxyuridine (EdU) assay, 5-bromo-2′-deoxyuridine (BrdU) assay. In some embodiments, cell viability is visualized or quantified by microscopy (e.g., fluorescence microscopy (e.g., time-lapse or evaluation of spindle formation) or flow cytometry. In an embodiment, at least 80% of the engineered cells in the plurality are viable, e.g., as determined by an ATP assay, a 5-ethynyl-2′deoxyuridine (EdU) assay, a 5-bromo-2′-deoxyuridine (BrdU) assay, microscopy (e.g., fluorescence microscopy (e.g., time-lapse or evaluation of spindle formation), or flow cytometry.

Any of the parameters described herein may be assessed using standard techniques known to one of skill in the art, such as histology, microscopy, and various functional assays.

### Implantable Cell-Encapsulating Devices

A genetically modified cell described herein or a plurality of such cells may be incorporated into an implantable device for use in providing a therapeutic or diagnostic cargo to a subject with a CNS disease or condition, e.g., to a patient with an LSD, e.g., MPS-I.

An implantable device of the present disclosure comprises at least one barrier that prevents immune cells from contacting cells contained inside the device. At least a portion of the barrier needs to be sufficiently porous to allow proteins (e.g., the fusion protein) expressed and secreted by the cells to exit the device. A variety of device configurations known in the art are suitable.

The device (e.g., particle) can have any configuration and shape appropriate for supporting the viability and productivity of the contained cells after implant into the intended target location. As non-limiting examples, device shapes may be cylinders, rectangles, disks, ovoids, stellates, or spherical. The device can be comprised of a mesh-like or nested structure. In some embodiments, a device is capable of preventing materials over a certain size from passing through a pore or opening. In some embodiments, a device (e.g., particle) is capable of preventing materials greater than 50 kD, 75 kD, 100 kD, 125 kD, 150 kD, 175 kD, 200 kD, 250 kD, 300 kD, 400 kD, 500 kD, 750 kD, or 1,000 kD from passing through.

In an embodiment, the device is a macroencapsulation device. Nonlimiting examples of macrodevices are described in: WO 2019/068059, WO 2019/169089, U.S. Pat. Nos. 9,526,880, 9,724,430 and 8,278,106; European Patent No. EP742818B1, and Sang, S. and Roy, S., Biotechnol. Bioeng. 113(7):1381-1402 (2016).

In an embodiment, the device is a macrodevice having one or more cell-containing compartments. A device with two or more cell-containing compartments may be configured to produce two or more proteins, e.g., cells expressing the fusion protein would be placed in one compartment and cells expressing a different protein (e.g., a therapeutic protein that can alleviate one or more symptoms of the targeted CNS disease or condition) would be placed in a separate compartment. WO 2018/232027 describes a device with multiple cell-containing compartments formed in a micro-fabricated body and covered by a porous membrane.

In an embodiment, the device is configured as a thin, flexible strand as described in U.S. Pat. No. 10,493,107. This strand comprises a substrate, an inner polymeric coating surrounding the substrate and an outer hydrogel coating surrounding the inner polymeric coating. The protein-expressing cells are positioned in the outer coating.

In some embodiments, a device (e.g., particle) has a largest linear dimension (LLD), e.g., mean diameter, or size that is at least about 0.5 millimeter (mm), preferably about 1.0 mm, about 1.5 mm or greater. In some embodiments, a device can be as large as 10 mm in diameter or size. For example, a device or particle described herein is in a size range of 0.5 mm to 10 mm, 1 mm to 10 mm, 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm.

In some embodiments, a device of the disclosure (e.g., particle, capsule) comprises at least one pore or opening, e.g., to allow for the free flow of materials. In some embodiments, the mean pore size of a device is between about 0.1 μm to about 10 μm. For example, the mean pore size may be between 0.1 μm to 10 μm, 0.1 μm to 5 μm, 0.1 μm to 2 μm, 0.15 μm to 10 μm, 0.15 μm to 5 μm, 0.15 μm to 2 μm, 0.2 μm to 10 μm, 0.2 μm to 5 μm, 0.25 μm to 10 μm, 0.25 μm to 5 μm, 0.5 μm to 10 μm, 0.75 μm to 10 μm, 1 μm to 10 μm, 1 μm to 5 μm, 1 μm to 2 μm, 2 μm to 10 μm, 2 μm to 5 μm, or 5 μm to 10 μm. In some embodiments, the mean pore size of a device is between about 0.1 μm to 10 μm. In some embodiments, the mean pore size of a device is between about 0.1 μm to 5 μm. In some embodiments, the mean pore size of a device is between about 0.1 μm to 1 μm.

In some embodiments, the device comprises a semi-permeable, biocompatible membrane surrounding the genetically modified cells that are encapsulated in a polymer composition (e.g., an alginate hydrogel). The membrane pore size is selected to allow oxygen and other molecules important to cell survival and function to move through the semi-permeable membrane while preventing immune cells from traversing through the pores. In an embodiment, the semi-permeable membrane has a molecular weight cutoff of less than 1000 kD or between 50-700 kD, 70-300 kD, or between 70-150 kD, or between 70 and 130 kD.

In an embodiment, the device may contain a cell-containing compartment that is surrounded with a barrier compartment formed from a cell-free biocompatible material, such as the core-shell microcapsules described in Ma, M et al., Adv. Healthc Mater., 2(5):667-672 (2012). Such a barrier compartment could be used with or without the semi-permeable membrane.

Cells in the cell-containing compartment(s) of a device of the disclosure may be encapsulated in a polymer composition. The polymer composition may comprise one or more hydrogel-forming polymers. In addition to the polymer composition in the cell-containing compartment(s), the device (e.g., macrodevice, particle, hydrogel capsule) may comprise or be formed from materials such as metals, metallic alloys, ceramics, polymers, fibers, inert materials, and combinations thereof. A device may be completely made up of one type of material, or may comprise other materials within the cell-containing compartment and any other compartments.

In some embodiments, the device comprises a metal or a metallic alloy. In an embodiment, one or more of the compartments in the device (e.g., the first compartment, the second compartment, or all compartments) comprises a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys, e.g., ELGILOY® and PHYNOX®). For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S). In metal-containing devices, the amount of metal (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device comprises a ceramic. In an embodiment, one or more of the compartments in the device (e.g., the first compartment, the second compartment, or all compartments) comprises a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used. In ceramic-containing devices, the amount of ceramic (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device has two hydrogel compartments, in which the inner, cell-containing compartment is completely surrounded by the second, outer (e.g., barrier) compartment. In an embodiment, the inner boundary of the second compartment forms an interface with the outer boundary of the first compartment. In such embodiments, the thickness of the second (outer) compartment means the average distance between the outer boundary of the second compartment and the interface between the two compartments, e.g., the average of the distances measured at each of the thinnest and thickest points visually observed in the outer compartment. In some embodiments (e.g., the device is about 1.5 mm in diameter), the thinnest and thickest distances for the outer compartment are between 25 and 110 micrometers (μm) and between 270 and 480 μm, respectively. In some embodiments, the thickness of the outer compartment is greater than about 10 nanometers (nm), preferably 100 nm or greater and can be as large as 1 millimeter (mm). For example, the thickness (e.g., average distance) of the outer compartment in a hydrogel capsule device described herein may be 10 nm to 1 mm, 100 nm to 1 mm, 500 nm to 1 millimeter, 1 micrometer (μm) to 1 mm, 1 μm to 1 mm, 1 μm to 500 μm, 1 μm to 250 μm, 1 μm to 1 mm, 5 μm to 500 μm, 5 μm to 250 μm, 10 μm to 1 mm, 10 μm to 500 μm, or 10 μm to 250 μm. In some embodiments, the thickness (e.g., average distance) of the outer compartment is 100 nm to 1 mm, between 1 μm and 1 mm, between 1 μm and 500 μm or between 5 μm and 1 mm. In some embodiments, the thickness (e.g., average distance) of the outer compartment is between about 50 μm and about 100 μm. In some embodiments (e.g., the device is about 1.5 mm in diameter), the thickness of the outer compartment (e.g., average distance) is between about 180 μm and 260 μm or between about 310 μm and 440 μm.

In some embodiments of a two-compartment hydrogel capsule device, the mean pore size of the cell-containing inner compartment and the outer compartment is substantially the same. In some embodiments, the mean pore size of the inner compartment and the second compartment differ by about 1.5%, 2%, 5%, 7.5%, 10%, 15%, 20%, 25%, 30%, 35%, 40%, 45%, 50%, 55%, 60%, 65%, 70%, 75%, or more. In some embodiments, the mean pore size of the device (e.g., mean pore size of the first compartment and/or mean pore size of the second compartment) is dependent on a number of factors, such as the material(s) within each compartment and the presence and density of a compound of Formula (III).

In some embodiments, the polymer composition in the cell-containing compartment(s) comprises a polysaccharide or other hydrogel-forming polymer (e.g., alginate, hyaluronate or chondroitin). In some embodiments, the polymer is an alginate, which is a polysaccharide made up of β-D-mannuronic acid (M) and α-L-guluronic acid (G). In some embodiments, the alginate has a low molecular weight (e.g., approximate molecular weight of <75 kD) and G:M ratio ≥1.5, (ii) a medium molecular weight alginate, e.g., has approximate molecular weight of 75-150 kDa and G:M ratio ≥1.5, (iii) a high molecular weight alginate, e.g., has an approximate MW of 150 kDa-250 kDa and G:M ratio ≥1.5, (iv) or a blend of two or more of these alginates. In some embodiments, the cell-containing compartment(s) further comprises at least one cell-binding substance (CBS), e.g., a cell-binding peptide (CBP) or cell-binding polypeptide (CBPP) described in WO2020069429.

In some embodiments, the cell-containing compartment(s) comprises an alginate covalently modified with a linker-cell-binding peptide moiety, e.g., GRGD or GRGDSP. In an embodiment, the cell-binding peptide density in the cell-containing compartment(s) (% nitrogen as determined by combustion analysis, e.g., as described in WO2020198695) to be at least 0.05%, 0.1%, 0.2% or 0.3% but less than 4%, 3%, 2% or 1%. In an embodiment, the total density of the linker-CBP in a cell containing compartment is about 0.1 to about 1.0 micromoles of the CBP per g of CBP-polymer (e.g., a MMW-alginate covalently modified with GRGD (SEQ ID NO:88) or GRGDSP (SEQ ID NO:89) in solution as determined by a quantitative peptide conjugation assay, e.g., an assay described in WO2020198695. In an embodiment, the linker-CBP is GRGDSP and the alginate has a molecular weight of 75 kDa to 150 kDa and a G:M ratio of greater than or equal to 1.5. In an embodiment, the cell-containing compartment also comprises an unmodified alginate with a molecular weight of 75 kDa to 150 kDa and a G:M ratio of greater than or equal to 1.5.

The device may form part of a plurality of substantially the same devices in a preparation (e.g., composition). In some embodiments, the devices (e.g., particles, hydrogel capsules) in the preparation have a mean diameter or size between about 0.5 mm to about 8 mm. In some embodiments, the mean diameter or size of devices in the preparation is between about 0.5 mm to about 4 mm or between about 0.5 mm to about 2 mm. In some embodiments, the devices in the preparation are two-compartment hydrogel capsules and have a mean diameter or size of about 0.7 mm to about 1.3 mm or about 1.2 mm to about 1.8 mm.

In some embodiments, the surface of the device comprises a compound capable of mitigating the FBR upon implant into a subject, an afibrotic compound as described herein below. For devices comprising a barrier compartment surrounding the cell-containing compartment, the afibrotic compound may covalently modify a polymer disposed throughout the barrier compartment and optionally throughout the cell-containing compartment.

In some embodiments, one or more compartments in a device comprises an afibrotic polymer, e.g., an afibrotic compound of Formula (III) covalently attached to a polymer. In an embodiment, some or all the monomers in the afibrotic polymer are modified with the same compound of Formula (III). In some embodiments, some or all the monomers in the afibrotic polymer are modified with different compounds of Formula (III). In some embodiments in which the device is a 2-compartment hydrogel capsule, the afibrotic polymer is present only in the outer, barrier compartment.

One or more compartments in a device may comprise an unmodified polymer that is the same or different than the polymer in any afibrotic polymer that is present in the device. In an embodiment, the first compartment, second compartment or all compartments in the device comprise the unmodified polymer.

Each of the modified and unmodified polymers in the device may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, ladders, and dendrimers. A polymer may be a thermoresponsive polymer, e.g., gel (e.g., becomes a solid or liquid upon exposure to heat or a certain temperature) or a photocrosslinkable polymer. Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, PEEK®, Teflon® (polytetrafluoroethylene, PTFE), PEEK, silicones, epoxy resins, Kevlar®, Dacron® (a condensation polymer obtained from ethylene glycol and terephthalic acid), polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), fluoroplastics, carbon fiber, agarose, alginate, chitosan, and blends or copolymers thereof. In polymer-containing devices, the amount of a polymer (e.g., by % weight of the device, actual weight of the polymer) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, one or more of the modified and unmodified polymers in the device comprises a polyethylene. Exemplary polyethylenes include ultra-low-density polyethylene (ULDPE) (e.g., with polymers with densities ranging from 0.890 to 0.905 g/cm³, containing comonomer); very-low-density polyethylene (VLDPE) (e.g., with polymers with densities ranging from 0.905 to 0.915 g/cm³, containing comonomer); linear low-density polyethylene (LLDPE) (e.g., with polymers with densities ranging from 0.915 to 0.935 g/cm³, contains comonomer); low-density polyethylene (LDPE)(e.g., with polymers with densities ranging from about 0.915 to 0.935 g/m³); medium density polyethylene (MDPE) (e.g., with polymers with densities ranging from 0.926 to 0.940 g/cm³, may or may not contain comonomer); high-density polyethylene (HDPE) (e.g., with polymers with densities ranging from 0.940 to 0.970 g/cm³, may or may not contain comonomer) and polyethylene glycol.

In some embodiments, one or more of the modified and unmodified polymers in the device comprises a polypropylene. Exemplary polypropylenes include homopolymers, random copolymers (homophasic copolymers), and impact copolymers (heterophasic copolymers), e.g., as described in McKeen, Handbook of Polymer Applications in Medicine and Medical Devices, 3-Plastics Used in Medical Devices, (2014):21-53.

In some embodiments, one or more of the modified and unmodified polymers in the device comprises a polypropylene. Exemplary polystyrenes include general purpose or crystal (PS or GPPS), high impact (HIPS), and syndiotactic (SPS) polystyrene.

In some embodiments, one or more of the modified and unmodified polymers comprises a comprises a thermoplastic elastomer (TPE). Exemplary TPEs include (i) TPA-polyamide TPE, comprising a block copolymer of alternating hard and soft segments with amide chemical linkages in the hard blocks and ether and/or ester linkages in the soft blocks; (ii) TPC-co-polyester TPE, consisting of a block copolymer of alternating hard segments and soft segments, the chemical linkages in the main chain being ester and/or ether; (iii) TPO-olefinic TPE, consisting of a blend of a polyolefin and a conventional rubber, the rubber phase in the blend having little or no cross-linking; (iv) TPS-styrenic TPE, consisting of at least a triblock copolymer of styrene and a specific diene, where the two end blocks (hard blocks) are polystyrene and the internal block (soft block or blocks) is a polydiene or hydrogenated polydiene; (v) TPU-urethane TPE, consisting of a block copolymer of alternating hard and soft segments with urethane chemical linkages in the hard blocks and ether, ester or carbonate linkages or mixtures of them in the soft blocks; (vi) TPV-thermoplastic rubber vulcanizate consisting of a blend of a thermoplastic material and a conventional rubber in which the rubber has been cross-linked by the process of dynamic vulcanization during the blending and mixing step; and (vii) TPZ-unclassified TPE comprising any composition or structure other than those grouped in TPA, TPC, TPO, TPS, TPU, and TPV.

In some embodiments, the unmodified polymer is an unmodified alginate. In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In an embodiment, the unmodified alginate has a molecular weight of 150 kDa-250 kDa and a G:M ratio of ≥1.5.

In some embodiments, the afibrotic polymer comprises an alginate chemically modified with a Compound of Formula (III). The alginate in the afibrotic polymer may be the same or different than any unmodified alginate that is present in the device. In an embodiment, the density of the Compound of Formula (III) in the afibrotic alginate (e.g., amount of conjugation) is between about 4.0% and about 8.0%, between about 5.0% and about 7.0%, or between about 6.0% and about 7.0% nitrogen (e.g., as determined by combustion analysis for percent nitrogen). In an embodiment, the amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis and corresponds to the amount of Compound 101 in the modified alginate.

In other embodiments, the density (e.g., concentration) of the Compound of Formula (III) (e.g., Compound 101) in the afibrotic alginate is defined as the % w/w, e.g., % of weight of amine/weight of afibrotic alginate in solution (e.g., saline) as determined by a suitable quantitative amine conjugation assay (e.g. by an assay described in WO2020069429), and in certain embodiments, the density of a Compound of Formula (III) (e.g., Compound 101) is between about 1.0% w/w and about 3.0% w/w, between about 1.3% w/w and about 2.5% w/w or between about 1.5% w/w and 2.2% w/w.

In alginate-containing devices, the amount of modified and unmodified alginates (e.g., by % weight of the device, actual weight of the alginate) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

The alginate in an afibrotic polymer can be chemically modified with a compound of Formula (III) using any suitable method known in the art. For example, the alginate carboxylic acid moiety can be activated for coupling to one or more amine-functionalized compounds to achieve an alginate modified with a compound of Formula (III). The alginate polymer may be dissolved in water (30 mL/gram polymer) and treated with an activating agent (e.g., 2-chloro-4,6-dimethoxy-1,3,5-triazine (0.5 eq)) and a base (e.g., N-methylmorpholine (1 eq)). To this mixture may be added a solution of the compound of Formula (III) in acetonitrile (0.3M). The reaction may be warmed to 55° C. for 16 h, then cooled to room temperature and gently concentrated via rotary evaporation, then the residue may be dissolved, e.g., in water. The mixture may then be filtered, e.g., through a bed of cyano-modified silica gel (Silicycle) and the filter cake washed with water. The resulting solution may then be dialyzed (10,000 MWCO membrane) against water for 24 hours, e.g., replacing the water twice. The resulting solution can be concentrated, e.g., via lyophilization, to afford the desired chemically modified alginate. The alginate in an afibrotic polymer can be chemically modified with a compound of Formula (III) using any suitable method known in the art, e.g., as described in any of WO 2021/119522, WO 2019/195055, WO 2018/067615, WO 2017/075631, WO 2016/019391 and WO 2012/167223.

In an embodiment, the device comprises at least one cell-containing compartment, and in some embodiments contains two, three, four or more cell-containing compartments. In an embodiment, each cell-containing compartment comprises a plurality of cells (e.g., live cells) and the cells in at least one of the compartments are capable of expressing and secreting a BBB-transporting fusion protein when the device is implanted into a subject.

In an embodiment, all the cells in a cell-containing compartment are derived from a single parental cell-type or a mixture of at least two different parental cell types. In an embodiment, all of the cells in a cell-containing compartment are derived from the same parental cell type, but a first plurality of the derived cells are engineered to express the BBB-transporting fusion protein, and a second plurality of the derived cells are engineered to express a different therapeutic protein. In devices with two or more cell-containing compartments, the cells and the protein(s) produced thereby may be the same or different in each cell-containing compartment. In some embodiments, all of the cell-containing compartments are surrounded by a single barrier compartment. In some embodiments, the barrier compartment is substantially cell-free.

In an embodiment, cells to be incorporated into a device described herein, e.g., a hydrogel capsule, are prepared in the form of a cell suspension prior to being encapsulated within the device. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In addition to the fusion protein secreted by the encapsulated cells, a device (e.g., capsule, particle) may comprise one or more exogenous agents that are not expressed by the cells, and may include, e.g., a nucleic acid (e.g., an RNA or DNA molecule), a protein (e.g., a hormone, an enzyme (e.g., glucose oxidase, kinase, phosphatase, oxygenase, hydrogenase, reductase) antibody, antibody fragment, antigen, or epitope)), an active or inactive fragment of a protein or polypeptide, a small molecule, or drug. In an embodiment, the device is configured to release such an exogenous agent.

### Afibrotic (e.g., FBR-Mitigating) Compounds

In some embodiments, the devices described herein comprise at least one compound of Formula (III):

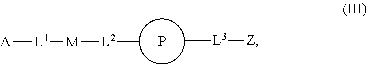

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), N(R^{C})C(O)R^{A}, —N(R^{C})(R^{D}), cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more RV;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, OS(O)ₓR^{E1}, N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, the compound of Formula (III) is a compound of Formula (III-a):

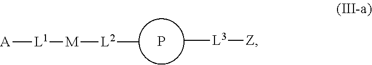

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, N(R^{C})C(O)(C₁-C₆-alkylene)-, —N(R^{C})C(O)(C₁-C₆-alkenylene)-, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, OS(O)ₓR^{E1}, N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, for Formulas (III) and (III-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{C})—, and R^{C} an R^{D} is independently hydrogen or alkyl. In some embodiments, A is —NH—. In some embodiments, A is —N(R^{C})C(O)(C₁-C₆-alkylene), wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (III) and (III-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (III) and (III-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (III) and (III-a), M is absent, alkyl, heteroalkyl, aryl, or heteroaryl. In some embodiments, for Formulas (III) and (III-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂—)_{z}, wherein z is an integer selected from 1 to 10. In some embodiments, z is an integer selected from 1 to 5. In some embodiments, M is —(OCH₂)₂—, (—OCH₂CH₂—)₂, (—OCH₂CH₂—)₃, (—OCH₂CH₂—)₄, or (—OCH₂CH₂—)₅. In some embodiments, M is —OCH₂CH₂—, (—OCH₂CH₂—)₂, (—OCH₂CH₂—)₃, or (—OCH₂CH₂—)₄. In some embodiments, M is (—OCH₂—)₃. In some embodiments, M is aryl. In some embodiments, M is phenyl. In some embodiments, M is unsubstituted phenyl. In some embodiments, M is

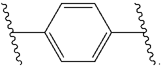 [SMILES: *c1ccc(*)cc1]

In some embodiments, M is

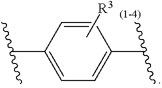

In some embodiments, M is phenyl substituted with 1-4 R³ (e.g., 1 R³). In some embodiments, R³ is CF₃.

In some embodiments, for Formulas (III) and (III-a), P is absent, heterocyclyl, or heteroaryl. In some embodiments, for Formulas (III) and (III-a), P is absent, heterocyclyl, or heteroaryl. In some embodiments, P is absent. In some embodiments, for Formulas (III) and (III-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, or pyrrolyl. In some embodiments, P is imidazolyl. In some embodiments, P is 1,2,3-triazolyl. In some embodiments, P is

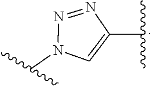 [SMILES: *c1cn(*)nn1]

In some embodiments, P is

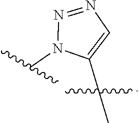 [SMILES: *c1cnnn1*]

In some embodiments, P is

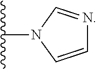 [SMILES: *n1ccnc1]

In some embodiments, P is heterocyclyl. In some embodiments, P is heterocyclyl. In some embodiments, P is a 5-membered heterocyclyl. In some embodiments, P is imidazolidinonyl. In some embodiments, P is

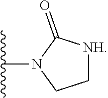 [SMILES: *N1CCNC1=O]

In some embodiments, P is thiomorpholinyl-1,1-dioxidyl. In some embodiments, P is

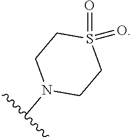

In some embodiments, for Formulas (III) and (III-a), Z is alkyl, heteroalkyl, cycloalkyl, heterocyclyl, aryl, or heteroaryl. In some embodiments, for Formulas (III) and (III-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl, 5-membered heterocyclyl, or 6-membered heterocyclyl. In some embodiments, Z is a 6-membered oxygen-containing heterocyclyl. In some embodiments, Z is tetrahydropyranyl. In some embodiments, Z is

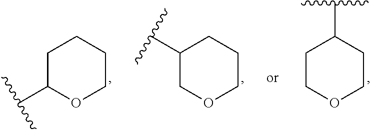 [SMILES: *C1CCCCO1 ; *C1CCCOC1 ; *C1CCOCC1]

In some embodiments, Z is a 4-membered oxygen-containing heterocyclyl. In some embodiments, Z is

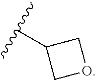 [SMILES: *C1COC1]

In some embodiments, Z is a bicyclic oxygen-containing heterocyclyl. In some embodiments, Z is a bicyclic oxygen-containing heterocyclyl. In some embodiments, Z is phthalic anhydridyl. In some embodiments, Z is a sulfur-containing heterocyclyl

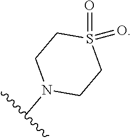

In some embodiments, Z is a 6-membered sulfur-containing heterocyclyl

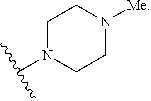 [SMILES: *N1CCN(C)CC1]

In some embodiments, Z is a 6-membered heterocyclyl containing a nitrogen atom and a sulfur atom. In some embodiments, Z is thiomorpholinyl-1,1-dioxidyl. In some embodiments, Z is

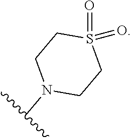

In some embodiments, Z is a nitrogen-containing heterocyclyl. In some embodiments, Z is a 6-membered nitrogen-containing heterocyclyl. In some embodiments, Z is

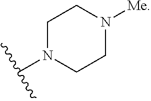 [SMILES: *N1CCN(C)CC1]

In some embodiments, Z is a bicyclic heterocyclyl. In some embodiments, Z is a bicyclic heterocyclyl In some embodiments, Z is a bicyclic nitrogen-containing heterocyclyl, optionally substituted with one or more R⁵. In some embodiments, Z is 2-oxa-7-azaspiro[3.5]nonanyl In some embodiments, Z is

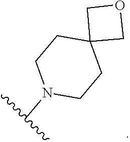

In some embodiments, Z is 1-oxa-3,8-diazaspiro[4.5]decan-2-one. In some embodiments, Z is

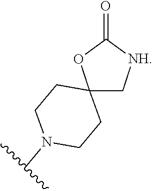

In some embodiments, for Formulas (III) and (III-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (III) and (III-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 RV, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 RV, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (III) and (III-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (III) is a compound of Formula (III-b):

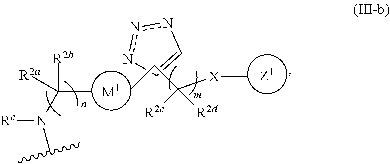

or a pharmaceutically acceptable salt thereof, wherein Ring M¹ is cycloalkyl, heterocyclyl, aryl, or heteroaryl, each of which is optionally substituted with 1-5 R³; Ring Z¹ is cycloalkyl, heterocyclyl aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (III-b) is a compound of Formula (III-b-i):

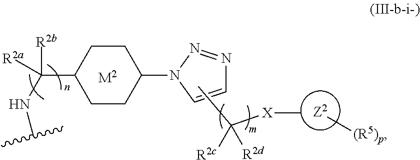

or a pharmaceutically acceptable salt thereof, wherein Ring M² is aryl or heteroaryl optionally substituted with one or more R³; Ring Z² is cycloalkyl, heterocyclyl, aryl- or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (III-b-i) is a compound of Formula (III-b-ii):

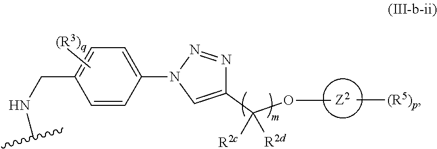

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (III) is a compound of Formula (III-c):

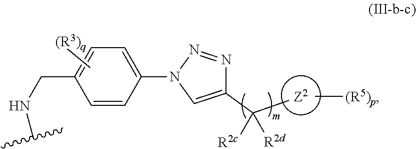

or a pharmaceutically acceptable salt thereof, wherein Ring Z² is cycloalkyl, heterocyclyl aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR, —C(O)OR, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (III) is a compound of Formula (III-d):

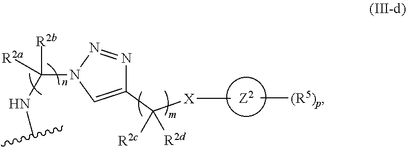

or a pharmaceutically acceptable salt thereof, wherein Ring Z² is cycloalkyl, heterocyclyl aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (III) is a compound of Formula (III-e):

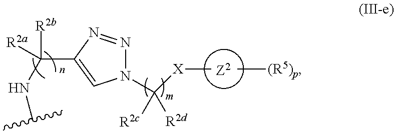

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (III) is a compound of Formula (III-f):

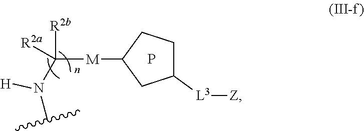

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (III) is a compound of Formula (IV):

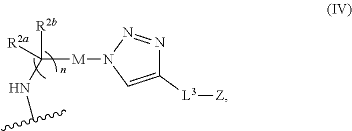

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR, wherein alkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (IV) is a compound of Formula (IV-a):

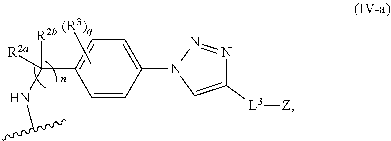

or a pharmaceutically acceptable salt thereof, wherein L³ is alkyl or heteroalkyl, each of which is optionally substituted with one or more R²; Z is hydrogen, alkyl, heteroalkyl- or —OR^{A}, heteroalkyl are optionally substituted with one or more R⁵; each of R^{2a} and R^{2b} is independently hydrogen, alkyl, or heteroalkyl, or each of R^{2a} and R^{2b} is taken together to form an oxo group each R², R³, and R⁵ is independently heteroalkyl, halogen, oxo, —OR^{A1}, —C(O)OR^{A1}; R^{A} is hydrogen; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (III) is a compound of Formula (V):

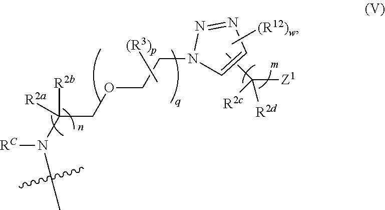

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or each of R^{2a} and R^{2b} or R^{2c} and R^{2d} is taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; w is 0 or 1; and “ [symbol] ” refers to a connection to an attachment group or a polymer described herein.

In some embodiments, the compound of Formula (V) is a compound of Formula (V-a):

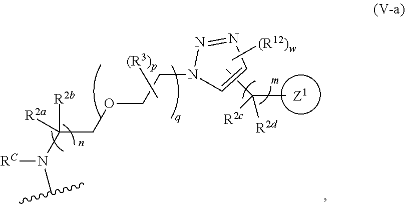

or a pharmaceutically acceptable salt thereof, wherein Ring Z¹ is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; w is 0 or 1; and “ [symbol] ” refers to a connection to an attachment group or a polymer described herein.

In some embodiments, the compound of Formula (V) is a compound of Formula (V-b):

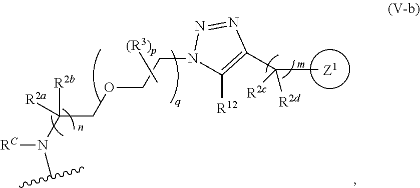

or a pharmaceutically acceptable salt thereof, wherein Ring Z¹ is cycloalkyl, heterocyclyl, aryl, or heteroaryl, each of which is optionally substituted with 1-5 R⁵; R^{C} is hydrogen, alkyl, —N(R^{C})C(O)R^{B}, N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; and “ [symbol] ” refers to a connection to an attachment group or a polymer described herein.

In some embodiments, the compound of Formula (V) is a compound of Formula (V-c):

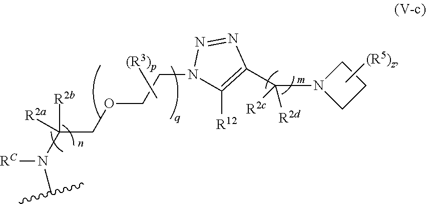

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, the compound of Formula (V) is a compound of Formula (V-d):

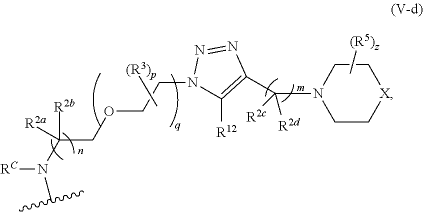

or a pharmaceutically acceptable salt thereof, wherein X is C(R′)(R″), N(R′), or S(O)ₓ; each of R′ and R″ is independently hydrogen, alkyl, or halogen; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a [symbol] connection to an attachment group or a polymer described herein.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹² is halo (e.g., Cl).

In some embodiments, the compound is a compound of Formula (III). In some embodiments, L² is a bond and P and L³ are independently absent.

In some embodiments, the compound is a compound of Formula (III-a). In some embodiments of Formula (IV-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (III-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (III-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (III-b) is Compound 105.

In some embodiments, the compound is a compound of Formula (III-b-i). In some embodiments of Formula (III-b-i), each of R^{2a} and R^{2b} is independently hydrogen or CH₃, each of R^{2c} and R^{2d} is independently hydrogen, m is 1 or 2, n is 1, X is O, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (III-b-i) is Compound 100, Compound 106, Compound 107, Compound 108, Compound 109, or Compound 111.

In some embodiments, the compound is a compound of Formula (III-b-ii). In some embodiments of Formula (III-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (III-b-ii) is Compound 100.

In some embodiments, the compound is a compound of Formula (III-c). In some embodiments of Formula (III-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (III-d). In some embodiments of Formula (III-d), each of R^{2a}, R^{2b}, R^{2c}, and R^{2d} is independently hydrogen, m is 1, n is 3, X is O, p is 0, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (III-d) is Compound 110 or Compound 114.

In some embodiments, the compound is a compound of Formula (III-f). In some embodiments of Formula (III—), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (III-f) is Compound 115.

In some embodiments, the compound is a compound of Formula (IV-a). In some embodiments of Formula (IV-a), each of R^{2a} and R^{2b} is independently hydrogen, n is 1, q is 0, L³ is —CH₂(OCH₂CH₂)₂, and Z is —OCH₃. In some embodiments, the compound of Formula (IV-a) is Compound 112.

In some embodiments of Formula (IV-a), each of R^{2a} and R^{2b} is independently hydrogen, n is 1, L³ is a bond or —CH₂, and Z is hydrogen or —OH. In some embodiments, the compound of Formula (IV-a) is Compound 103 or Compound 104.

In some embodiments, the compound is a compound of Formula (V). In some embodiments of Formula (V), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (V) is Compound 120.

In some embodiments, the compound is a compound of Formula (V-b). In some embodiments of Formula (V-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (V-b). In some embodiments of Formula (V-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., a nitrogen-containing heterocyclyl, e.g., a nitrogen-containing spiro heterocyclyl, e.g., 2-oxa-7-azaspiro[3.5]nonanyl). In some embodiments, the compound of Formula (V-b) is Compound 121.

In some embodiments, the compound is a compound of Formula (V-d). In some embodiments of Formula (V-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (V-d), each of R^{2a} and R^{2b} is independently hydrogen, m is 1, n is 2, q is 1, 2, 3, or 4, p is 0, and X is S(O)₂. In some embodiments, the compound of Formula (V-d) is Compound 101, Compound 117, Compound 118, or Compound 119.

In some embodiments, the compound is a compound of Formula (III-b), (III-d), or (III-e). In some embodiments, the compound is a compound of Formula (III-b), (III-d), or (IV). In some embodiments, the compound is a compound of Formula (III-b), (III-d), or (III-f). In some embodiments, the compound is a compound of Formula (III-b), (III-d), or (V).

In some embodiments, the compound of Formula (I) is not a compound disclosed in WO2012/112982, WO2012/167223, WO2014/153126, WO2016/019391, WO 2017/075630, US2012-0213708, US 2016-0030359 or US 2016-0030360.

In some embodiments, the compound of Formula (III) comprises a compound shown in Table 6 herein below, or a pharmaceutically acceptable salt thereof. In some embodiments, the exterior surface and/or one or more compartments within a device described herein comprises a small molecule compound shown in Table 6, or a pharmaceutically acceptable salt thereof.

TABLE 6
Exemplary afibrotic (FBR-mitigating) compounds

| Compound No. | Structure |
|---|---|
| 100 | 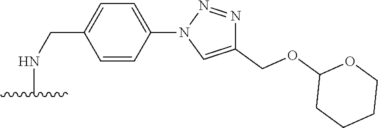 [SMILES: *NCc1ccc(-n2cc(COC3CCCCO3)nn2)cc1] |
| 101 | 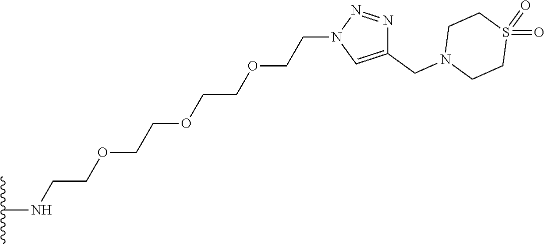 [SMILES: *NCCOCCOCCOCCn1cc(CN2CCS(=O)(=O)CC2)nn1] |
| 102 | 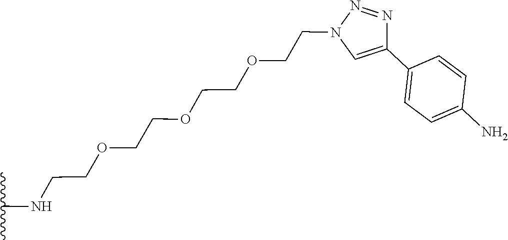 [SMILES: *NCCOCCOCCOCCn1cc(-c2ccc(N)cc2)nn1] |
| 103 | 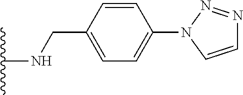 [SMILES: *NCc1ccc(-n2ccnn2)cc1] |
| 104 | 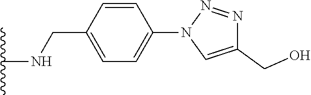 [SMILES: *NCc1ccc(-n2cc(CO)nn2)cc1] |
| 105 | 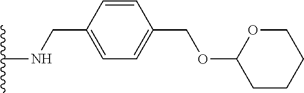 [SMILES: *NCc1ccc(COC2CCCCO2)cc1] |
| 106 | 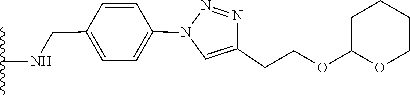 [SMILES: *NCc1ccc(-n2cc(CCOC3CCCCO3)nn2)cc1] |
| 107 | 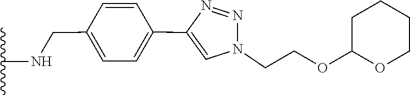 [SMILES: *NCc1ccc(-c2cn(CCOC3CCCCO3)nn2)cc1] |
| 108 | 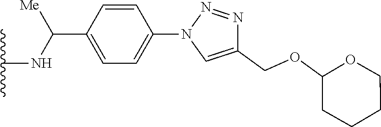 [SMILES: *NC(C)c1ccc(-n2cc(COC3CCCCO3)nn2)cc1] |
| 109 | 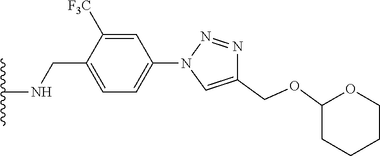 [SMILES: *NCc1ccc(-n2cc(COC3CCCCO3)nn2)cc1C(F)(F)F] |
| 110 | 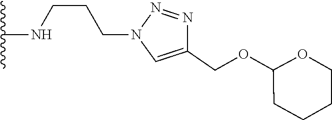 [SMILES: *NCCCn1cc(COC2CCCCO2)nn1] |
| 111 | 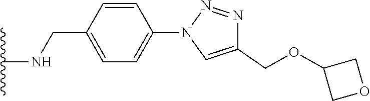 [SMILES: *NCc1ccc(-n2cc(COC3COC3)nn2)cc1] |
| 112 | 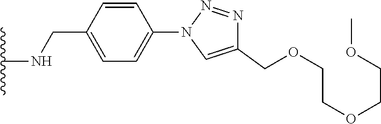 [SMILES: *NCc1ccc(-n2cc(COCCOCCOC)nn2)cc1] |
| 113 | 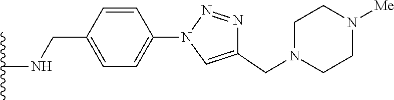 [SMILES: *NCc1ccc(-n2cc(CN3CCN(C)CC3)nn2)cc1] |
| 114 | 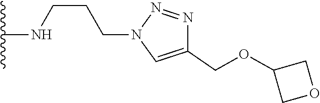 [SMILES: *NCCCn1cc(COC2COC2)nn1] |
| 115 | 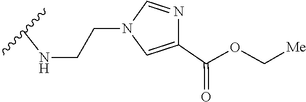 [SMILES: *NCCn1cnc(C(=O)OCC)c1] |
| 116 | 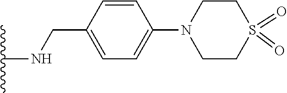 [SMILES: *NCc1ccc(N2CCS(=O)(=O)CC2)cc1] |
| 117 | 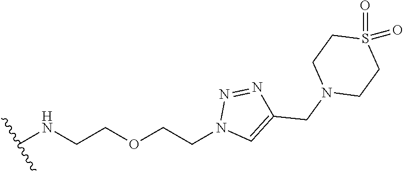 [SMILES: *NCCOCCn1cc(CN2CCS(=O)(=O)CC2)nn1] |
| 118 | 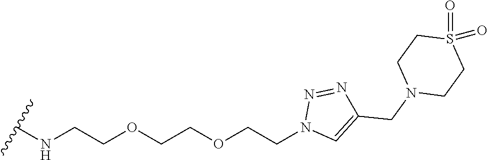 [SMILES: *NCCOCCOCCn1cc(CN2CCS(=O)(=O)CC2)nn1] |
| 119 | 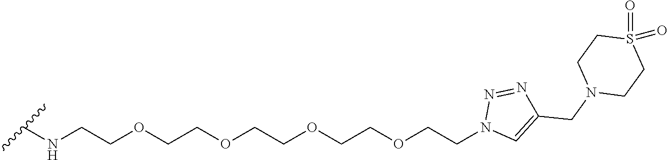 [SMILES: *NCCOCCOCCOCCOCCn1cc(CN2CCS(=O)(=O)CC2)nn1] |
| 120 | 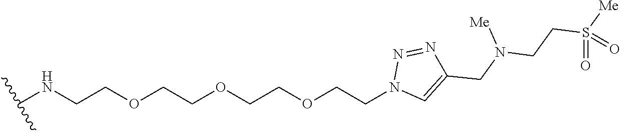 [SMILES: *NCCOCCOCCOCCn1cc(CN(C)CCS(C)(=O)=O)nn1] |
| 121 | 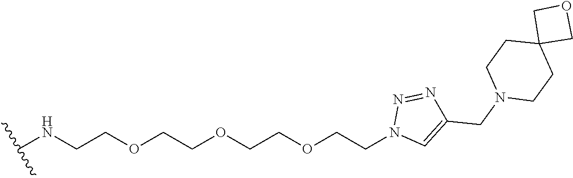 [SMILES: *NCCOCCOCCOCCn1cc(CN2CCC3(CC2)COC3)nn1] |
| 122 | 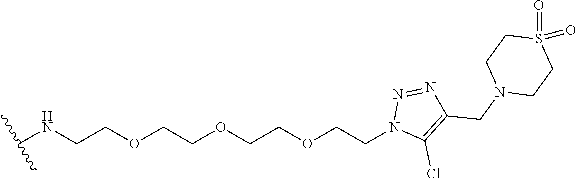 [SMILES: *NCCOCCOCCOCCn1nnc(CN2CCS(=O)(=O)CC2)c1Cl] |
| 123 | 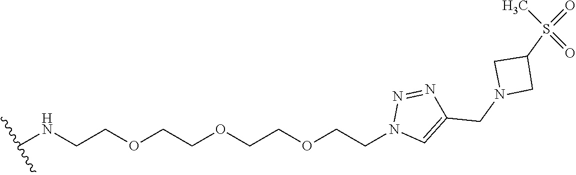 [SMILES: *NCCOCCOCCOCCn1cc(CN2CC(S(C)(=O)=O)C2)nn1] |
| 124 | 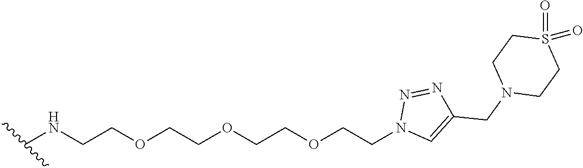 [SMILES: *NCCOCCOCCOCCn1cc(CN2CCS(=O)(=O)CC2)nn1] |
| 125 | 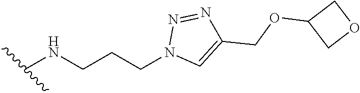 [SMILES: *NCCCn1cc(COC2COC2)nn1] |
| 126 | 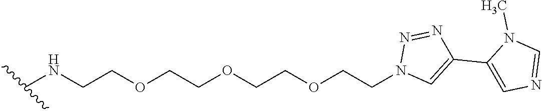 [SMILES: *NCCOCCOCCOCCn1cc(-c2cncn2C)nn1] |
| 127 | 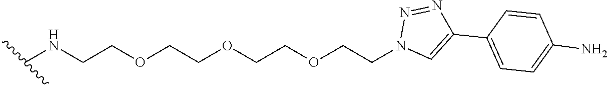 [SMILES: *NCCOCCOCCOCCn1cc(-c2ccc(N)cc2)nn1] |
| 128 | 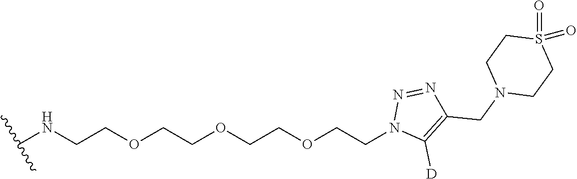 [SMILES: *NCCOCCOCCOCCn1nnc(CN2CCS(=O)(=O)CC2)c1[2H]] |
| 129 | 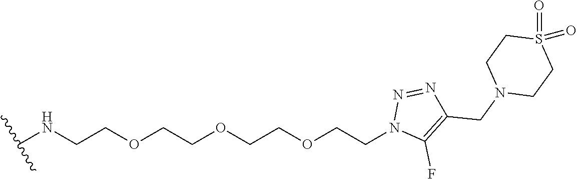 [SMILES: *NCCOCCOCCOCCn1nnc(CN2CCS(=O)(=O)CC2)c1F] |
| 130 | 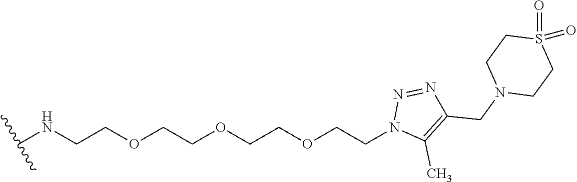 [SMILES: *NCCOCCOCCOCCn1nnc(CN2CCS(=O)(=O)CC2)c1C] |
| 131 | 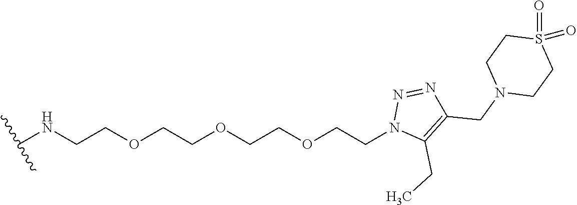 [SMILES: *NCCOCCOCCOCCn1nnc(CN2CCS(=O)(=O)CC2)c1CC] |
| 132 | 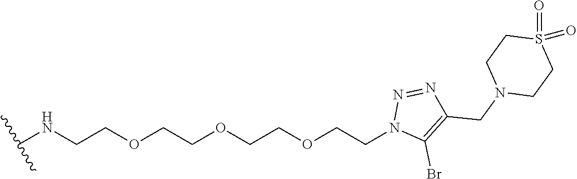 [SMILES: *NCCOCCOCCOCCn1nnc(CN2CCS(=O)(=O)CC2)c1Br] |
| 133 | 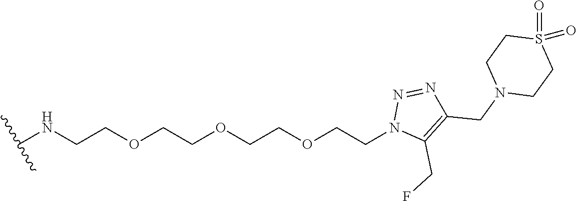 [SMILES: *NCCOCCOCCOCCn1nnc(CN2CCS(=O)(=O)CC2)c1CF] |
| 134 | 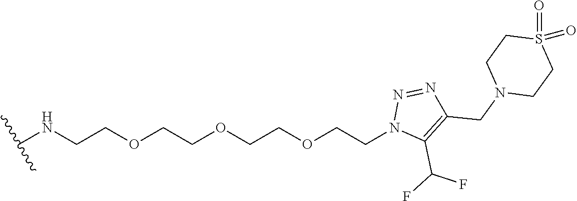 [SMILES: *NCCOCCOCCOCCn1nnc(CN2CCS(=O)(=O)CC2)c1C(F)F] |
| 135 | 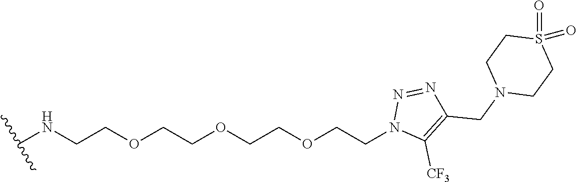 [SMILES: *NCCOCCOCCOCCn1nnc(CN2CCS(=O)(=O)CC2)c1C(F)(F)F] |
| 136 | 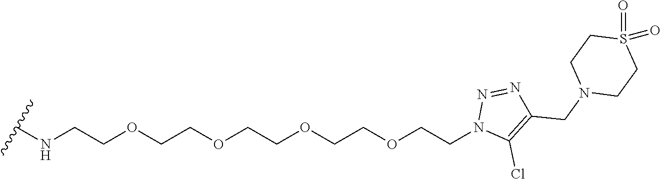 [SMILES: *NCCOCCOCCOCCOCCn1nnc(CN2CCS(=O)(=O)CC2)c1Cl] |
| 137 | 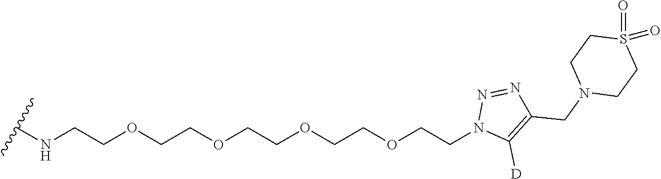 [SMILES: *NCCOCCOCCOCCOCCn1nnc(CN2CCS(=O)(=O)CC2)c1[2H]] |
| 138 | 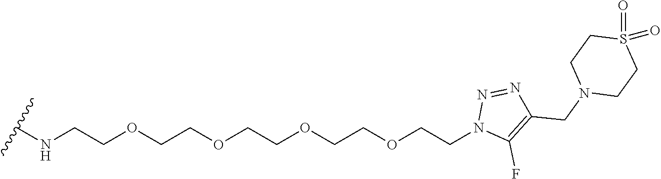 [SMILES: *NCCOCCOCCOCCOCCn1nnc(CN2CCS(=O)(=O)CC2)c1F] |
| 139 | 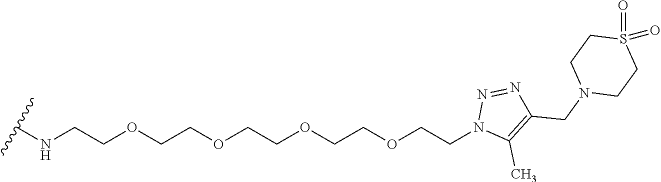 [SMILES: *NCCOCCOCCOCCOCCn1nnc(CN2CCS(=O)(=O)CC2)c1C] |
| 140 | 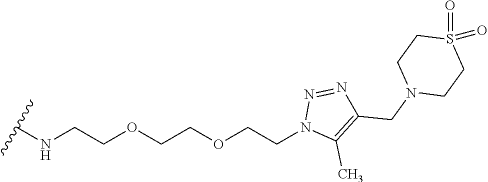 [SMILES: *NCCOCCOCCn1nnc(CN2CCS(=O)(=O)CC2)c1C] |
| 141 | 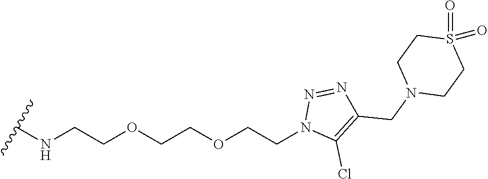 [SMILES: *NCCOCCOCCn1nnc(CN2CCS(=O)(=O)CC2)c1Cl] |
| 142 | 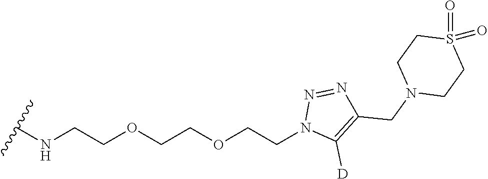 [SMILES: *NCCOCCOCCn1nnc(CN2CCS(=O)(=O)CC2)c1[2H]] |
| 143 | 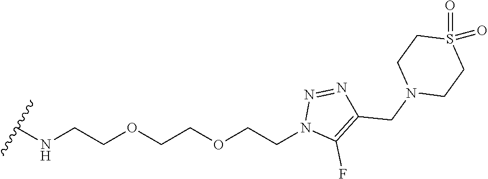 [SMILES: *NCCOCCOCCn1nnc(CN2CCS(=O)(=O)CC2)c1F] |
| 144 | 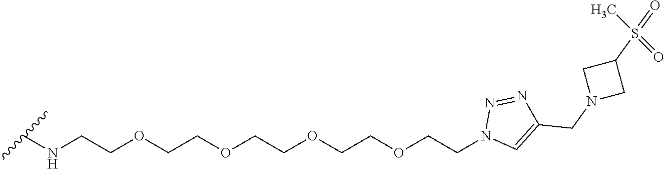 [SMILES: *NCCOCCOCCOCCOCCn1cc(CN2CC(S(C)(=O)=O)C2)nn1] |
| 145 | 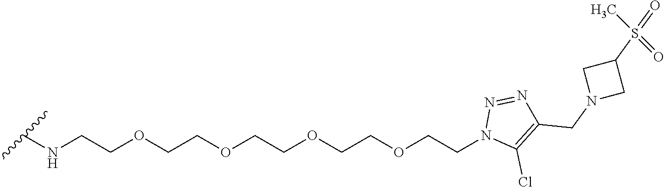 [SMILES: *NCCOCCOCCOCCOCCn1nnc(CN2CC(S(C)(=O)=O)C2)c1Cl] |
| 146 | 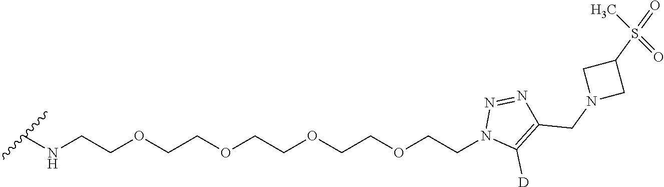 [SMILES: *NCCOCCOCCOCCOCCn1nnc(CN2CC(S(C)(=O)=O)C2)c1[2H]] |
| 147 | 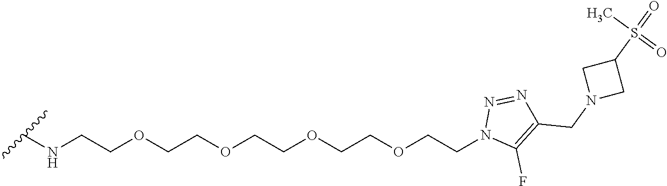 [SMILES: *NCCOCCOCCOCCOCCn1nnc(CN2CC(S(C)(=O)=O)C2)c1F] |
| 148 | 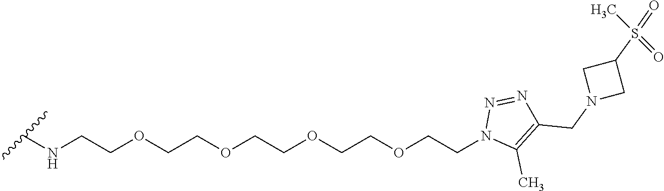 [SMILES: *NCCOCCOCCOCCOCCn1nnc(CN2CC(S(C)(=O)=O)C2)c1C] |
| 149 | 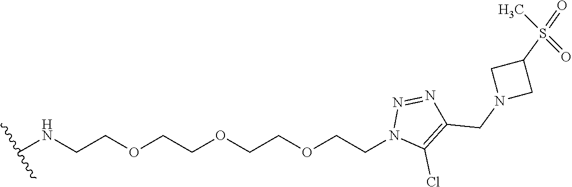 [SMILES: *NCCOCCOCCOCCn1nnc(CN2CC(S(C)(=O)=O)C2)c1Cl] |
| 150 | 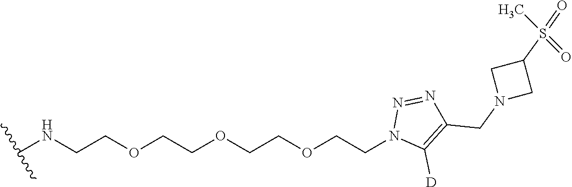 [SMILES: *NCCOCCOCCOCCn1nnc(CN2CC(S(C)(=O)=O)C2)c1[2H]] |
| 151 | 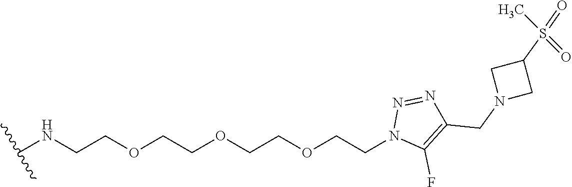 [SMILES: *NCCOCCOCCOCCn1nnc(CN2CC(S(C)(=O)=O)C2)c1F] |
| 152 | 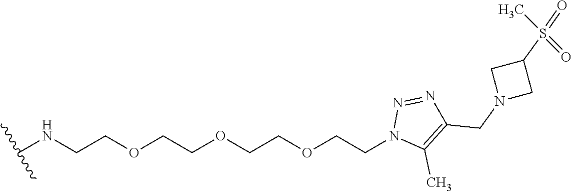 [SMILES: *NCCOCCOCCOCCn1nnc(CN2CC(S(C)(=O)=O)C2)c1C] |
| 153 | 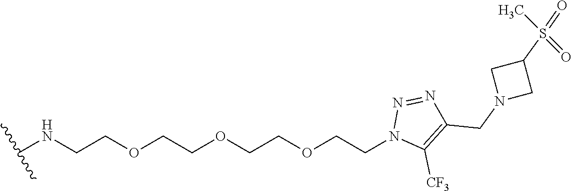 [SMILES: *NCCOCCOCCOCCn1nnc(CN2CC(S(C)(=O)=O)C2)c1C(F)(F)F] |
| 154 | 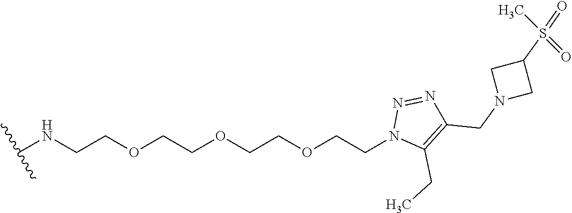 [SMILES: *NCCOCCOCCOCCn1nnc(CN2CC(S(C)(=O)=O)C2)c1CC] |

Conjugation of any of the compounds in Table 6 to a polymer (e.g., an alginate) may be performed as described in Example 2 of WO 2019/195055 or in or any other suitable chemical reaction.

In some embodiments, the compound is a compound of Formula (III or a pharmaceutically acceptable salt thereof and is selected from:

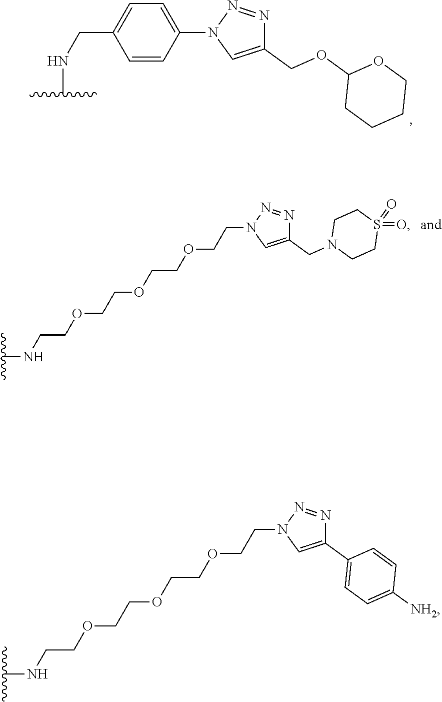 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt thereof.

In some embodiments, the device described herein comprises the compound of

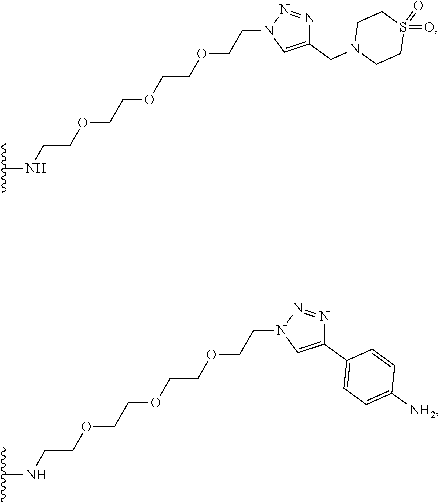 [SMILES: *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt of either compound.

In some embodiments, a compound of Formula (III) (e.g., Compound 101 in Table 6) is covalently attached to an alginate (e.g., an alginate with approximate MW <75 kDa, G:M ratio ≥1.5) at a conjugation density of at least 2.0% and less than 9.0%, or 3.0% to 8.0%, 4.0-7.0, 5.0 to 7.0, or 6.0 to 7.0 or about 6.8 as determined by combustion analysis for percent nitrogen as described in WO 2020/069429. In an embodiment, the conjugation density of Compound 101 in the modified alginate is determined by quantitative free amine analysis, e.g., as described in WO2020198695, wherein the determined conjugation density is 1.0% w/w to 3.0% w/w, 1.3% w/w to 2.8% w/w, 1.3% w/w to 2.6% w/w, 1.5% w/w to 2.4% w/w, 1.5% w/w to 2.2% w/w, or 1.7% w/w to 2.2% w/w.

A device, device preparation or device composition may be configured for implantation, or is implanted or disposed, into or onto any site or part of the body. In some embodiments, the implantable device or device preparation is configured for implantation into the peritoneal cavity (e.g., the lesser sac, also known as the omental bursa or bursalis omentum). A device, device preparation or device composition may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of a device, device preparation or device composition into the omentum (e.g., the lesser sac) are provided in M. Pellicciaro et al. (2017) Cell R4 5(3):e2410.

### Device Manufacture

Genetically modified ARPE-19 cells for use in manufacturing a device described herein may be generated and cultured using methods known in the art. For example, stably-transfected ARPE-19 cells may be cultured in vitro substantially as described in WO2020198695.

Compounds of Formula (III) and alginates modified with such compounds may be obtained using procedures known in the art, e.g., substantially as those described in WO2020198695.

Alginate solutions for making afibrotic, two-compartment hydrogel capsules may be obtained using procedures known in the art, e.g., substantially as described in WO2020198695.

Two-compartment hydrogel capsules encapsulating in the inner compartment genetically modified mammalian cells and an afibrotic alginate in the outer layer (“shielded capsules”) may be generated using procedures known in the art, e.g., substantially as described in WO2020198696.

### Methods of Treatment

Described herein are methods for preventing or treating a CNS disease or condition in a subject (e.g., a lysosomal storage disease, e.g., MPS-1) through administration or implantation of a pharmaceutical composition or genetically modified cells described herein. In an embodiment, the pharmaceutical composition comprises a BBB-transporting fusion protein and is formulated for intravenous or subcutaneous administration. In another embodiment, the pharmaceutical composition comprises a plurality of cells that are genetically modified to express the BBB-transporting fusion protein. In an embodiment, the cells are encapsulated in hydrogel capsules described herein. In another embodiment, the cells are encapsulated in a macrodevice described herein. In some embodiments, the methods described herein directly or indirectly reduce or alleviate at least one symptom of the CNS disease or condition, or prevent or slow the onset of the disease. In an embodiment, the method comprises administering (e.g., implanting) an effective amount of a composition of two-compartment alginate hydrogel capsules which comprise in the inner compartment genetically modified RPE cells and a cell-binding polymer described herein and comprise a Compound of Formula (III), e.g., Compound 101, on the outer capsule surface and optionally within the outer compartment.

### Treating MPS-1

The present disclosure provides a method of treating a human patient with MPS-1 by administering to the patient a BBB-transporting IDUA fusion protein described herein. In an embodiment, the administering step comprises implanting cells engineered to express and secrete the BBB-transporting IDUA fusion protein, which cells may be encapsulated in a macrodevice or in two-compartment alginate hydrogel capsules described herein. In an embodiment, encapsulated cells expressing the BBB-transporting IDUA fusion protein are implanted into the peritoneal cavity (e.g., the lesser sac, also known as the omental bursa or bursalis omentum). In an embodiment, the BBB-transporting IDUA fusion protein expressed and secreted by the implanted cells comprises SEQ ID NO:28 or SEQ ID NO:29.

The therapeutic efficacy of treatment with the BBB-transporting IDUA fusion protein may be assessed using one or more efficacy measurements that have been used for approved and experimental MPS1 therapies. Such efficacy measurements typically include: reduction in urinary glycosaminoglycan (e.g., heparan sulfate) levels; reduction in liver volume; stable forced vital capacity; increase in 6-minute walk distance; improvement in the apnea/hypopnea index; increase in shoulder flexion; improvement in the Child Health Assessment Questionnaire/Health Assessment Questionnaire disability index. In an embodiment, the efficacy measurement is taken at a desired timepoint after implant of the cells expressing the GGG-transporting IDUA fusion protein and is compared to the baseline level prior to implant. In an embodiment, the desired timepoint is any one or more of 15 days, 30 days, 60 days, 120 days, one year or longer.

In an embodiment, treatment of a subject with a BBB-transporting IDUA fusion protein described herein results in reduced heparan sulfate levels in the subject's brain and optionally in one or more non-CNS organs and tissues, e.g., liver, spleen, kidney, heart and lung. In an embodiment, heparan sulfate levels are reduced by at least 10%, 25%, 50% or more at the desired timepoint.

### ENUMERATED EXEMPLARY EMBODIMENTS

1. A fusion protein which comprises an N-terminal to C-terminal structure defined by formula I: AB-L1-RB-L2-C or by formula II: RB-L1-AB-L2-C, wherein in each of formula I and II:
AB comprises a domain that binds to human serum albumin (HSA);
L1, which may be present or absent, comprises a first linker amino acid sequence;
RB comprises a domain that binds to the extracellular domain of human IGF1R (hIGF1R);
L2, which may be present or absent, comprises a second linker amino acid sequence that is the same or different than the first linker amino acid sequence; and
C is a cargo moiety.

2. The fusion protein of embodiment 1, which is defined by formula I: AB-L1-RB-L2-C.

3. The fusion protein of embodiment 1, which is defined by formula II: RB-L1-AB-L2-C.

4. The fusion protein of any of the above embodiments, wherein AB has a molecular weight of less than about any of 75 kDa, 50 kDa or 25 kDa.

5. The fusion protein of any of the above embodiments, wherein RB has a molecular weight of less than about any of 75 kDa, 50 kDa or 25 kDa.

6. The fusion protein of any one of the above embodiments, wherein the cargo moiety has a molecular weight of about 1 kD to about 200 kD, or about 2 kD to about 100 kD.

7. The fusion protein of any one of the above embodiments, wherein the cargo moiety consists essentially of, or consists of, the amino acid sequence of a polypeptide.

8. The fusion protein of any one of the above embodiments, wherein (i) AB comprises first, second and third amino acid sequences corresponding to the three complementarity determining regions CDR1, CDR2 and CDR3 of the heavy chain variable region of an anti-HSA antibody or (ii) AB comprises the CDR1, CDR2 and CDR3 sequences of the R11 sdAb, R28 sdAb, M75 sdAb or M79 sdAb set forth in Table 2A.

9. The fusion protein of any one of the above embodiments, wherein AB comprises, consists essentially of, or consists of the amino acid sequence from a single domain antibody (sdAb), optionally wherein the amino acid sequence is selected from the group consisting of the R11, R28, M75 and M79 amino acid sequences disclosed in Table 2B above, the Alb-1 and Alb-8 amino acid sequences described in Table II and Table III of WO 2006/22787, and the Alb-23 amino acid sequences described in WO 2012/175400.

10. The fusion protein of claim 7, wherein the AB CDR1 sequence is GRTFIAYA (SEQ ID NO:1) or a conservatively substituted variant thereof; the AB CDR2 sequence is ITNFAGGTT (SEQ ID NO:2) or a conservatively substituted variant thereof; and the AB CDR3 sequence is AADRSAQTMRQVRPVLPY (SEQ ID NO:3) or a conservatively substituted variant thereof.

11. The fusion protein of any one of the above embodiments, wherein AB comprises, consists essentially of, or consists of the amino acid sequence from a sdAb.

12. The fusion protein of any one of the above embodiments, wherein AB consists essentially of, or consists of, a parental or humanized sequence shown in Table 2B above.

13. The fusion protein of any one of embodiments 1 to 12, wherein AB consists essentially of, or consists of: QVQLVESGGGLVQAGGSLRLSCVASGRTFIAYAMGWFRQAPGKEREFVAAITNF AGGTTYYADSVKGRFTISRDNAKTTVYLQMNSLKPEDTALYYCAADRSAQTMR QVRPVLPYWGQGTQVTVSS (SEQ ID NO:4), or a conservatively substituted variant thereof.

14. The fusion protein of any one of embodiments 1 to 12, wherein AB consists essentially of, or consists of: QVQLVESGGGLVQPGGSLRLSCAASGRTFIAYAMGWFRQAPGKEREFVAAITNF AGGTTYYADSVKGRFTISRDNAKTTVYLQMNSLRAEDTAVYYCAADRSAQTMR QVRPVLPYWGQGTLVTVSS (SEQ ID NO:5), or a conservatively substituted variant thereof.

15. The fusion protein of any one of embodiments 1 to 10, wherein AB consists essentially of, or consists of, an amino acid sequence of the heavy chain variable region of an antibody that cross-competes with a sdAb consisting of SEQ ID NO:4 or SEQ ID NO:5 for binding to HSA.

16. The fusion protein of any one of the above embodiments, wherein the fusion protein binds via AB to domain 1 (DI) or domain 2 (DII) of HSA and does not substantially inhibit binding of human FcRn to HSA.

17. The fusion protein of any one of the above embodiments, wherein the fusion protein binds via the AB domain to HSA with a dissociation constant (K_{D}) affinity of less than about 0.1 nM to about 1,000 nM within a pH range of about 5.0 to about 7.4 as determined by surface plasmon resonance at 25° C.

18. The fusion protein of embodiment 17, wherein the fusion protein binds via the AB domain to HSA with a K_{D} of about 0.5 nM to about 500 nM, about 1 nM to about 250 nM, about 5 nM to about 50 nM, about 10 nM to about 25 nM, or about 0.5 nM to about 1 nM within a pH range of about 5.5 to about 7.4.

19. The fusion protein of any one of the above embodiments, wherein the fusion protein binds via AB to at least one mammalian serum albumin ortholog at 25° C. within a pH range of about 5.5 to about 7.4.

20. The fusion protein of any one of the above embodiments, wherein the fusion protein binds via AB to two or more mammalian serum albumins selected from the group consisting of mouse serum albumin, rat serum albumin, hamster serum albumin, rabbit serum albumin, guinea pig albumin, pig albumin, cat albumin, dog albumin, and a non-human primate serum albumin, optionally wherein the non-human primate serum albumin is cynomolgus serum albumin or rhesus monkey serum albumin.

21. The fusion protein of any one of the above embodiments, wherein the fusion protein binds via the RB domain to hIGF1R expressed on the surface of human brain endothelial cells.

22. The fusion protein of any one of the above embodiments, wherein the fusion protein does not substantially bind to the human insulin receptor (h-IR).

23. The fusion protein of any one of the above embodiments, wherein the fusion protein does not substantially inhibit binding of insulin, insulin growth factor 1 (IGF1) or insulin growth factor 2 (IGF2) to hIGF1R.

24. The fusion protein of any one of the above embodiments, wherein the fusion protein binds via RB to an epitope in the hIGF1R extracellular domain which comprises FENFLHNSIFVPR (SEQ ID NO:6).

25. The fusion protein of any one of the above embodiments, wherein the fusion protein binds via RB to hIGF1R with a K_{D} affinity of about 0.1 nM to about 1,000 nM within a pH range of about 5.0 to about 7.4, as determined by surface plasmon resonance at 25° C.

26. The fusion protein of embodiment 24, wherein the fusion protein binds via RB to hIGF1R with a K_{D} of (i) about 0.2 nM to about any one of 500 nM, 250 nM, 100 nM, 50 nM, 25 nM or 10 nM; (ii) about 0.5 nM to about any one of 250 nM, 100 nM, 50 nM, 25 nM, 10 nM or 5 nM; or (iii) about 1 nM to about any one of 100 nM, 50 nM, 25 nM, 10 nM or 5 nM.

27. The fusion protein of embodiment 25, wherein the fusion protein binds via RB to hIGF1R with a K_{D} of 1 nM to 10 nM.

28. The fusion protein of any one of the above embodiments, wherein the fusion protein binds via RB to at least one mammalian IGF1R ortholog at 25° C. and within a pH range of about 5.0 to about 7.4.

29. The fusion protein of any one of the above embodiments, wherein the fusion protein binds via RB to two or more mammalian IGF1R proteins selected from the group consisting of mouse IGF1R, rat IGF1R, hamster IGF1R, rabbit IGF1R, guinea pig IGF1R, dog IGF1R, cat IGF1R and a non-human primate IGF1R, optionally wherein the non-human primate IGF1R is cynomolgus IGF1R or rhesus monkey IGF1R.

30. The fusion protein of any one of the above embodiments, wherein (i) RB comprises a set of first, second and third amino acid sequences corresponding to the three complementarity determining regions CDR1, CDR2 and CDR3 of the heavy chain variable region of an anti-hIGF1R antibody or (ii) RB comprises a set of first, second and third amino acid sequences selected from the CDR1, CDR2 and CDR3 amino acid sequence of the IGF1R-5 sdAb, the IGF1R-3 sdAb and the IGF1R-4 sdAb set forth in Table 3A above.

31. The fusion protein of embodiment 30, wherein the RB CDR1 sequence is GRTIDNYA (SEQ ID NO:7) or a conservatively substituted variant thereof; the RB CDR2 sequence is IDWGDGGX, where X is A or T (SEQ ID NO:8) or a conservatively substituted variant thereof; and the B3 CDR3 sequence is AMARQSRVNLDVARYDY (SEQ ID NO:9) or a conservatively substituted variant thereof.

32. The fusion protein of embodiment 31, wherein the RB CDR2 sequence is IDWGDGGA (SEQ ID NO:10).

33. The fusion protein of embodiment 30, wherein RB consists essentially of, or consists of, a parental or humanized sequence shown in Table 3B above.

34. The fusion protein of embodiment 30, wherein RB consists essentially of, or consists of: QVKLEESGGGLVQAGGSLRLSCAASGRTIDNYAMAWSRQAPGKDREFVATIDW GDGGARYANSVKGRFTISRDNAKGTMYLQMNNLEPEDTAVYSCAMARQSRVN LDVARYDYWGQGTQVTVSS (SEQ ID NO:11) or a conservatively substituted variant thereof.

35. The fusion protein of embodiment 30, wherein RB consists essentially of, or consists of: QVQLVESGGGLVQPGGSLRLSCAASGRTIDNYAMAWVRQAPGKGLEWVATID WGDGGTRYANSVKGRFTISRDNSKNTMYLQMNSLRAEDTAVYYCAMARQSRV NLDVARYDYWGQGTLVTVSS (SEQ ID NO:12) or a conservatively substituted variant thereof.

36. The fusion protein of any one of embodiments 1 to 30, wherein RB consists essentially of, or consists of, an amino acid sequence of the heavy chain variable region of an antibody that (i) cross-competes with a sdAb consisting of SEQ ID NO:11 or SEQ ID NO:12 for binding to hIGF1R or (ii) cross-competes with any of the 996, 1226 and 1564 antibodies described in EP3725806A1.

37. The fusion protein of any one of the above embodiments, wherein L1 and L2 are present.

38. The fusion protein of embodiment 37, wherein each of L1 and L2 is a linker peptide that is less than 50 amino acids in length, optionally wherein the length of each of L1 and L2 is between about 15 and 30 amino acids, or between about 20 and 25 amino acids.

39. The fusion protein of embodiment 38, wherein L1 consists essentially of, or consists of, (GGGGS)m, wherein m is 4 or 5.

40. The fusion protein of any one of embodiments 37 to 39, wherein L2 consists essentially of, or consists of, (GGGGS)n, wherein n is 4 or 5.

41. The fusion protein of embodiment 39, wherein each of L1 and L2 consists essentially of, or consists of: GGGGSGGGGSGGGGSGGGGS (SEQ ID NO:14).

42. The fusion protein of any one of the above embodiments, wherein the cargo moiety is a cargo polypeptide selected from the group consisting of: an enzyme, a growth factor, a cytokine, or an antibody or antigen binding fragment thereof.

43. The fusion protein of embodiment 42, wherein the cargo polypeptide comprises the mature amino acid sequence encoded by a wild-type human gene.

44. The fusion protein of embodiment 42 or 43, wherein the cargo polypeptide is an enzyme.

45. The fusion protein of any one of embodiments 42 to 44 wherein the cargo polypeptide is an acid alpha-glucosidase protein (GAA), an alpha-galactosidase A (GLA) protein, an alpha-L-iduronidase (IDUA) protein, an alpha-N-acetyl-glucosaminidase (NAGLU) protein, a beta-glucoronidase (GUSB) protein, a beta-glucosidase (GBA) protein, an iduronate-2-sulfatase (IDS) protein, an heparan-alpha-glucosaminide N-acetyltransferase (HGSNAT) protein, an N-acetylgalactosamine-6-sulfatase (GNS) protein, or an N-sulfoglucosamine sulfohydrolase (SGSH) protein, optionally wherein the cargo polypeptide is not an IDS protein.

46. The fusion protein of embodiment 45, wherein the cargo polypeptide is an IDUA protein, optionally wherein the cargo polypeptide comprises, consists essentially of, or consists of the mature amino acid sequence encoded by the wild-type human IDUA gene.

47. The fusion protein of any one of embodiments 42 to 46, wherein the cargo polypeptide is an IDUA protein and comprises amino acid 26, 27 or 28 to amino acid 653 of the precursor human IDUA amino acid sequence shown in FIG. 1H.

48. The fusion protein of any one of embodiments 42 to 47, wherein the cargo polypeptide is an IDUA protein and consists essentially of amino acids 27-653 of the amino acid sequence shown in FIG. 1H.

49. The fusion protein of any one of embodiments 46 to 48, which has an IDUA enzymatic activity that is within 80-120% of the corresponding enzymatic activity of wild-type human IDUA protein.

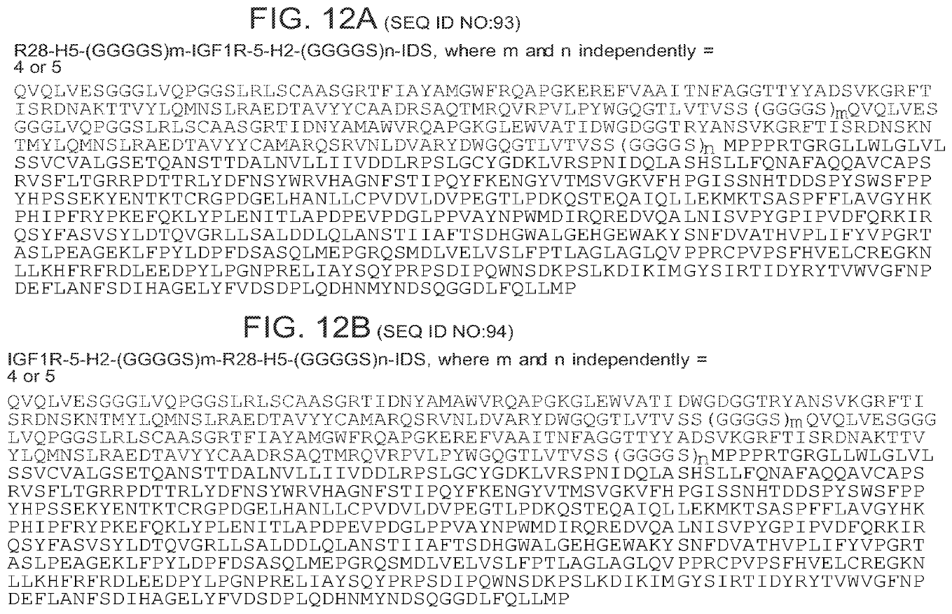

50. The fusion protein of any one of embodiments 42 to 45, wherein the cargo polypeptide is an IDS protein, optionally where the cargo polypeptide comprises, consists essentially of, or consists of the amino acid sequence shown in FIG. 12A or FIG. 12B.

51. The fusion protein of embodiment 50, which has an IDS enzymatic activity that is within 80-120% of the corresponding enzymatic activity of wild-type human IDS protein.

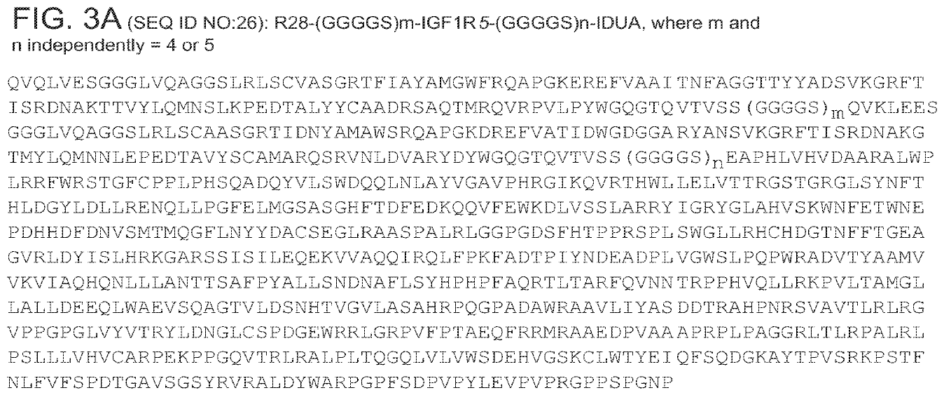

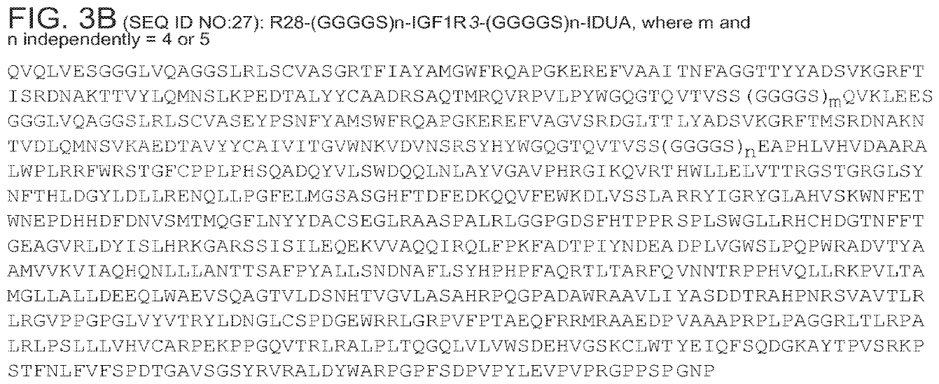

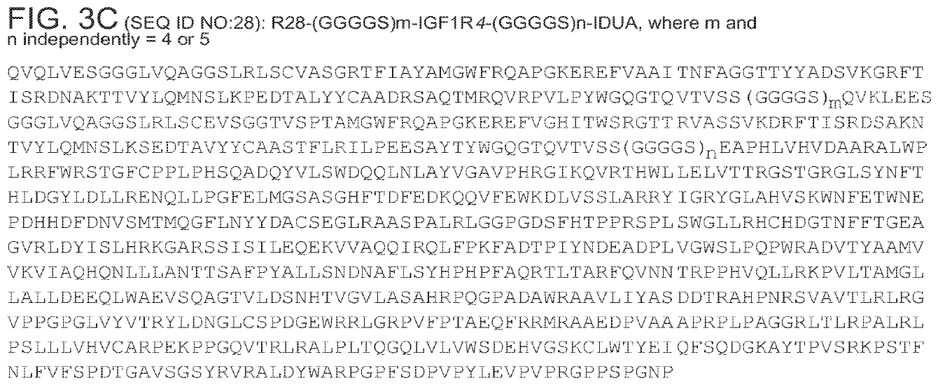

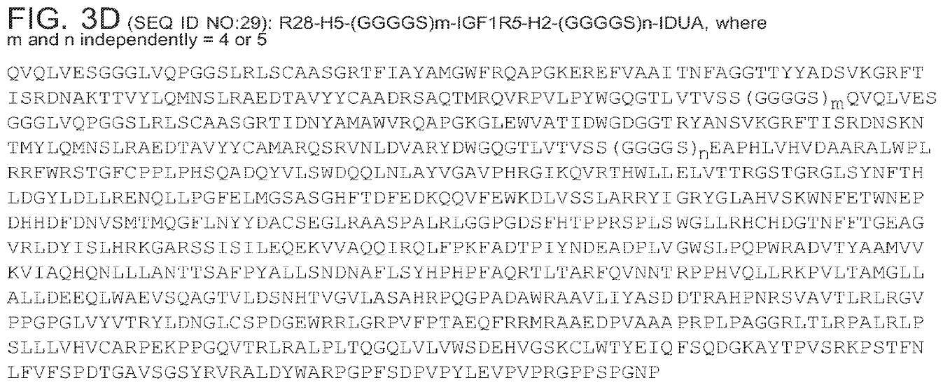

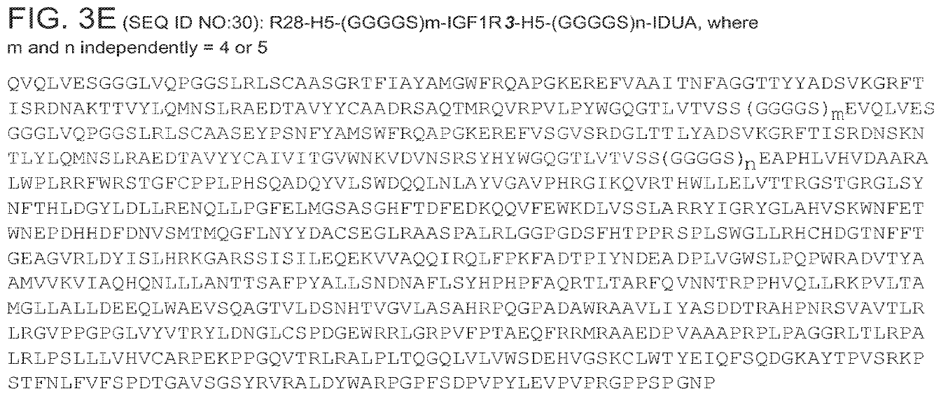

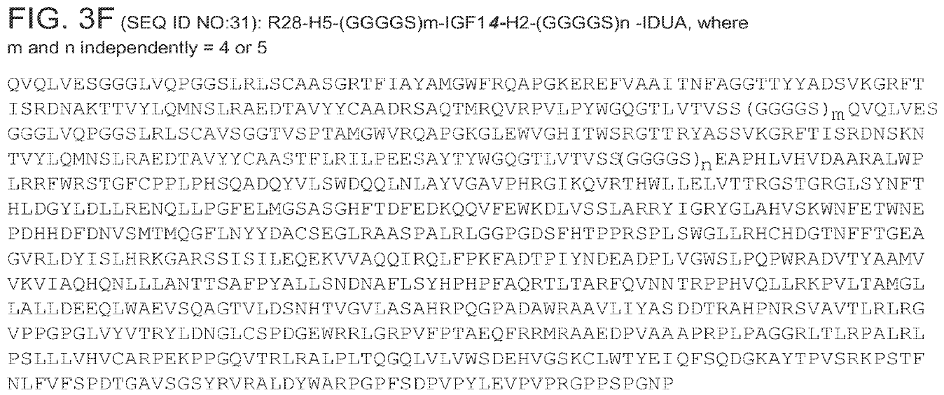

52. The fusion protein of embodiment 1, which comprises, consists essentially of, or consists of the amino acid sequence of an hIDUA fusion protein shown in FIG. 3A, FIG. 3B, FIG. 3C, FIG. 3D, FIG. 3E or FIG. 3F, optionally wherein the fusion protein consists essentially of, or consists of the amino acid sequence shown in SEQ ID NO:29, wherein each of m and n=4.

53. The fusion protein of embodiment 1, which comprises formula I: AB-L1-RB-L2-C, wherein AB comprises, consists essentially of, or consists of SEQ ID NO:4 or SEQ ID NO:5, L1 comprises, consists essentially of, or consists of (GGGGS)n, wherein n is 4 or 5, RB comprises SEQ ID NO:11 or SEQ ID NO:12, L1 comprises, consists essentially of, or consists of (GGGGS)n, wherein n is 4 or 5, C comprises, consists essentially of, or consists of amino acids 27-653 of SEQ ID NO:22, and L2 comprises, consists essentially of, or consists of (GGGGS)n, wherein n is 4 or 5.

54. The fusion protein of embodiment 1, which comprises formula II: RB-L1-AB-L2-C, wherein AB comprises, consists essentially of, or consists of SEQ ID NO:4 or SEQ ID NO:5, L1 comprises, consists essentially of, or consists of (GGGGS)n, wherein n is 4 or 5, RB comprises SEQ ID NO:11 or SEQ ID NO:12, L1 comprises, consists essentially of, or consists of (GGGGS)n, wherein n is 4 or 5, comprises, consists essentially of, or consists of amino acids 27-653 of SEQ ID NO:22, and L2 comprises, consists essentially of, or consists of (GGGGS)n, wherein n is 4 or 5.

55. The fusion protein of embodiment 53 or 54, wherein AB consists essentially of SEQ ID NO:5 and RB consists essentially of SEQ ID NO:12.

56. A polynucleotide which comprises a first nucleotide sequence that encodes the fusion protein of any one of the above embodiments.

57. The polynucleotide of embodiment 56, wherein the first nucleotide sequence is operably linked to a nucleotide sequence encoding a secretory signal sequence for the fusion protein, optionally wherein the secretory signal sequence consists essentially of, or consists of, (i) MELGLSWVVLAALLQGVQA (SEQ ID NO:79) or (ii) one of the amino acid sequences set forth in Table 5.

58. The polynucleotide of embodiment 57, wherein the secretory signal sequence consists essentially of, or consists of MELGLSWVVLAALLQGVQA (SEQ ID NO:79).

59. The polynucleotide of any one of embodiments 56 to 58, wherein the first nucleotide sequence is operably linked to a promoter sequence and a polyA signal sequence.

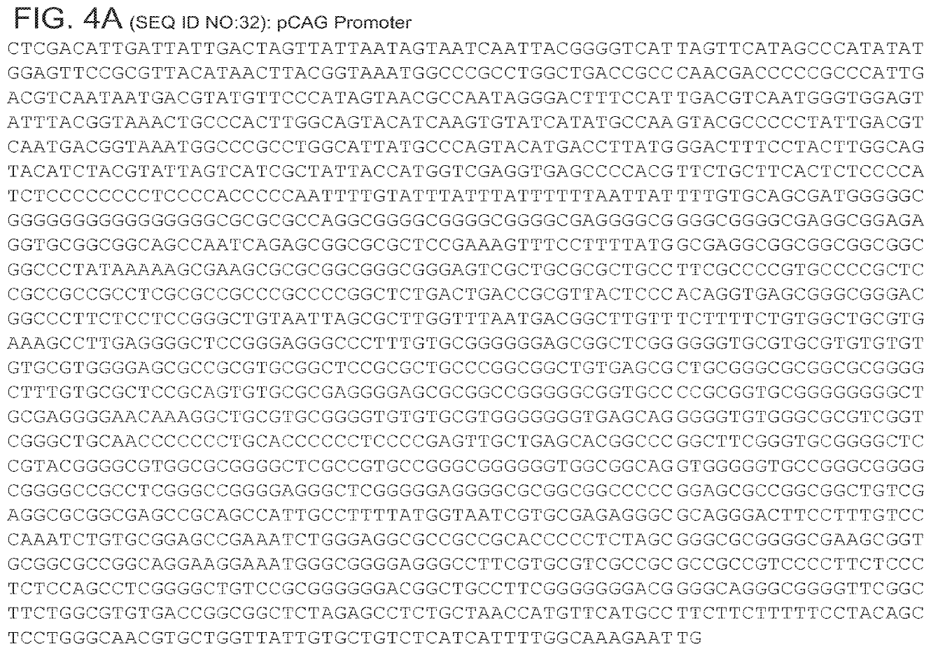

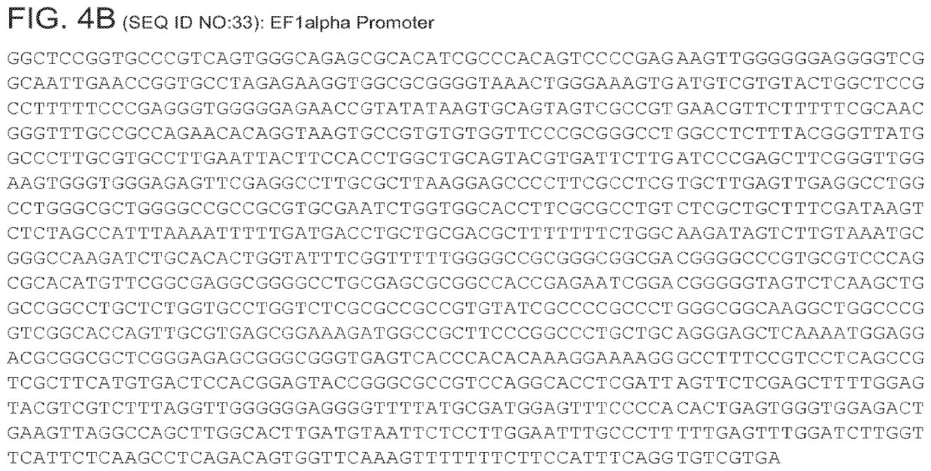

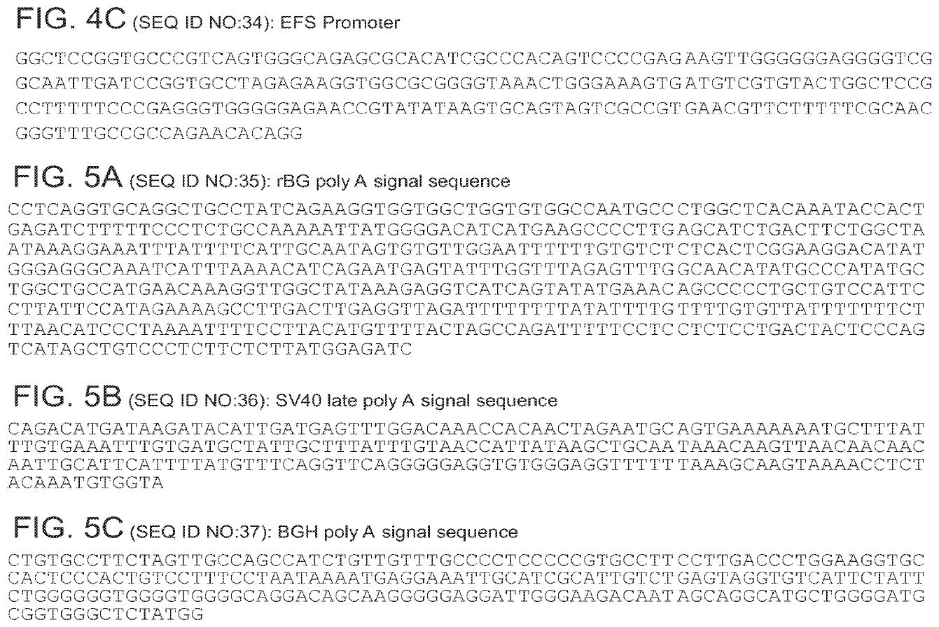

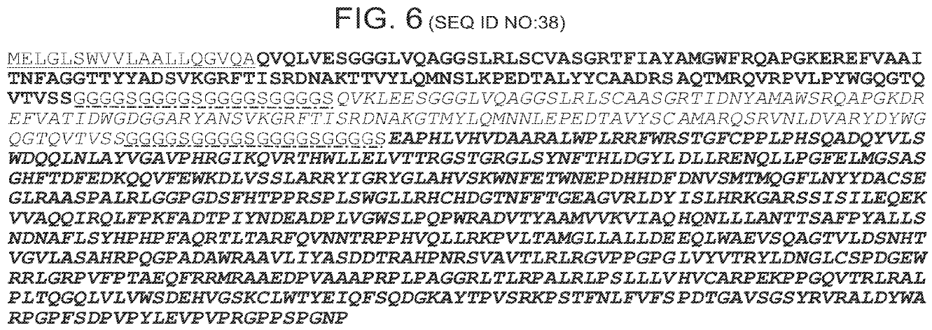

60. The polynucleotide of embodiment 59, wherein the promoter sequence is the pCAG promoter sequence shown in FIG. 4A, the EF1α promoter sequence shown in FIG. 4B or the EFS promoter sequence shown in FIG. 4C.

61. The polynucleotide of embodiment 60, wherein the promoter sequence is the EF1a promoter sequence shown in FIG. 4B.

62. The polynucleotide of any one of embodiments 59 to 61, wherein the polyA signal sequence is the rBG poly A signal sequence shown in FIG. 5A, the SV40 late poly A signal sequence shown in FIG. 5B or the BGH poly A signal sequence shown in FIG. 5C.

63. The polynucleotide of embodiment 62, wherein the polyA signal sequence is the rBG poly A signal sequence shown in FIG. 5A.

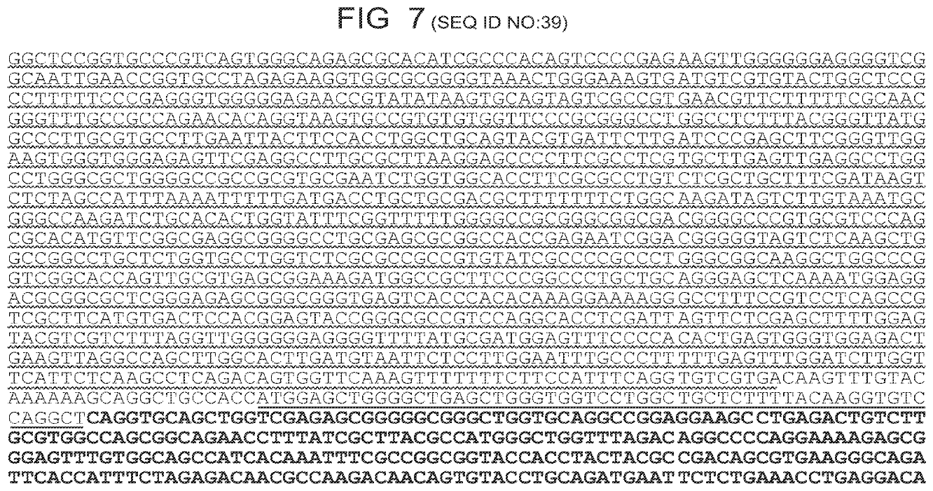

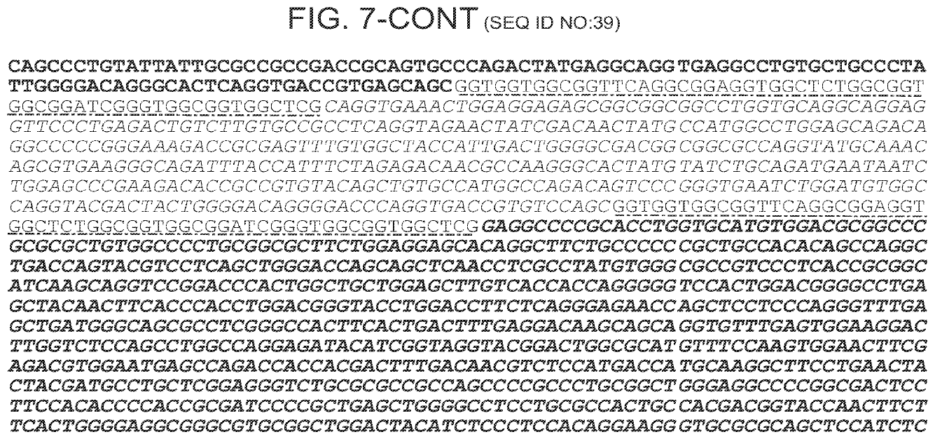

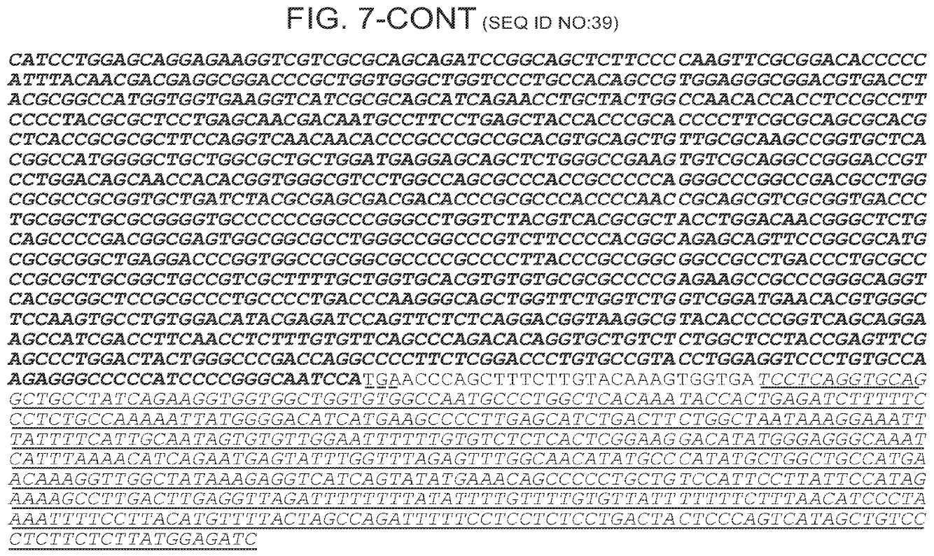

64. The polynucleotide of embodiment 56, which comprises the nucleotide sequence shown in FIG. 7.

65. The polynucleotide of any one of embodiments 56 to 64 which is one strand in an isolated double-stranded DNA molecule.

66. A genetically modified mammalian cell which is transiently or stably transfected with the polynucleotide of any one of embodiments 56 to 65.

67. The genetically modified mammalian cell of embodiment 66, wherein the polynucleotide is inserted into at least one location in the genome of the mammalian cell.

68. The genetically modified mammalian cell of embodiment 66 or 67, wherein the cell is derived from a human cell.

69. The genetically modified mammalian cell of embodiment 68 which is derived from an RPE cell, optionally an ARPE-19 cell.

70. The genetically modified mammalian cell of embodiment 68, which is derived from an induced pluripotent stem cell (iPSC) or a mesenchymal stem cell.

71. A composition comprising a plurality of genetically modified cells, wherein each cell in the plurality is a genetically modified cell as defined by any one of embodiments 68 to 70.

72. The composition of embodiment 71, wherein the plurality of genetically modified cells is obtained from a culture of a monoclonal cell line.

73. An implantable device comprising at least one cell-containing compartment which comprises the genetically modified cell of any one of embodiments 66 to 70 or the composition of embodiment 71 or 72 and further comprises at least one means for mitigating the foreign body response (FBR) when the device is implanted into the subject.

74. The implantable device of embodiment 74=3, wherein the cell-containing compartment comprises a polymer composition, wherein the polymer composition comprises an alginate covalently modified with a peptide, wherein the peptide consists essentially of or consists of GRGDSP (SEQ ID NO:89), GGRGDSP (SEQ ID NO:90) or GGGRGDSP (SEQ ID NO:91).

75. The implantable device of embodiment 73 or 74, wherein the cell-containing compartment is surrounded by a barrier compartment comprising an alginate hydrogel and optionally a compound of Formula (III) disposed on the outer surface of the barrier compartment.

76. The implantable device of embodiment 74 or 75, wherein the polymer composition comprises an alginate covalently modified with a peptide, wherein the peptide consists essentially of or consists of GRGDSP, and wherein the barrier compartment comprises an alginate chemically modified with

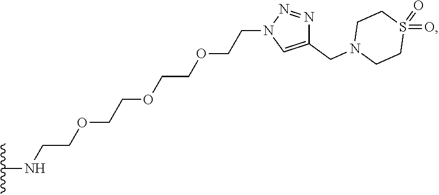 [SMILES: *NCCOCCOCCOCCn1cc(CN2CCS(=O)(=O)CC2)nn1]

or a pharmaceutically acceptable salt thereof.

77. The implantable device of any one of embodiments 73 to 76, which is a spherical, two-compartment hydrogel capsule of about 0.75 mm to about 2 mm in diameter.

78. A preparation of devices, wherein each device in the preparation is a device of any one of embodiments 73 to 77.

79. A hydrogel capsule comprising:
(a) an inner compartment which comprises a plurality of the genetically modified cell of any one of embodiments 66 to 70 encapsulated in a first polymer composition, wherein the first polymer composition comprises a hydrogel-forming polymer; and
(b) a barrier compartment surrounding the inner compartment and comprising a second polymer composition, wherein the second polymer composition comprises an alginate covalently modified with at least one compound of Formula (III) or a pharmaceutically acceptable salt thereof.

80. The hydrogel capsule of embodiment 79, wherein the compound of Formula (III) is selected from a compound provided in the table below:

| Compound No. | Structure |
|---|---|
| 100 | 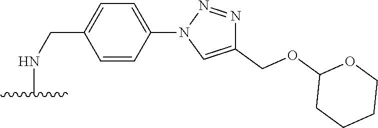 [SMILES: *NCc1ccc(-n2cc(COC3CCCCO3)nn2)cc1] |
| 101 | 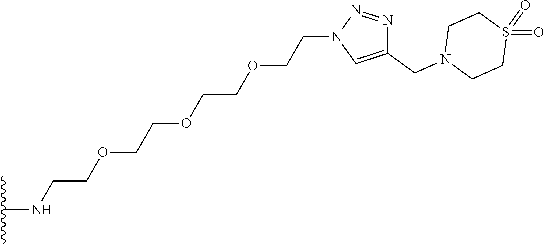 [SMILES: *NCCOCCOCCOCCn1cc(CN2CCS(=O)(=O)CC2)nn1] |
| 110 | 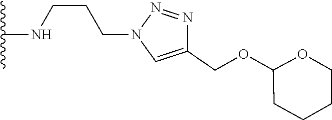 [SMILES: *NCCCn1cc(COC2CCCCO2)nn1] |
| 112 | 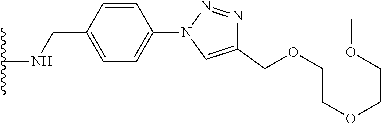 [SMILES: *NCc1ccc(-n2cc(COCCOCCOC)nn2)cc1] |
| 113 | 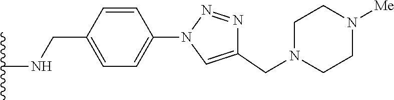 [SMILES: *NCc1ccc(-n2cc(CN3CCN(C)CC3)nn2)cc1] |
| 114 | 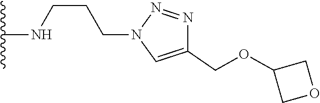 [SMILES: *NCCCn1cc(COC2COC2)nn1] |

81. The hydrogel capsule of embodiment 75 or 76, wherein the selected compound is

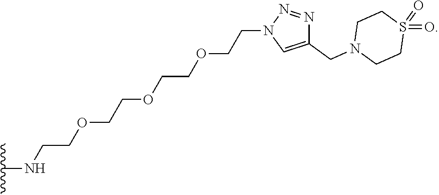 [SMILES: *NCCOCCOCCOCCn1cc(CN2CCS(=O)(=O)CC2)nn1]

82. The hydrogel capsule of any one of embodiments 79-81, wherein the concentration of the genetically modified cell in the inner compartment is at least 40 million cells per ml of the first polymer composition.

83. A capsule composition comprising a plurality of the hydrogel capsule of any one of embodiments 79 to 82 in a pharmaceutically acceptable carrier.

84. The capsule composition of embodiment 83, wherein the genetically modified cells in the plurality of the hydrogel capsule express and secrete the IDUA fusion protein of embodiment 51.

85. A pharmaceutical composition comprising the fusion protein of any one of embodiments 1 to 55 and a pharmaceutically acceptable carrier.

86. The pharmaceutical composition of embodiment 81, wherein the fusion protein is the IDUA fusion protein of embodiment 48.

87. A method of preventing or treating a disease or condition in the central nervous system (CNS) of a subject, which comprises:
(i) administering to the subject the pharmaceutical composition of embodiment 85 or 86;
(ii) implanting in the subject the device or device preparation of any one of embodiments 73 to 78; or
(iii)implanting in the subject the capsule composition of embodiment 83 or 84.

88. A method of treating a human subject diagnosed with Mucopolysaccharidosis type 1 (MPS-1) disease, comprising:
(a) providing the capsule composition of embodiment 86; and
(b) disposing the capsule composition in the body of the subject.

89. The method of embodiment 88, wherein the disposing step comprises placing the capsule composition into the intraperitoneal space of the subject.

90. The method of embodiment 88, wherein the disposing step comprises placing the capsule composition into the greater sac of the peritoneal cavity.

91. The method of any one of embodiments 87 to 90, wherein the capsule composition produces a BBB-transporting IDUA fusion protein which comprises, consists essentially or consists of SEQ ID NO:28 or SEQ ID NO:29.

### EXAMPLES

In order that the disclosure described herein may be more fully understood, the following examples are set forth. The examples described in this application are offered to illustrate the BBB-transporting fusion proteins, genetically modified cells, implantable devices, and compositions and methods provided herein and are not to be construed in any way as limiting their scope. The MPS-1 mice used in these Examples were obtained from Charles River Laboratory (Wilmington, MA) and carry a nonsense mutation at Idua codon W392 (Jackson Laboratory Stock No. 017681).

### Example 1: Effect of Fusing an Exemplary IGF1R Binding Domain to hIDUA on In Vitro hIDUA Activity

DNA expression vectors were engineered to encode six different hIDUA fusion enzymes containing a BBB-penetrant sdAb (IGF1R4) fused to the N-terminus or C-terminus of the hIDUA open reading frame via a variable length G4S linker unit repeated 3, 4 or 5 times. ARPE-19 cells were transfected with these expression vectors and polyclonal colonies stably expressing a hIDUA fusion enzyme were generated for each of the six different transfections. The expression vectors and transfected cells were named based upon the orientation of the encoding sequences for the composite modules with respect to one another. For example, hIDUA-(G4S)₃-IGF1R4 cells express a fusion enzyme in which IGF1R4 is fused to the C-terminus of hIDUA via a G4S linker repeated 3 times.

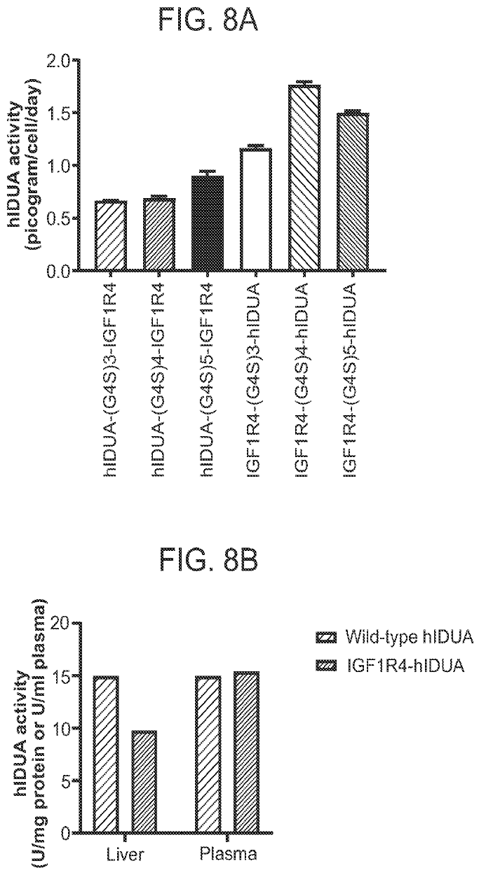

The in vitro IDUA activity of the hIDUA fusion proteins secreted from the six polyclonal colonies was assessed by seeding cells from each polyclonal colony at approximately 400,000 cells in 2 ml fresh medium per well of a 6-well tissue culture plate. Twenty to twenty-four hours later, the conditioned cell culture medium was collected and assayed for IDUA protein concentration using an IDUA activity assay, and compared to a known standard (laronidase), substantially as described in Ou, L., et al., (2014). Standardization of α-L-iduronidase enzyme assay with Michaelis-Menten kinetics. Molecular Genetics and Metabolism, 111(2), 113-115. In brief, 8 ul of the cell culture medium was added to 32 ul of 0.4M sodium formate, pH 3.5, in a black 96-well plate. 20 ul of 700 μm 4-Methylumbellifery-α-L-Iduronide diluted in assay buffer was added to all wells. The plate was incubated at 37° C. for 10 minutes. To stop the reaction, 100 ul of stop solution was added (0.5M NaOH+0.5M glycine). α-L-iduronidase catalyzed the cleavage of the non-fluorescent substrate (4MU-iduronide) into a fluorescent product (4-MU). Fluorescence intensity was measured on a Biotek Cytation 3 at excitation and emission wavelengths of 365 nm and 445 nm (top read), respectively, in endpoint mode. Enzyme activity levels were compared to a standard curve generated with laronidase and the results are shown in FIG. 8. The polyclonal culture that produced the highest in vitro IDUA activity was the one expressing the IGF1R4-(G4S)₄-hIDUA fusion enzyme.

The expression vector for this fusion was used to perform a new transfection of ARPE-19 cells, from which a higher hIDUA activity polyclonal pool was identified. Cells from this pool were encapsulated at 50 million cell/ml in the inner compartment of shielded capsules, essentially as described in WO2020198696. A dose of 0.5 ml capsules was implanted in the IP space of each of five MPS-1 mice. At 28 days post-administration, the mice were euthanized, and the amount of hIDUA activity in liver and plasma tissue samples was assessed. FIG. 8B shows the measured hIDUA activity levels compared with typical hIDUA levels observed in substantially similar experiments with implanted capsules encapsulating ARPE-19 cells genetically modified to express and secrete wild-type hIDUA. This comparison indicates that implanting MPS-1 mice with cells producing the IGF1R4-hIDUA fusion did not result in higher liver and plasma hIDUA activity compared to implanting cells producing wild-type hIDUA.

### Example 2: Effect of Fusing Exemplary HSA and IGF1R Binding Domains to hIDUA on In Vitro hIDUA Activity

DNA expression vectors were engineered to encode six different hIDUA fusion enzymes containing different orientations of open reading frames for a BBB-penetrant sdAb (IGF1R5), an anti-HSA sdAb (R28) and hIDUA with a (G4S)₄ amino acid linker located between each of the open reading frames. The different orientations evaluated were: (i) 2 fusions in which the sdAbs were both fused to the N-terminus of hIDUA (N-terminal fusions); (ii) 2 fusions in which the sdAbs flanked hIDUA (flanking fusions); and (iii) 2 fusions in which the sdAbs were both fused to the C-terminus of hIDUA (C-terminal fusions). ARPE-19 cells were transfected with these expression vectors and polyclonal colonies stably expressing a hIDUA fusion enzyme were generated for each of the six different transfections. The expression vectors and transfected cells were named based upon the orientation of the encoding sequences for the composite modules with respect to one another. For example, IGF1R5-R28-hIDUA cells express a fusion enzyme in which IGF1R5 is N-terminal to R28, which is itself N-terminal to hIDUA.

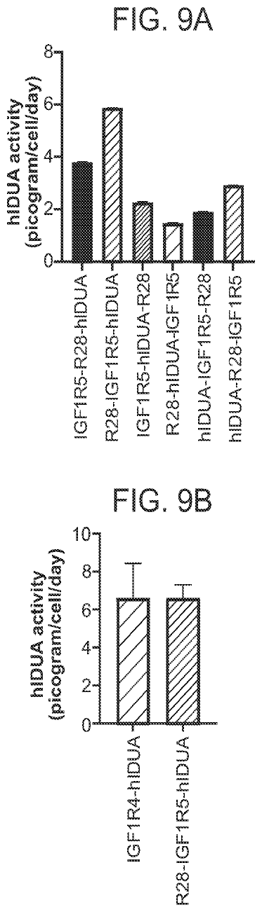

The in vitro hIDUA activity of the hIDUA fusion proteins secreted from the six polyclonal colonies was assessed as described in Example 1, and the results are shown in FIG. 9A. The polyclonal pools expressing the N-terminal double fusion enzymes (IGF1R5-R28-hIDUA and R28-IGF1R5-hIDUA) exhibited higher hIDUA activity levels than the cell lines expressing the flanking fusions or the C-terminal fusions, results that are consistent with the hIDUA activity results obtained in Example 1, which showed higher hIDUA activity generated for the single N-terminal fusions than the C-terminal fusions. Surprisingly, the orientation of the anti-HSA and anti-IGF1R sdAbs in the N-terminal fusions had a significant effect on hIDUA activity, with the fusion containing R28 upstream of IGF1R5 generating about 50% higher hIDUA activity than the fusion with IGF1R-5 upstream of R28. The difference in activity between each of these six cell lines is statistically significant.

To assess whether the addition of the HSA binding domain affected hIDUA activity of the fusion protein, the in vitro hIDUA activity in conditioned media from culturing cells from the top-performing double fusion polyclonal pool (R28-(G4S)₄-IGF1R5-(G4S)₄-hIDUA) was compared to the in vitro hIDUA activity in conditioned media from culturing cells from the IGF1R4-(G4S)₄-hIDUA polyclonal pool used for the MPS-1 mice experiment described in Example 1. As shown in FIG. 9B, there was essentially no difference in the amount of hIDUA activity in the single N-terminal and double N-terminal fusion cultures.

### Example 3: Effect of HSA and IGF1R Binding Domains on In Vivo hIDUA Activity

Cells from the polyclonal pool secreting the R28-(G4S)₄-IGF1R5-(G4S)₄-hIDUA fusion enzyme were encapsulated at 50 million cell/ml in the inner compartment of shielded capsules, essentially as described in WO2020198696. A dose of 0.5 ml capsules was implanted in the IP space of each of six MPS-1 mice (J). At 21 days post-administration, the mice were euthanized, and the amount of hIDUA activity in plasma and various systemic tissues was assessed.

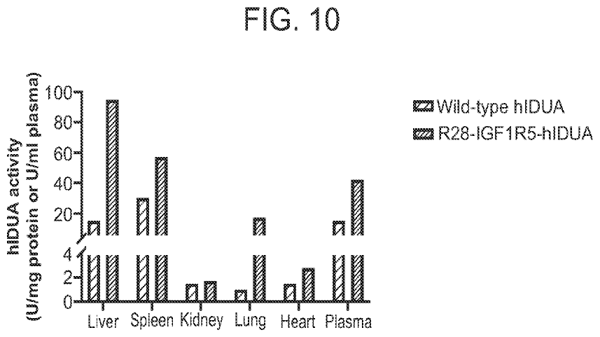

FIG. 10 shows the measured hIDUA activity levels compared with typical hIDUA levels observed in substantially similar experiments with implanted shielded capsules encapsulating ARPE-19 cells genetically modified to express and secrete wild-type hIDUA. Mice implanted with cells expressing the R28-IGF1R5-hIDUA fusion enzyme had substantially higher hIDUA activity in tissues (except for kidney) and plasma than mice implanted with cells expressing the wild-type hIDUA enzyme.

### Example 4: Effect of Implanting MPS-1 Mice with Shielded Capsules Producing an Exemplary BBB-Transporting Fusion Protein on Brain Heparan Sulfate Levels

Shielded capsules encapsulating cells from the polyclonal pool secreting the R28-(G4S)₄. IGF1R5-(G4S)₄-hIDUA fusion enzyme were implanted into the IP space of each of six MPS-1 mice. A group of six, untreated MPS-1 mice were observed as a control. At 21 days post-administration, both groups of mice were euthanized, and the amount of heparan sulfate in two distinct brain regions (hippocampus and frontal lobe) was assessed. In brief, tissue homogenates of each brain region were obtained, and tissue homogenates (10 ul) were combined with an equal volume of a heparinase cocktail (a mixture of heparinase I, heparinase II and heparinase III in a heparinase reaction buffer). The individual heparinases and heparinase reaction buffer were from New England Biolabs, catalogue numbers P0735S, P0736S, P0737S and B0735S). The reaction was incubated at 37° C. for 3 days, and the resulting heparan sulfate disaccharides were quantified via LC/MS.

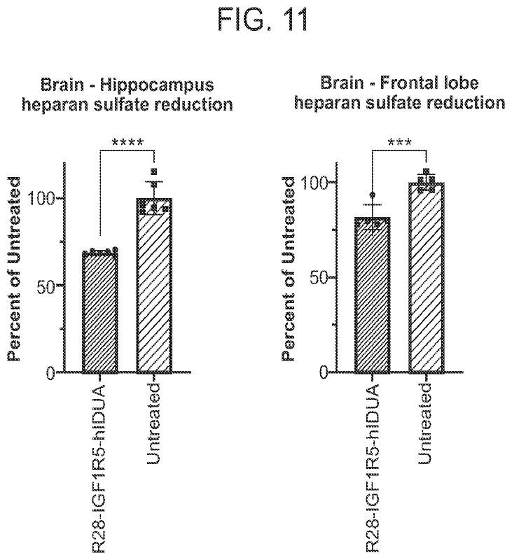

As shown in FIG. 11, MPS-1 mice implanted with cells secreting the hIDUA BBB-transporting fusion protein had a statistically significant reduction in heparan sulfate in both brain tissues compared to the control mice.

### Example 5: Double IDS Fusions with Different Orientations of Exemplary HSA and IGF1R Binding Domains

DNA expression vectors were engineered to encode two different hIDS fusion enzymes containing different orientations of open reading frames for a BBB-penetrant sdAb (IGF1R5), an anti-HSA sdAb (R28) and hIDS with a (G4S)₄ amino acid linker located between each of the open reading frames. The different orientations evaluated were R28-(G4S)₄-IGF1R5-(G4S)₄-hIDS (e.g., a formula I fusion) and IGF1R5-(G4S)₄-R28-(G4S)₄-hIDS (e.g., a formula II fusion). ARPE-19 cells were transfected with one of the two expression vectors and polyclonal colonies stably expressing a hIDS fusion enzyme were generated for each of the two transfections.

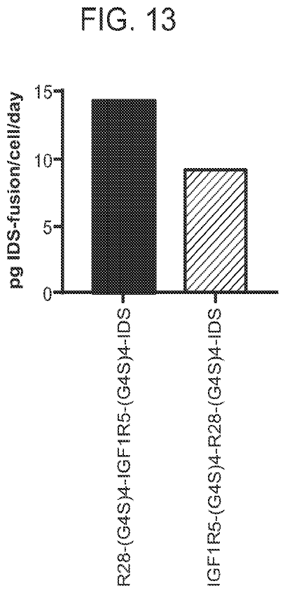

The in vitro IDS activity of the hIDS fusion proteins secreted from each polyclonal pool was assessed by seeding cells from each cell line at approximately 400,000 cells in 2 ml fresh medium per well of a 6-well tissue culture plate. Twenty to twenty-four hours later, the conditioned cell culture medium was collected and assayed for IDS protein concentration using a 2-step enzymatic IDS activity assay and compared to a known standard (idursulfase). In brief, 3 ul of the cell culture medium was added to 15.6 ul of water and 20 ul buffer containing 20 mM lead(II) acetate in 0.2 M sodium acetate (pH 5.0), in a black 96-well plate. 1.4 ul of 20 mM substrate (4-Methylumbelliferyl-a-L-Iduronide 2-sulfate reconstituted in DMSO) was added to all wells. The plate was incubated at 37° C. for 10 minutes. To quench the first reaction, 40 ul of 2× McIlvaine's buffer (0.40 M sodium phosphate, 0.20 M citrate pH 4.5) supplemented with 5 ug/mL of laronidase was added to each well. The plate was incubated at 37° C. for 10 minutes. To stop the second reaction, 100 ul of stop solution was added (0.5M NaOH+0.5M glycine). In the first reaction step, the substrate (4MU-α-iduronide 2-sulfate) is hydrolyzed by Iduronate 2-Sulfatase (IDS) to product 4MU-iduronide. In the second reaction step, Iduronate 2-Sulfatase activity is quenched by addition of excess phosphate, and α-L-iduronidase catalyzed the cleavage of the non-fluorescent product of the first reaction (4MU-iduronide) into a fluorescent product (4-MU). Fluorescence intensity was measured on a Biotek Cytation 3 at excitation and emission wavelengths of 365 nm and 445 nm (top read), respectively, in endpoint mode. Enzyme activity levels were compared to a standard curve generated with idursulfase, and the results are shown in FIG. 13. The polyclonal pools expressing the R28-(G4S)₄—IGF1R5-(G4S)₄-hIDS (formula I) double fusion enzyme exhibited higher hIDS activity levels than the cell lines expressing the IGF1R5-(G4S)₄-R28-(G4S)₄-hIDS (formula II) double fusion, results that are consistent with the hIDUA activity results obtained in Example 3, which showed higher hIDUA activity generated for the formula I fusion (AB-L1-RB-L2-C) than the formula II fusion (RB-L1-AB-L2-C).

### Example 6: Effect of Implanting MPS-1 Mice with Shielded Capsules Producing an Exemplary BBB-Transporting Fusion Protein on Heparan Sulfate in Systemic Tissues

Shielded capsules encapsulating cells from the polyclonal pool secreting the R28-(G4S)₄. IGF1R5-(G4S)₄-hIDUA fusion enzyme were implanted into the IP space of each of six MPS-1 mice. A group of six, untreated MPS-1 mice were observed as a control. At 21 days post-administration, both groups of mice were euthanized, and the amount of heparan sulfate in various organs (liver, spleen, kidney, lung and heart) was assessed. In brief, organ tissue homogenates were obtained, and the tissue homogenates (10 ul) were combined with an equal volume of a heparinase cocktail (a mixture of heparinase I, heparinase II and heparinase III in a heparinase reaction buffer). The individual heparinases and heparinase reaction buffer were from New England Biolabs, catalogue numbers P0735S, P0736S, P0737S and B0735S. The reaction was incubated at 37° C. for 3 days, and the resulting heparan sulfate disaccharides were quantified via LC/MS.

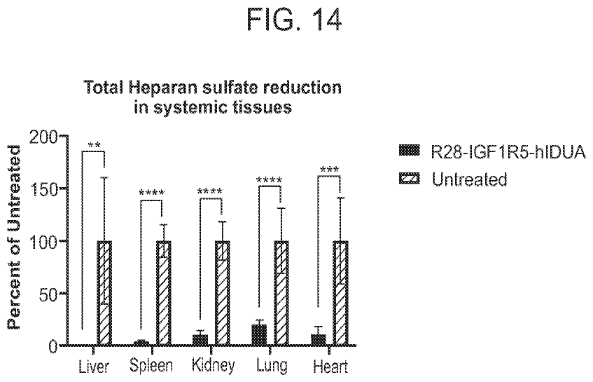

As shown in FIG. 14, MPS-1 mice treated with the dual fusion enzyme (black bars) exhibited statistically significant reduction in heparan sulfate levels relative to untreated mice (grey bars) in each of the liver, spleen, kidney, lung and heart tissue samples.

### Example 7: Assessment of Immunogenicity Potential of an Exemplary BBB-Transporting Fusion Protein

A commercial vendor (EpiVax, Providence, Rhode Island, USA) performed in silico immunogenicity assessments on the following molecules: R28-(G4S)₄-IGF1R5-(G4S)₄-hIDUA fusion enzyme; (2) R28-H5-(G4S)₄—IGF1R5-H2-(G4S)₄-hIDUA fusion enzyme, in which the R28-H5 component is a humanized R28 variant with the amino acid sequence (SEQ ID NO:5), and the IGF1R5-H2 component is a humanized IGF1R5 variant with the amino acid of (SEQ ID NO:12); (3) native human IDUA protein alone (i.e., not fused to any other molecule); and (4) the individual parental and humanized sdAbs present in the two fusion enzymes. In brief, EpiVax evaluated the amino acid sequences of the fusion constructs and of the individual comprising molecules for predicted T-cell epitopes using their proprietary EpiMatrix algorithm. Each molecule was scored for total predicted T-cell epitope content and ranked against the EpiMatrix Protein Immunogenicity Scale where the molecules could be compared directly to proteins of known immunogenicity. As shown in the table immediately below, each of the fusion enzymes had a lower predicted immunogenicity score than native hIDUA alone.

| | Immunogenicity | Immunogenicity |
|---|---|---|
| Molecule | Score | Risk Level |
| R28 | 15.19 | Elevated |
| hIDUA alone | 6.19 | Neutral |
| R28-(G4S)₄₋IGF1R5- | −5.20 | Neutral |
| (G4S)₄₋hIDUA | | |
| R28-H5-(G4S)₄₋IGF1R5- | −14.08 | Neutral |
| H2-(G4S)₄-hIDUA | | |
| IGF1R5 | −22.32 | Low |
| R28-H5 | −32.63 | Low |
| IGF1R5-H2 | −46.54 | Low |

### Example 8: Comparison of Exemplary Humanized and Camelid BBB-Transporting Fusion Proteins In Vivo

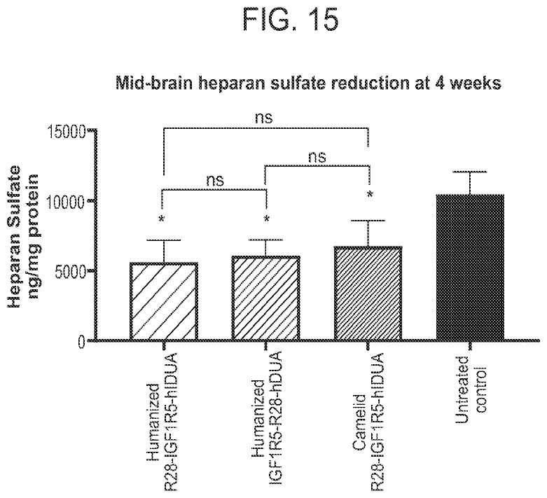

Three groups of MPS-1 mice (six mice per group) were implanted with shielded capsules encapsulating cells from polyclonal pools secreting a humanized IDUA fusion protein [R28-H5-(G4S)₄-IGF1R5-H2-(G4S)₄-hIDUA or IGF1R5-H2-(G4S)₄-R28-H5-(G4S)₄-hIDUA] or a camelid IDUA fusion protein [R28-(G4S)₄—IGF1R5-(G4S)₄-hIDUA]. The IDUA amino acid sequence in each fusion protein was identical. A group of four, untreated MPS-1 mice were observed as a control. At 28 days post-administration, all mouse cohorts were euthanized, and the amount of heparan sulfate in the mid-brain was assessed. In brief, mid-brain tissue homogenates were obtained, and tissue homogenates (10 ul) were combined with an equal volume of a heparinase cocktail (a mixture of heparinase I, heparinase II and heparinase III in a heparinase reaction buffer. The individual heparinases and heparinase reaction buffer were from New England Biolabs, catalogue numbers P0735S, P0736S, P0737S and B0735S. The reaction was incubated at 37° C. for 3 days, and the resulting heparan sulfate disaccharides were quantified via LC/MS. As shown in FIG. 15, MPS-1 mice treated with any of the three dual fusion enzymes (grey bars) exhibited statistically significant reduction in heparan sulfate levels relative to untreated mice (black bar) in the mid-brain. There was no statistically significant difference in mid-brain heparan sulfate reduction between the treated mouse cohorts.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A fusion protein which comprises an N-terminal to C-terminal structure defined by formula I: AB-L1-RB-L2-C or formula II: RB-L1-AB-L2-C,
wherein for each of formula I and II:
AB comprises a domain that binds to human serum albumin (HSA);
L1, which may be present or absent, comprises a first linker amino acid sequence;
RB comprises a domain that binds to the extracellular domain of human IGF1R (hIGF1R);
L2, which may be present or absent, comprises a second linker amino acid sequence that is the same or different than the first linker amino acid sequence; and
C is a cargo moiety, optionally the cargo moiety comprises, consists essentially of, or consists of, the amino acid sequence of a polypeptide.

2. The fusion protein of claim 1, which is defined by formula I: AB-L1-RB-L2-C.

3. The fusion protein of claim 1, wherein each of AB and RB has a molecular weight of less than about any of 75 kDa, 50 kDa or 25 kDa, and optionally the cargo moiety has a molecular weight of about 1 kD to about 200 kD, or about 2 kD to about 100 kD.

4. The fusion protein of claim 1, wherein
(i) AB comprises a set of first, second and third amino acid sequences corresponding to the three complementarity determining regions CDR1, CDR2 and CDR3 of the heavy chain variable region of an anti-HSA antibody, optionally the set of first, second and third amino acid sequences are selected from:

| | (a) |
|---|---|
| (SEQ ID NO: 1) | |
| | GRTFIAYA, |
| (SEQ ID NO: 2) | |
| | ITNFAGGTT, |
| (SEQ ID NO: 3) | |
| | AADRSAQTMRQVRPVLPY; |
| | (b) |
| (SEQ ID NO: 41) | |
| | GFLLRSNTM, |
| (SEQ ID NO: 42) | |
| | IRPSGLT, |
| (SEQ ID NO: 43) | |
| | HTRPPFQRDS; |
| | (c) |
| (SEQ ID NO: 41) | |
| | GFLLRSNTM, |
| (SEQ ID NO: 42) | |
| | IRPSGLT, |
| (SEQ ID NO: 44) | |
| | ATRPPFQRDS; |
| | (d) |
| (SEQ ID NO: 45) | |
| | GRTFDNYVM, |
| (SEQ ID NO: 46) | |
| | ISGSGSIT, |
| (SEQ ID NO: 47) | |
| | AAGSRRTYYREPKFYPS; |
| | (e) |
| (SEQ ID NO: 48) | |
| | GSTFSSSSV, |
| (SEQ ID NO: 49) | |
| | ITSGGST, |
| (SEQ ID NO: 50) | |
| | NVAGRNWVPISRYSPGPY; |
| | and |
| | (f) |
| (SEQ ID NO: 48) | |
| | GSTFSSSSV, |
| (SEQ ID NO: 49) | |
| | ITSGGST, |
| (SEQ ID NO: 51) | |
| | AVAGRNWVPISRYSPGPY; |

or
(ii) AB comprises, consists essentially of or consists of an amino acid sequence selected from:
(a) QVQLVESGGGLVQAGGSLRLSCVASGRTFIAYAMGWFRQAPGKEREFVA AITNFAGGTTYYADSVKGRFTISRDNAKTTVYLQMNSLKPEDTALYYCA ADRSAQTMRQVRPVLPYWGQGTQVTVSS (SEQ ID NO:4) or a conservatively substituted variant thereof;
(b) QVQLVESGGGLVQPGGSLRLSCAASGRTFIAYAMGWFRQAPGKEREFVA AITNFAGGTTYYADSVKGRFTISRDNAKTTVYLQMNSLRAEDTAVYYCA ADRSAQTMRQVRPVLPYWGQGTLVTVSS (SEQ ID NO:5) or a conservatively substituted variant thereof;
(c) QVKLEESGGGLVQAGGSLRLSCVGPGFLLRSNTMGWYRQAPGKERELV AFIRPSGLTNYNDAVQGRFTISRDNAKNTVYLQMNALKPEDTAVYYCHT RPPFQRDSWGQGTQV TVSS (SEQ ID NO:52) or a conservatively substituted variant thereof;
(d) QVQLVESGGGLVQPGGSLRLSCAGPGFLLRSNTMGWYRQAPGKERELVA FIRPSGLTNYNDAVQGRFTISRDNAKNTVYLQMNSLRAEDTAVYYCHTR PPFQRDSWGQGTLV TVSS (SEQ ID NO:53) or a conservatively substituted variant thereof;
(e) QVQLVESGGGFVQAGGSLRLSCAASGRTFDNYVMAWFRQAPGKEREFV ASISGSGSITNYANSVKDRFTISRDSAKNAIYLQMNSLKPEDTALYYCAAG SRRTYYREPKFYPSWGQGTQVTVSS (SEQ ID NO:54) or a conservatively substituted variant thereof;
(f) EVQLVESGGGLVQPGGSLRLSCAASGRTFDNYVMAWFRQAPGKGLEFVS SISGSGSITNYANSVKDRFTISRDNSKNTLYLQMNSLRAEDTAVYYCAAG SRRTYYREPKFYPSWGQGTLVTVSS (SEQ ID NO:55) or a conservatively substituted variant thereof;
(g) QVKLEESGGGLVQAGGSLKLSCAASGSTFSSSSVGWYRQAPGQQRELVA AITSGGSTNTADSVKGRFTMSRDNAKNTVYLQMRDLKPEDTAVYYCNV AGRNWVPISRYSPGPYWGQGTQVTVSS (SEQ ID NO:56) or a conservatively substituted variant thereof; and
(h) EVQLVESGGGLVQPGGSLRLSCAASGSTFSSSSVGWYRQAPGKGLELVSA ITSGGSTNTADSVKGRFTISRDNSKNTLYLQMNSLRAEDTAVYYCNVAGR NWVPISRYSPGPYWGQGTLVTVSS (SEQ ID NO:57) or a conservatively substituted variant thereof.

5. The fusion protein of claim 4, wherein the first, second and third amino acid sequences in AB are: GRTFIAYA (SEQ ID NO:1), or a conservatively substituted variant thereof; ITNFAGGTT (SEQ ID NO:2), or a conservatively substituted variant thereof; and AADRSAQTMRQVRPVLPY (SEQ ID NO:3), or a conservatively substituted variant thereof.

6. The fusion protein of claim 1, wherein AB comprises, consists essentially of, or consists of the amino acid sequence from a sdAb.

7. The fusion protein of claim 6, wherein AB consists essentially of, or consists of an amino acid sequence selected from:
(i) SEQ ID NO:4;
(ii) SEQ ID NO:5; or
(iii) an amino acid sequence of the heavy chain variable region of an antibody that cross-competes with a sdAb consisting of SEQ ID NO:4 or SEQ ID NO:5 for binding to HSA.

8. The fusion protein of claim 1, wherein the fusion protein binds via AB to domain 1 (DI) or domain 2 (DII) of HSA and does not substantially inhibit binding of human FcRn to HSA.

9. The fusion protein of claim 1, wherein the fusion protein binds via the AB domain to HSA with a dissociation constant (K_{D}) affinity of less than about 0.1 nM to about 1,000 nM within a pH range of about 5.0 to about 7.4 as determined by surface plasmon resonance at 25° C., optionally wherein the K_{D} is about 0.5 nM to about 500 nM, about 1 nM to about 250 nM, about 5 nM to about 50 nM, about 10 nM to about 25 nM, or about 0.5 nM to about 1 nM.

10. The fusion protein of claim 1, wherein the fusion protein binds via AB to at least one mammalian serum albumin ortholog at 25° C. within a pH range of about 5.5 to about 7.4, optionally wherein the fusion protein binds via AB to two or more mammalian serum albumins selected from the group consisting of mouse serum albumin, rat serum albumin, hamster serum albumin, rabbit serum albumin, guinea pig albumin, pig albumin, cat albumin, dog albumin, and a non-human primate serum albumin, optionally wherein the non-human primate serum albumin is cynomolgus serum albumin or rhesus monkey serum albumin.

11. The fusion protein of claim 1, wherein the fusion protein binds via the RB domain to hIGF1R expressed on the surface of human brain endothelial cells, optionally wherein the fusion protein binds via RB to an epitope in the hIGF1R extracellular domain which comprises FENFLHNSIFVPR (SEQ ID NO:6).

12. The fusion protein of claim 1, wherein the fusion protein does not substantially bind to the human insulin receptor (h-IR), optionally wherein the fusion protein does not substantially inhibit binding of insulin, insulin growth factor 1 (IGF1) or insulin growth factor 2 (IGF2) to hIGF1R.

13. The fusion protein of claim 1, wherein the fusion protein binds via RB to hIGF1R with a K_{D} affinity of about 0.1 nM to about 1,000 nM within a pH range of about 5.0 to about 7.4, as determined by surface plasmon resonance at 25° C., optionally wherein the K_{D} is (i) about 0.2 nM to about any one of 500 nM, 250 nM, 100 nM, 50 nM, 25 nM or 10 nM; (ii) about 0.5 nM to about any one of 250 nM, 100 nM, 50 nM, 25 nM, 10 nM or 5 nM; or (iii) about 1 nM to about any one of 100 nM, 50 nM, 25 nM, 10 nM or 5 nM.

14. The fusion protein of claim 13, wherein the fusion protein binds via RB to hIGF1R with a K_{D} of 1 nM to 10 nM.

15. The fusion protein of claim 1, wherein the fusion protein binds via RB to at least one mammalian IGF1R ortholog at 25° C. and within a pH range of about 5.0 to about 7.4, optionally wherein the fusion protein binds via RB to two or more mammalian IGF1R proteins selected from the group consisting of mouse IGF1R, rat IGF1R, hamster IGF1R, rabbit IGF1R, guinea pig IGF1R, dog IGF1R, cat IGF1R and a non-human primate IGF1R, optionally wherein the non-human primate IGF1R is cynomolgus IGF1R or rhesus monkey IGF1R.

16. The fusion protein of claim 1, wherein
(i) RB comprises first, second and third amino acid sequences corresponding to the three complementarity determining regions CDR1, CDR2 and CDR3 of the heavy chain variable region of an anti-hIGF1R antibody, optionally the first, second and third amino acid sequences are selected from:

| | (a) |
|---|---|
| (SEQ ID NO: 7) | |
| | GRTIDNYA, |
| (SEQ ID NO: 8) | |
| | IDWGDGGX, where X is A or T, |
| (SEQ ID NO: 9) | |
| | AMARQSRVNLDVARYDY; |
| | (b) |
| (SEQ ID NO: 7) | |
| | GRTIDNYA, |
| (SEQ ID NO: 10) | |
| | IDWGDGGA, |
| (SEQ ID NO: 9) | |
| | AMARQSRVNLDVARYDY; |
| | (c) |
| (SEQ ID NO: 58) | |
| | EYPSNFYA, |
| (SEQ ID NO: 59) | |
| | VSRDGLTT, |
| (SEQ ID NO: 60) | |
| | AIVITGVWNKVDVNSRSYHY; |
| | and |
| | (d) |
| (SEQ ID NO: 61) | |
| | GGTVSPTA, |
| (SEQ ID NO: 62) | |
| | ITWSRGTT, |
| (SEQ ID NO: 63) | |
| | AASTFLRILPEESAYTY; |

(ii) RB comprises, consists essentially of or consists of an amino acid sequence selected from:
(a) QVKLEESGGGLVQAGGSLRLSCAASGRTIDNYAMAWSRQAPGKDREFV ATIDWGDGGARYANSVKGRFTISRDNAKGTMYLQMNNLEPEDTAVYSC AMARQSRVNLDVARYDYWGQGTQVTVSS (SEQ ID NO: 11) or a conservatively substituted variant thereof;
(b) QVQLVESGGGLVQPGGSLRLSCAASGRTIDNYAMAWVRQAPGKGLEWV ATIDWGDGGTRYANSVKGRFTISRDNSKNTMYLQMNSLRAEDTAVYYC AMARQSRVNLDVARYDYWGQGTLVTVSS (SEQ ID NO:12) or a conservatively substituted variant thereof;
(c) QVKLEESGGGLVQAGGSLRLSCVASEYPSNFYAMSWFRQAPGKEREFVA GVSRDGLTTLYADSVKGRFTMSRDNAKNTVDLQMNSVKAEDTAVYYCA IVITGVWNKVDVNSRSYHYWGQGTQVTVSS (SEQ ID NO:67) or a conservatively substituted variant thereof;
(d) EVQLVESGGGLVQPGGSLRLSCAASEYPSNFYAMSWFRQAPGKEREFVS GVSRDGLTTLYADSVKGRFTISRDNSKNTLYLQMNSLRAEDTAVYYCAI VITGVWNKVDVNSRSYHYWGQGTLVTVSS (SEQ ID NO:68); or a conservatively substituted variant thereof;
(e) QVKLEESGGGLVQAGGSLRLSCEVSGGTVSPTAMGWFRQAPGKEREFVG HITWSRGTTRVASSVKDRFTISRDSAKNTVYLQMNSLKSEDTAVYYCAAS TFLRILPEESAYTYWGQGTQVTVSS (SEQ ID NO:69) or a conservatively substituted variant thereof; and
(f) QVQLVESGGGLVQPGGSLRLSCAVSGGTVSPTAMGWVRQAPGKGLEWV GHITWSRGTTRYASSVKGRFTISRDNSKNTVYLQMNSLRAEDTAVYYCA ASTFLRILPEESAYTYWGQGTLVTVSS (SEQ ID NO:70) or a conservatively substituted variant thereof.

17. The fusion protein of claim 16, wherein the first, second and third amino acid sequences in RB are: GRTIDNYA (SEQ ID NO:7), or a conservatively substituted variant thereof; IDWGDGGA (SEQ ID NO:10), or a conservatively substituted variant thereof; and AMARQSRVNLDVARYDY (SEQ ID NO:9), or a conservatively substituted variant thereof.

18. The fusion protein of claim 17, wherein RB consists essentially of, or consists of:
(i) SEQ ID NO:11;
(ii) SEQ ID NO:12; or
(iii) an amino acid sequence of the heavy chain variable region of an antibody that cross-competes with a sdAb consisting of SEQ ID NO:11 or SEQ ID NO:12 for binding to hIGF1R.

19. The fusion protein of claim 1, wherein L1 and L2 are present, optionally wherein each of L1 and L2 is a linker peptide that is less than 50 amino acids in length, or is between about 15 and 30 amino acids in length, or between about 20 and 25 amino acids in length.

20. The fusion protein of claim 19, wherein one or both of L1 and L2 consist essentially of, or consist of, (GGGGS)m, wherein m is 4 or 5.

21. The fusion protein of claim 20, wherein each of L1 and L2 consists essentially of, or consists of: GGGGSGGGGSGGGGSGGGGS (SEQ ID NO:14).

22. The fusion protein of claim 1, wherein the cargo moiety is a cargo polypeptide selected from the group consisting of: an enzyme, a growth factor, a cytokine, or an antibody or antigen binding fragment thereof, optionally wherein the cargo polypeptide comprises the mature amino acid sequence encoded by a wild-type human gene.

23. The fusion protein of claim 22, wherein the cargo polypeptide is an acid alpha-glucosidase protein (GAA), an alpha-galactosidase A (GLA) protein, an alpha-L-iduronidase (IDUA) protein, an alpha-N-acetyl-glucosaminidase (NAGLU) protein, a beta-glucoronidase (GUSB) protein, a beta-glucosidase (GBA) protein, an iduronate-2-sulfatase (IDS) protein, an heparan-alpha-glucosaminide N-acetyltransferase (HGSNAT) protein, an N-acetylgalactosamine-6-sulfatase (GNS) protein, or an N-sulfoglucosamine sulfohydrolase (SGSH) protein, optionally wherein the cargo polypeptide is not an IDS protein.

24. The fusion protein of claim 22 or 23, wherein the cargo polypeptide is an IDUA protein, optionally wherein the IDUA protein consists essentially or consists of:
(i) amino acid 26, 27 or 28 to amino acid 653 of SEQ ID NO:22; or
(ii) amino acids 27-653 of SEQ ID NO:22.

25. The fusion protein of claim 24, which has an IDUA enzymatic activity that is within 80-120% of the corresponding enzymatic activity of wild-type human IDUA protein.

26. The fusion protein of claim 22 or 23, wherein the cargo polypeptide is an IDS protein, optionally where the fusion protein comprises, consists essentially of, or consists of SEQ ID NO:93 or SEQ ID NO:94.

27. The fusion protein of claim 26, which has an IDS enzymatic activity that is within 80-120% of the corresponding enzymatic activity of wild-type human IDS protein.

28. The fusion protein of claim 1, which comprises, consists essentially of, or consists of the amino acid sequence of an hIDUA fusion protein shown in FIG. 3A, FIG. 3B, FIG. 3C, FIG. 3D, FIG. 3E or FIG. 3F, optionally wherein the fusion protein consists essentially of, or consists of, SEQ ID NO:29, wherein each of m and n=4.

29. A polynucleotide which comprises a first nucleotide sequence that encodes the fusion protein of claim 1, optionally wherein the polynucleotide is one strand in an isolated double-stranded DNA molecule.

30. The polynucleotide of claim 29, wherein the first nucleotide sequence is operably linked to a nucleotide sequence encoding a secretory signal sequence for the fusion protein, optionally wherein the secretory signal sequence comprises, consists essentially of, or consists of, SEQ ID NO:79, SEQ ID NO:80, SEQ ID NO:81, SEQ ID NO:82, SEQ ID NO:83, SEQ ID NO:84, SEQ ID NO:85 or SEQ ID NO:86.

31. The polynucleotide of claim 30, wherein the secretory signal sequence consists essentially of, or consists of, SEQ ID NO:79.

32. The polynucleotide of claim 29, wherein the first nucleotide sequence is operably linked to a promoter sequence and a polyA signal sequence, optionally wherein the promoter sequence comprises, consists essentially of, or consists of, SEQ ID NO:32, SEQ ID NO:33 or SEQ ID NO:34 and optionally wherein the polyA signal sequence comprises, consists essentially of, or consists of, SEQ ID NO:35, SEQ ID NO:36 or SEQ ID NO:37.

33. The polynucleotide of claim 32, which comprises SEQ ID NO:39.

34. A genetically modified mammalian cell which is transiently or stably transfected with the polynucleotide of claim 29, optionally wherein the cell is derived from a human cell.

35. The genetically modified mammalian cell of claim 34, wherein the polynucleotide is inserted into at least one location in the genome of the mammalian cell.

36. The genetically modified mammalian cell of claim 34, which is derived from an RPE cell, optionally an ARPE-19 cell.

37. The genetically modified mammalian cell of claim 34, which is derived from an induced pluripotent stem cell (iPSC) or a mesenchymal stem cell (MSC).

38. A composition comprising a plurality of genetically modified cells, wherein each cell in the plurality is a genetically modified cell as defined by claim 34, optionally wherein the plurality of genetically modified cells is obtained from a culture of a monoclonal cell line.

39. An implantable device comprising at least one cell-containing compartment which comprises the genetically modified cell of claim 34 or the composition of claim 38 and further comprises at least one means for mitigating the foreign body response (FBR) when the device is implanted into the subject, optionally wherein the device comprises a spherical, two-compartment hydrogel capsule of about 0.75 mm to about 2 mm in diameter.

40. The implantable device of claim 39, wherein the cell-containing compartment comprises a polymer composition, wherein the polymer composition comprises an alginate covalently modified with a peptide, wherein the peptide consists essentially of or consists of GRGDSP (SEQ ID NO:89), GGRGDSP (SEQ ID NO:90) or GGGRGDSP (SEQ ID NO:91).

41. The implantable device of claim 39, wherein the cell-containing compartment is surrounded by a barrier compartment comprising an alginate hydrogel and optionally a compound of Formula (III) or a pharmaceutically acceptable salt thereof disposed on the outer surface of the barrier compartment,
wherein Formula (III) is 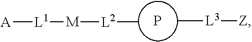
wherein
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{c})C(O)(C₁-C₆-alkylene)-, —N(R^{c})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R_{C})(R_{D}), —N(R^{C})C(O)R^{A}, —N(R^{C})(R^{D}), cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, C(O)N(R^{C1}), SR^{E1}, S(O)ₓR^{E1}, OS(O)ₓR^{E1}, —N(R^{C1})S(O)ₓR^{E1}, —S(O)_{X}N(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

42. The implantable device of claim 41, wherein the barrier compartment comprises an alginate chemically modified with 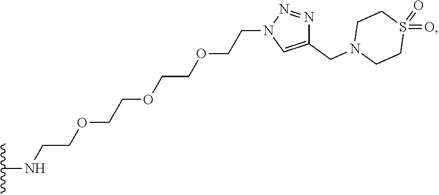 [SMILES: *NCCOCCOCCOCCn1cc(CN2CCS(=O)(=O)CC2)nn1]
or a pharmaceutically acceptable salt thereof.

43. A preparation of devices, wherein each device in the preparation is a device of claim 39.

44. A hydrogel capsule comprising:
(i) an inner compartment which comprises a plurality of the genetically modified cell of claim 34 encapsulated in a first polymer composition, wherein the first polymer composition comprises a hydrogel-forming polymer, optionally wherein the concentration of the genetically modified cell in the inner compartment is at least 40 million cells per ml of the first polymer composition; and
(ii) a barrier compartment surrounding the inner compartment and comprising a second polymer composition, wherein the second polymer composition comprises an alginate covalently modified with at least one compound of Formula (III) or a pharmaceutically acceptable salt thereof, optionally wherein the compound of Formula (III) is selected from a compound provided in the table below:

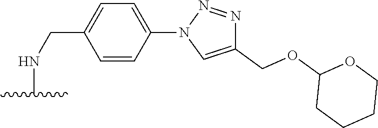 [SMILES: *NCc1ccc(-n2cc(COC3CCCCO3)nn2)cc1]

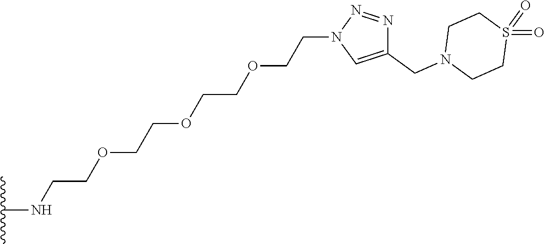 [SMILES: *NCCOCCOCCOCCn1cc(CN2CCS(=O)(=O)CC2)nn1]

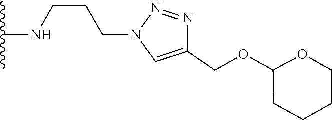 [SMILES: *NCCCn1cc(COC2CCCCO2)nn1]

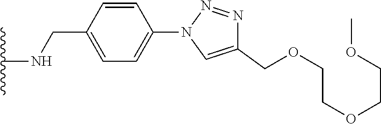 [SMILES: *NCc1ccc(-n2cc(COCCOCCOC)nn2)cc1]

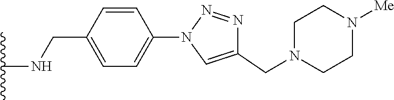 [SMILES: *NCc1ccc(-n2cc(CN3CCN(C)CC3)nn2)cc1]

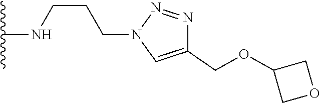 [SMILES: *NCCCn1cc(COC2COC2)nn1]

45. A capsule composition comprising a plurality of the hydrogel capsule of claim 44 in a pharmaceutically acceptable carrier.

46. The capsule composition of claim 45, wherein the genetically modified cells in the plurality of the hydrogel capsule express and secrete an IDUA fusion protein which consists essentially of, or consists of, SEQ ID NO:29, wherein each of m and n=4.

47. A pharmaceutical composition comprising the fusion protein of claim 1 and a pharmaceutically acceptable carrier.

48. The pharmaceutical composition of claim 47, wherein the fusion protein consists essentially of, or consists of, SEQ ID NO:29, wherein each of m and n=4.

49. A method of preventing or treating a disease or condition in the central nervous system (CNS) of a subject, which comprises:
(i) administering to the subject the pharmaceutical composition of claim 47;
(ii) implanting the device or device preparation of any one of claim 39; or
(iii)implanting the capsule composition of claim 45.

50. A method of treating a human subject diagnosed with Mucopolysaccharidosis type 1 (MPS-1) disease, comprising:
(i) providing the capsule composition of claim 46; and
(ii) disposing the capsule composition in the body of the subject, optionally wherein the disposing step comprises placing the capsule composition into the intraperitoneal space of the subject or into the greater sac of the peritoneal cavity.
